SEQUENCE LISTING (168 sequences)

SEQ ID NO: 1 - DNA, 2760 bp, Methylophilus methylotrophus
gtggctttaagctggaacgagataagaagaaaagctattgagttttctaaaagatgggaagacgcctcagatgaaaacagtcaagccaaaccctttttaatagattttttcgaagtttttggaataactaataagagagttgcaacatttgagcatgctgtgaaaaagttcgccaaggcccataaggaacaatctcgaggattcgtagatttgttttggcctggcattcttcttattgaaatgaaaagcagaggtaaagacctcgacaaagcgtatgaccaggcacttgattacttttctggcattgcagaaagagacttacccagatacgttttagtttgcgacttccagcgtttcagattaacagacctaataacaaaagagtcagttgaatttcttttaaaggacttataccaaaatgtgaggtcttttggttttatagctggttatcaaactcaagtaatcaagccacaagaccctattaatattaaggcggctgaacggatgggtaagcttcatgacaccctgaagttggttggatatgagggacacgctttagaactttatctagtgcgtttacttttttgcttattcgcagaagacacaactatttttgagaaaagtttattccaagaatatatcgagacaaagacgctagaggacggcagtgaccttgcacatcatatcaatacacttttttatgttctcaataccccagaacaaaaaagattaaagaatctagacgaacaccttgctgcatttccatatatcaatggaaaacttttcgaggagccacttccgccagctcagtttgataaagcaatgagagaggcattgcttgacttgtgctcattagattggagcaggatttcaccagcaatatttggaagtttattccaaagcattatggatgctaaaaagagaagaaatcttggggcacactacaccagcgaagcaaatattctcaagttaatcaagccattgtttcttgacgagctctgggtagagttcgagaaagttaaaaataataaaaataaattactagcgttccacaaaaaactaagaggacttacatttttcgaccctgcatgcggttgcggaaattttcttgtaatcacataccgagaactaagacttttagaaattgaagtgttaagaggattgcatagaggtggtcaacaagttttggatattgagcatcttattcagattaacgtagaccagttttttggtatcgaaatagaggagtttcccgcacagattgctcaggttgctctctggcttacagaccaccaaatgaatatgaaaatttcagatgagtttggaaactactttgcccgtatcccactaaaatctactcctcacattttgaatgctaatgctttacagattgattggaacgatgttttagaggctaaaaaatgttgcttcatattaggaaatcctccatttgttggtaaaagtaaacaaacaccgggacaaaaagcggatttactatctgtttttggaaatcttaaatccgcttcagacttagacctagttgctgcttggtatcccaaagcagcacattacattcaaacaaatgcaaacatacgctgtgcatttgtctcaacgaatagtattactcaaggtgagcaagtatcgttgctttggccgcttctgctctcattaggcataaaaataaactttgctcacagaactttcagctggacaaatgaggcgtcaggagtagcggcggttcactgcgtaattatcggatttgggttgaaggattcagatgaaaaaataatctatgagtatgaaagtattaatggagaaccattagctattaaggcaaaaaatattaatccatatttgagagacggggtggatgtgattgcctgcaagcgtcagcagccaatctcaaaattaccaagcatgcgttatggcaacaaaccaacagatgatggaaatttcctatttactgacgaagaaaaaaaccaatttattacaaatgagccatcttccgaaaaatacttcagacggtttgtgggcggggatgagttcataaacaatacaagtcgatggtgtttatggcttgacggtgctgacatttcagaaatacgagcgatgcctttggtcttggctaggataaaaaaagtccaagaattcagattaaaaagctcggccaaaccaactcgacaaagtgcttcgacaccaatgaagttcttttatatatctcagccggatacggactatctgttgatacctgaaacatcatctgaaaacagacaatttattccaattggttttgttgatagaaatgtcatttcaagtaacgcaacgtatcatattcctagtgctgaacctttgatatttggcctgctttcatcgaccatgcacaactgctggatgagaaatgtaggaggaaggttagaaagtcgttatagatattctgccagcctggtttacaacacgtttccatggattcaacccaacgaaaaacaatcgaaagcgatagaagaagctgcatttgcgattttaaaagctagaagcaattatccaaacgaaagtttagctggtttatacgacccaaaaacaatgcctagtgagcttcttaaagcacatcaaaaacttgataaggctgtggattctgtctatggatttaaaggaccaaacacagaaattgctcgaatagcttttttgtttgaaacataccaaaagatgacttcactcttaccaccagaaaaagaaattaagaaatctaagggcaaaaattaa

SEQ ID NO: 2 - protein, 919 aa, Methylophilus methylotrophus
MALSWNEIRRKAIEFSKRWEDASDENSQAKPFLIDFFEVFGITNKRVATFEHAVKKFAKAHKEQSRGFVDLFWPGILLIEMKSRGKDLDKAYDQALDYFSGIAERDLPRYVLVCDFQRFRLTDLITKESVEFLLKDLYQNVRSFGFIAGYQTQVIKPQDPINIKAAERMGKLHDTLKLVGYEGHALELYLVRLLFCLFAEDTTIFEKSLFQEYIETKTLEDGSDLAHHINTLFYVLNTPEQKRLKNLDEHLAAFPYINGKLFEEPLPPAQFDKAMREALLDLCSLDWSRISPAIFGSLFQSIMDAKKRRNLGAHYTSEANILKLIKPLFLDELWVEFEKVKNNKNKLLAFHKKLRGLTFFDPACGCGNFLVITYRELRLLEIEVLRGLHRGGQQVLDIEHLIQINVDQFFGIEIEEFPAQIAQVALWLTDHQMNMKISDEFGNYFARIPLKSTPHILNANALQIDWNDVLEAKKCCFILGNPPFVGKSKQTPGQKADLLSVFGNLKSASDLDLVAAWYPKAAHYIQTNANIRCAFVSTNSITQGEQVSLLWPLLLSLGIKINFAHRTFSWTNEASGVAAVHCVIIGFGLKDSDEKIIYEYESINGEPLAIKAKNINPYLRDGVDVIACKRQQPISKLPSMRYGNKPTDDGNFLFTDEEKNQFITNEPSSEKYFRRFVGGDEFINNTSRWCLWLDGADISEIRAMPLVLARIKKVQEFRLKSSAKPTRQSASTPMKFFYISQPDTDYLLIPETSSENRQFIPIGFVDRNVISSNATYHIPSAEPLIFGLLSSTMHNCWMRNVGGRLESRYRYSASLVYNTFPWIQPNEKQSKAIEEAAFAILKARSNYPNESLAGLYDPKTMPSELLKAHQKLDKAVDSVYGFKGPNTEIARIAFLFETYQKMTSLLPPEKEIKKSKGKN

SEQ ID NO: 3 - DNA, 2802 bp, unknown
  Environmental sample Sargasso Sea
atggctgccctctcgttcccggaaatccgcacccgcttgcaagcgttcgccaaacaatggaagcaagcggagcgcgaaaacgccgacgcaaagttgttttgggcacggttttacgagtgcttcggcatccgcccggagtccgcgaccatctacgagaaggcggtggacaaacttgatggctcgcggggcttcatcgactcgtttattccggggctgttgatcgtcgagcacaagagtaagggcaaggacctgaactcggccttcacccaagcctccgactacttcacggcgctggctgaaggtgagcgtccgcggtacatcatcgtgtcggatttcgcccgttttaggctgtacgacctgaaaaccgacacccaggtggagtgcaaactcgcggacatctccaagcacgccggctggttccggttcctagtcgagggtgaggctacgccagaaatcgtcgaggagtcaccgatcaaccggcaggctgcgtacgccgtctcgaagttgcacgaggcgctgttgcaggcaaacttccgaggccgtgacttggaggtgttcctgacgcggctgctgttctgcttcttcgccgatgatactggcatctttggccaagacggtgtcttccgtcggtacgtcgaagccacgcgcgacaatggccgggacaccgggcaaagcctcgcgatcctgtttgacgtgctggacacgccggataaccagcgttcgtccaacctggacgagcacctgaccgcgttcgcctacatcaacgggtcgctgttttctgagcgtacgcgtatcccgtcattcgacgcggacatgcgaaccttgttggtgaagtgcgcagaactggactggagcgggatcagccccgcgatcttcggggcgatgtttcaaggcgtgctggaagcccacacgccagacgaaaagcgccaggccagtcgtcgggaactgggtgctcactacacctcggaacgtaacatcttgcgggtgatcaatccgctgttcatggacgacttgcgcgtagagttcgagagggcgcgcaggaacaagccccgattgcaggcgctgtacgagaagttgccaacgctcacattcttcgatcccgcgtgcggctgcgggaacttcttggtgatcgcgtaccgggaactgcgccgtctggaaaacgatgtcatcgccgcactgttcgcggacttccagcacggcaagggtttgctagacgtgtcgacgctctgcagggttcgggtcaatcagttttacggcctggagatcgacgacgcggcggcgcacatcgcgcgcgtggccatgtggatcacggaccatcagatgaacctggagtcggcagaccgcttcggcaatactcgcccgacagttccgctggtcgacactccccacattcacaaagagaacgcgctacgcgccgattggacatcggttctcgcgcccgcgcagtgttcgtacgtgatgggcaatcctccgttcgtaggtgcgaagtggctgaacgaggaacagcgtgccgacgcccgggcggtgttcgctaacgttaagaacggcggactgttggactacgtggccgcttggtatgttaaggcgctggcttacatccaagctaacccggccatcgacgtggcgtttgtttcaaccaactcgatcacgcaaggtgagcaagtgtcagccctctggccgacgctgctgcaaggtggggtaaaaatccgctttgcccaccggacgtttcagtggagcaacgaagggaaaggcaatgctgccgtccattgcgtcatcatcggcttcggcctgcgtgtcccggatcgctgcacgatcttcgattacagccacgacatcaaggccgacctgggttcggttcttcacgcgtctcgcatcaatccgtacttggtggacgccccggacgtcgtgctgacaaatcggcgtgcgccgatttgtcaggtgccggaaatcggcatagggaacaaacccatcgacggcgggcattacctgtttactgacgaaggaaaggccgcgttcctggccgtcgagccgaaagccgccccgtttttccatcgctgggtcggcgcggaagagttcatcaacaacacaagccgttggtgtctatggttgggtaacgcgaagccgcatgaactccgcgcgctccccgaatgtatgaagcgcgttgaggcagtgcgtcaatatcgcctcgccagccccagcgctccgacgcagaaactggccgagaccccgacccggtttcacgtcgagttcatgccagacgccccgttcatggtgatccctgaagtatcgtccgaacgtcgcgagttcatcccactggggtacctgcaaccgccaacgctggcgagcaacaaactgcgcttgatgccagatgcgacgctgtatcacttcgcggtgttgaactccaccatgcatatggcttggacacgggcggtatgcggccggctggaaagccgatatcagtactcggtcaccatcgtgtacaacaactttccatggcccagtccatccgacgcccaacttgaagcgctggaagcggcaggacaggcaatcctcgatgcccaggctatgtatttggaccagggttcatcgctagccgatctgtacgatccgcgcacgatgccgtcagaacttcgcaaggcccatgctgcgaacgatcgcgccgttgatgcggcgtacaagttcaagggcgacaagtccgacgccgtgcgggtcgctttcttgtttagcctgtacggaaggttgacgagccttcttccgtccgagaagccgaagcgtgctcggaaagagaaagcagtcgcgtaa

SEQ ID NO: 4 - protein, 933 aa, unknown
  Environmental sample Sargasso Sea
MAALSFPEIRTRLQAFAKQWKQAERENADAKLFWARFYECFGIRPESATIYEKAVDKLDGSRGFIDSFIPGLLIVEHKSKGKDLNSAFTQASDYFTALAEGERPRYIIVSDFARFRLYDLKTDTQVECKLADISKHAGWFRFLVEGEATPEIVEESPINRQAAYAVSKLHEALLQANFRGRDLEVFLTRLLFCFFADDTGIFGQDGVFRRYVEATRDNGRDTGQSLAILFDVLDTPDNQRSSNLDEHLTAFAYINGSLFSERTRIPSFDADMRTLLVKCAELDWSGISPAIFGAMFQGVLEAHTPDEKRQASRRELGAHYTSERNILRVINPLFMDDLRVEFERARRNKPRLQALYEKLPTLTFFDPACGCGNFLVIAYRELRRLENDVIAALFADFQHGKGLLDVSTLCRVRVNQFYGLEIDDAAAHIARVAMWITDHQMNLESADRFGNTRPTVPLVDTPHIHKENALRADWTSVLAPAQCSYVMGNPPFVGAKWLNEEQRADARAVFANVKNGGLLDYVAAWYVKALAYIQANPAIDVAFVSTNSITQGEQVSALWPTLLQGGVKIRFAHRTFQWSNEGKGNAAVHCVIIGFGLRVPDRCTIFDYSHDIKADLGSVLHASRINPYLVDAPDVVLTNRRAPICQVPEIGIGNKPIDGGHYLFTDEGKAAFLAVEPKAAPFFHRWVGAEEFINNTSRWCLWLGNAKPHELRALPECMKRVEAVRQYRLASPSAPTQKLAETPTRFHVEFMPDAPFMVIPEVSSERREFIPLGYLQPPTLASNKLRLMPDATLYHFAVLNSTMHMAWTRAVCGRLESRYQYSVTIVYNNFPWPSPSDAQLEALEAAGQAILDAQAMYLDQGSSLADLYDPRTMPSELRKAHAANDRAVDAAYKFKGDKSDAVRVAFLFSLYGRLTSLLPSEKPKRARKEKAVA

SEQ ID NO: 5 - DNA, 2727 bp, Sulfurimonas denitrificans
atgataagcttaagagagatacgagaacgaagcataaagtttgccaaagagtgggagggtgcttctcatgaaaaacaagaagcgcagagtttttggatagatttttttaaaatatttgatgtaagtccacgaagtatgcagtttgagtatcccatcaaaaaaatagacggctcttatggttacatagatgttttttggagagggcagcttcttatagagcaaaaaagcagaggcaaggatttagtaaaggcaaaagaacaagcgttagagtaccttccaaatctaaaacagagagatttaccgaagtttattttggtttgtgattttgtaagcttctatctttacgatttggacacaaatcaagattataaatttctactccatgagttaccaaaaaatatagagctgttttcatttatagcaggatacacaaaaaaaacctacaaagaagaggaaccgaccaaccgcaaagccgccgaacttatgggtaaacttcatgacaagctacttgaaaacggttacagcggacatcaactcgaactctttttaacaaggcttcttttttgtatgtttgcagaagatacgggcatatttgctaaaaactcttttcgtgaatttatagaaaatcaaacagatgagagcggcagagatttaggctcgcagataagctacctctttgagctttttgacactccaaatgaggagcgacaaaaaaatcttgatgagagttttactcagtttccttacatcaacggctcaatttttacagaacagctcaaaacagcccactttgaccgctccatgcgtgaaatgcttttggatgcgtgtgcctttgactggagtttgataagtccttccattttcggttcaatgtttcaagcttctatggacgttagtaaaagaggcgaactcggtgcgcactttacaagtgagacaaatatattaaaagccatcaaaccgctatttttggatgaacttagcgaagagtttgcaaaaataaaaaacaacccaaaacagcttcaaatttttcatgcaaaaatctcaaatctcaaatttttagacccagcatgtggaagtgggaactttttggtaatcgcttacagagagttgaagcttgtagagtttgaagtgctgaaatctcttaaaatactcacacaactcgtccatatagaccaattttatggtttcgagatagaagagttgccaagtcgaataactcaaactgcgatgcttctcatcgaccatcaaatgaacctgctttttgctcaaatgtttggagagccacattttaatatccccataaaagatagtgcaaatatttttaatgtcaatgctttgagggtggattgggaaaagattttggatggtgtgaaaattgattttattattggaaatccgccgtttttaggttcaaaaatgcaatctaaagagcaaaaagaggatatggcagaggtttttagcggtgttaaaaatggaaaagaacttgattttgtaacggcttggtatataaaatctgcaaaatatttacaaggtaaaaacacaaaagtagccttagtttcaacgaactccattacgcaaggcgaacaagtagggattttgtggcaagagatgtttaacaaatataaaatcaaaatccactttgcacacaaaacttttaaatggaataatgatgcaaaaggcgttgcacaagtttattgtgtaattatcggttttgcggggtttgacatcaaagaaaaaagactttttgagtatgagagcgtaaaatctgaaccgcatgagataaaagttgcaaatataaatccctatcttgtaaacggagatgatttttttatcagctcaagaagaaagcatatacagagctttatacctcaaatagtttttggaagtatgccaaatgacggtggtaacctgctttttgacgataaagaaaaagaggagtttttagcccttgaaccaaaagcagagctgtacatgaagcctcttatctctgcaaaagagtatcttaacggcaaaacaagatggtgtttatggctaaaagattgtccgccaaatgaactaaaatctatgcccaaagtgattgagagagttgaaaatatcagaaaacttaggaacgaaagctcaagagaagcaactcaaaaattagcaaagttcccagcactttttggagaagatagacagcctgagagtgattatatttttattcctcgtgtatcgtcagaaaacagagattatattccaatggaattttttacaaaagattttatttgtggagatactggacttgccgttccaaatgccacactttttcatttcggaattttgacttcaaaaatgcacatggactgggtgcggtatgttgctggaagattaaaaagtgattatagatattcaaatgaaattgtttataacaacttcccttttcctttagaaataaacgacaaacaaaaagatcaaatcgaacaattagcacaaaatattctagacataagagccgaatttgtaggaagctctttagccgatttgtacaatcctctaactatgccaccaaaactcctaaaagctcacgaaacgctagacagagcagtagataaactctactcaaaaacactcttcaaaacagatacagaaagagtcgcccatttgtttgaattaaataaacaacttactagcttgattgtggaaaatgagaaaaaagctaaaaaagttaaaaaaataataacaaaatga

SEQ ID NO: 6 - protein, 908 aa, Sulfurimonas denitrificans
MISLREIRERSIKFAKEWEGASHEKQEAQSFWIDFFKIFDVSPRSMQFEYPIKKIDGSYGYIDVFWRGQLLIEQKSRGKDLVKAKEQALEYLPNLKQRDLPKFILVCDFVSFYLYDLDTNQDYKFLLHELPKNIELFSFIAGYTKKTYKEEEPTNRKAAELMGKLHDKLLENGYSGHQLELFLTRLLFCMFAEDTGIFAKNSFREFIENQTDESGRDLGSQISYLFELFDTPNEERQKNLDESFTQFPYINGSIFTEQLKTAHFDRSMREMLLDACAFDWSLISPSIFGSMFQASMDVSKRGELGAHFTSETNILKAIKPLFLDELSEEFAKIKNNPKQLQIFHAKISNLKFLDPACGSGNFLVIAYRELKLVEFEVLKSLKILTQLVHIDQFYGFEIEELPSRITQTAMLLIDHQMNLLFAQMFGEPHFNIPIKDSANIFNVNALRVDWEKILDGVKIDFIIGNPPFLGSKMQSKEQKEDMAEVFSGVKNGKELDFVTAWYIKSAKYLQGKNTKVALVSTNSITQGEQVGILWQEMFNKYKIKIHFAHKTFKWNNDAKGVAQVYCVIIGFAGFDIKEKRLFEYESVKSEPHEIKVANINPYLVNGDDFFISSRRKHIQSFIPQIVFGSMPNDGGNLLFDDKEKEEFLALEPKAELYMKPLISAKEYLNGKTRWCLWLKDCPPNELKSMPKVIERVENIRKLRNESSREATQKLAKFPALFGEDRQPESDYIFIPRVSSENRDYIPMEFFTKDFICGDTGLAVPNATLFHFGILTSKMHMDWVRYVAGRLKSDYRYSNEIVYNNFPFPLEINDKQKDQIEQLAQNILDIRAEFVGSSLADLYNPLTMPPKLLKAHETLDRAVDKLYSKTLFKTDTERVAHLFELNKQLTSLIVENEKKAKKVKKIITK

SEQ ID NO: 7 - DNA, 2865 bp, Neisseria lactamica ST640
atgccgtctgaaagcacacttcagacggcattttcccaacaggcacgcatcatgaccccagacctccaaaccctccaacacaacgccgaacaattcatccgcgactgcgaacccctgcattacgaaatgggtcatgcccaaaaattcatcgccgccctatgcaaagtgtacggcctcgatgcccacttcgccgtccaatacgaacaccgcgtccgcaaagctgacctcaaaggcatcaaccgcatcgacggcttcttccccggcctgctgatgatagaaatgaaatccgccggcgaagacctcgaagccgccttcatccaagccctggaatacgtccaactcatagagcgcatcgaagacaagccccgccacatcctcgtctccgacttcaaaaacctccacctttacgagctgaatcaaggatttaccggcatcgtcctcgacaaaaccctcaaaatcaaactcaccggcttccgcgcccacgtccaagacttcgccttcatcgcaggctacgaagccgccattgccgagcgcaacgaagccctgaccatagccgccgccgccaaactcgccgccctgcaccaagaattccacaaacaaggctaccaaggcgcagaactccaaaccatgctcgtccgcatcctcttctgcctctttgccgacgacaccggactcttcgcccaaaacaaagccttcgagcagcttgtcgaagaaagcctcgccgacggcgcagacctcggcagccgcctcaacgccctctacaaatggcttgacacccccgaagacaaacgccgcaccaccccgcgggccctgcttgaccaatacagcggcttccgcctcaaattcccctacatcaacggcaaactcttttcagacggcatagacgaattcgtcttcaacgcctccatgcgccgcaccctcctcgaatgctgcgaaatcgactggagcctcatctcccccgacatcttcggcacactcttccaaaacatcatggaaaacgccgacgcactcggcggcggcaaaaaatctgcccaccgccgcgaactcggcgcacactacaccagcgaaaaaaacatcaaacgcgccatcgcccccctctttctcgaccgcctcaaagccgagcttgagcaggctgccggcgaccccaaaaaactcgcccgctacattacccgcctgcaaaccctccaaatcctcgatcccgcctgtggctgcggcaacttcctcatcgtcgcctaccgcgaaatccgcctgctcgaaatgcaggcaatccgccaactcgcccgcatccccggcgcgcagcaaatgcagtcccaatgcgacgtccaccaattccacggcatcgaaatcgaccccgccgccgtcgaaatcgccaccgttgccatgtggctcaccgaccaccagatgaaccgcctctaccaagacggctacaaacgcatccccctcgcccacaaagccgacatccgctgcgccaacgccctccaaaccgactgggcagacaccatatccccccaaaacctcgactatatcgtcggcaaccccccgtttttaggcaaaaaagaacaaaatgccgaacagaaaaaagatatggaaaaagtggtaggacatctcaaaggttcggggattctcgattacgttacggcttggtatttcaaagcaaacgaattgatgaaacacaaccccaaaatccgcaccgccttcgtttccaccaactccatcacccaaggcgaacaagtccccgccctctggaagcccctgctttcagacggcatccgcatccgcttcgcccaccgcaccttcaaatggaacaacgaaggcaaaggcaccgccgccgtccactgcgtcatcatcggcttcgaccgcgacgaaatccaaaaaggcgaacgcctcagcctttgggattacagccaaggcatcggcggcgacggcaaagaacaccaagtccgcaaaatcaatccttatctgcttgaagcagacaatatcctgcccgccaaaagaagccgccccgtatcagcagatgttccggcaatgaattacggaagtatgccgattgacaacggcttgctgattctgtcccaagaagcgtttcagacggcattaaacgaagaccccgaaaatagcgaactgatccgcccctatatgggcggcagcgaattcctgaacaatgaaaaacgttattgcctgtggttggaaaacgtcgatcaagaacgcctgtcccaaagcaaatttgcttcggaacgggtagggcaagtcagagcctaccgcctgtccagttcgcgcgcagccactgtaaaactggctggaacaccgcacttgttcggcgaaatccgccaacctgacagccgttatctgctgttgcccaaagtgtcgtctgaaaaccgccgttttcttcccatcggttacatcgaacctgaaaccattgccaacggaagcgcattgattatccccaacgccaccctctgccacttcggcatcctaagctccaccatgcacaacgccttcatgcgcaccgtcgcaggcagattggaaagccgttaccaatactcggcaagtatcgtgtacaacaatttccccttccccgaaaacccctgccgcaccgccatcgaaaccgcagcccaagccgtcctcgacgcacgcgccgccgaaaccgaacgcatccgccgcctcaaccggatcctgcccgaaaaagaacaccgccccatgcccacacccgccaccctctacaaccccgacaccatgccccccgccctcgccgccgcccacaacgccctcgacgatgccgtggacgaagcctacggctacacgggcggcaacagcgacagcgaacgcaccgccttcctcttccgcctctacaaaaatgccgtctga

SEQ ID NO: 8 - protein, 954 aa, Neisseria lactamica ST640
MPSESTLQTAFSQQARIMTPDLQTLQHNAEQFIRDCEPLHYEMGHAQKFIAALCKVYGLDAHFAVQYEHRVRKADLKGINRIDGFFPGLLMIEMKSAGEDLEAAFIQALEYVQLIERIEDKPRHILVSDFKNLHLYELNQGFTGIVLDKTLKIKLTGFRAHVQDFAFIAGYEAAIAERNEALTIAAAAKLAALHQEFHKQGYQGAELQTMLVRILFCLFADDTGLFAQNKAFEQLVEESLADGADLGSRLNALYKWLDTPEDKRRTTPRALLDQYSGFRLKFPYINGKLFSDGIDEFVFNASMRRTLLECCEIDWSLISPDIFGTLFQNIMENADALGGGKKSAHRRELGAHYTSEKNIKRAIAPLFLDRLKAELEQAAGDPKKLARYITRLQTLQILDPACGCGNFLIVAYREIRLLEMQAIRQLARIPGAQQMQSQCDVHQFHGIEIDPAAVEIATVAMWLTDHQMNRLYQDGYKRIPLAHKADIRCANALQTDWADTISPQNLDYIVGNPPFLGKKEQNAEQKKDMEKVVGHLKGSGILDYVTAWYFKANELMKHNPKIRTAFVSTNSITQGEQVPALWKPLLSDGIRIRFAHRTFKWNNEGKGTAAVHCVIIGFDRDEIQKGERLSLWDYSQGIGGDGKEHQVRKINPYLLEADNILPAKRSRPVSADVPAMNYGSMPIDNGLLILSQEAFQTALNEDPENSELIRPYMGGSEFLNNEKRYCLWLENVDQERLSQSKFASERVGQVRAYRLSSSRAATVKLAGTPHLFGEIRQPDSRYLLLPKVSSENRRFLPIGYIEPETIANGSALIIPNATLCHFGILSSTMHNAFMRTVAGRLESRYQYSASIVYNNFPFPENPCRTAIETAAQAVLDARAAETERIRRLNRILPEKEHRPMPTPATLYNPDTMPPALAAAHNALDDAVDEAYGYTGGNSDSERTAFLFRLYKNAV

SEQ ID NO: 9 - DNA, 2805 bp, Psychrobacter sp. PRwf-1
atgagtatagattacaagcacgtcagacaacaattacaacaaatcgttcacgactataaagactctgagggctatgagcgtggccaaagccaaaacttttggactcaagtgtttaatgcttatggcgtgtctggccaaactcaaactaaagcatttgaacatcgtcttaaagacaaatctaatcaaaaatacgttgatgctttcatccccaaattggtcataattgagcaaaaaagtcgtggtgtagatttaaataaagcctatacacaggtgtctgagtattacgatcgtattaacgctaaagacaagcctagatacatcatcttatgcaacttcgatgaaatttggctgtatgacatcaacaacccattagatattaaaaagcatcaatgtccactctctgatctgccaaacaacgctgaatggttcgagttcttatcgcctgaaagccaacaatctaatgagattatcgaagaaaaccccatcaaccgacaagctactgaaaagctagctaaactgcaccaggctttcattgaggatggtgtagatcctgatgaattagccttatttttaacacgcctaatcttctgtttctttgctgacgacaccgctatttttggtaaaaaacacgtactgcacaatttgttaaaaaaccatgcagccaccgatggtagtaacttacagcagatactaaccactttatttgacacattaaacactgagcatcgttcaagcagattgcctgagcattatgctcaattcgcctatatcaatggcggtctttttgaagaaactatcaacatcccttatttcgatgaaaagctatataacctagttatggagtgtgatgcactcgattggactgagattagccctgcaatcttcggttcgatgttccagagtgtattggatgctagtgggggagatagcactgaggataaacggcgtgagtttggtgctcactacaccagtgagaagaatattctaaaagtcatcaactcattgtttttacaagagttacgtgatgagttttctaagtgtactaacaacacaccaagagccgtacagctatatgaaaaactgcctacactaaagttctttgaccctgcttgtggttgcggtaactttttaatcattgcctatcgtgaattacgtctattagaaaaccagttgattgccaagatatttggtgatcaaaagggattacttgatattagcagtatgtgtaatgtgaccgtagatcagttttacggcattgagattgaacctcatgccgttcatatcgctcgtgttgctatgtggatcactgaccaccagttaaacatgaccactgcggagcgttttggcacaaccagaccgaccacaccgattgtttatagccctcatattattgaaggtaatgccttacaaatagattgggaaacagtcttacctgccaatgattgtagctatgtaatgggaaatcctccatttatcgggaaatccaatcaaagttctgaacaaaagtcagatataaaattagtagctagccatattaaaaatcacaagtctttagactatgtagcaggttggtatataaaatccatgcattatatgcaatcagttaataatgcaaatcattatatagatacagcttttgtatcaacaaactcgatagttcaaggtgagcaagttgacatcctatggagatatctaattgatgattgcaaaggccatataaacttcgcacatcatacctttaaatggagcaatgagggcaaagggatagctgcggttcattgcattattgttggcttttctttagtagaaaagaaagagaaaaccatcttcgaatactctgacatttcgtcagaaccaagccccaaaaaagctagaaccatcaatgcatatttaactgacgctccaatagttttctttagtagaagaagtaaacaagtttccaacgaaagtagtatggttagtggcaacaaggcaacagatggaggtaacttaattctgtcagactcagagtatatagatttaattaattcagagccattagctaagaaatacattaaacgttttatgatgggctatgaatttcttaacaatattaagcgatggtgtctgtggtttgataatgttgacccaatacaattaagtaaagatcttgaaaaaatgcctcttattaaaaagcgcattcataatgtcaaagaactgcgtttgaacagcactaaaaagtctactgtcaaaaaggcagaaacacctcatttgttcgatgaaagacggcatactaataaaccttacgttgcaatacccgtcgtatcatcagagaacagaagatttataccgattggctttattgatggtaacaccgtagcaggtaacaagttatttgtaattgtagatggtaatacctatcagttcggtactctgtctagcagtatgcataacgcatttatgagactaacagcgggtagaatgaaaagtgactatagctattcaagcaccattgtttataacaactttccttacccatttatggctgatgatcatagtgataaagcacaaaaagcgagagaaagcatagctaaggcttcacaacaggttttagatgctcgtaaacactatcaagacggtagtgagaacgcaccaaccctggctcagttatacaatacctatctaattgatccatatccactactaaccaaggctcataaagcgttagataaggccgttgatagtgcttatggttatcgtggcaaaggtgatgatgcgagtcgagtcgagtttttgattaagaagattgctgagttaaaaaattaa

SEQ ID NO: 10 - protein, 934 aa, Psychrobacter sp. PRwf-1
MSIDYKHVRQQLQQIVHDYKDSEGYERGQSQNFWTQVFNAYGVSGQTQTKAFEHRLKDKSNQKYVDAFIPKLVIIEQKSRGVDLNKAYTQVSEYYDRINAKDKPRYIILCNFDEIWLYDINNPLDIKKHQCPLSDLPNNAEWFEFLSPESQQSNEIIEENPINRQATEKLAKLHQAFIEDGVDPDELALFLTRLIFCFFADDTAIFGKKHVLHNLLKNHAATDGSNLQQILTTLFDTLNTEHRSSRLPEHYAQFAYINGGLFEETINIPYFDEKLYNLVMECDALDWTEISPAIFGSMFQSVLDASGGDSTEDKRREFGAHYTSEKNILKVINSLFLQELRDEFSKCTNNTPRAVQLYEKLPTLKFFDPACGCGNFLIIAYRELRLLENQLIAKIFGDQKGLLDISSMCNVTVDQFYGIEIEPHAVHIARVAMWITDHQLNMTTAERFGTTRPTTPIVYSPHIIEGNALQIDWETVLPANDCSYVMGNPPFIGKSNQSSEQKSDIKLVASHIKNHKSLDYVAGWYIKSMHYMQSVNNANHYIDTAFVSTNSIVQGEQVDILWRYLIDDCKGHINFAHHTFKWSNEGKGIAAVHCIIVGFSLVEKKEKTIFEYSDISSEPSPKKARTINAYLTDAPIVFFSRRSKQVSNESSMVSGNKATDGGNLILSDSEYIDLINSEPLAKKYIKRFMMGYEFLNNIKRWCLWFDNVDPIQLSKDLEKMPLIKKRIHNVKELRLNSTKKSTVKKAETPHLFDERRHTNKPYVAIPVVSSENRRFIPIGFIDGNTVAGNKLFVIVDGNTYQFGTLSSSMHNAFMRLTAGRMKSDYSYSSTIVYNNFPYPFMADDHSDKAQKARESIAKASQQVLDARKHYQDGSENAPTLAQLYNTYLIDPYPLLTKAHKALDKAVDSAYGYRGKGDDASRVEFLIKKIAELKN

SEQ ID NO: 11 - DNA, 2859 bp, Corynebacterium striatum M82B
atggttatggcccctacgactgtttttgaccgcgctaccattcgccacaatctcaccgaattcaaactccggtggcttgaccgcattaagcaatgggaggcggaaaaccgacccgcaaccgagtcgagtcacgaccaacagttctggggtgacctgctcgactgcttcggtgtcaacgcccgcgacctgtacttgtaccaacgcagcgctaaacgcgcttcgacggggcgcaccggcaagatcgacatgtttatgccgggcaaagtcataggcgaggctaagtccctcggcgtcccgctcgatgatgcttatgcccaagctttggattatttgctgggcggtactatcgcgaactcgcacatgccggcctatgttgtctgctccaacttcgagaccctgcgggttacccgtcttaaccgcacctatgtcggcgatagcgccgactgggacattacattccctttagctgagattgacgagcacatcgaacaactcgcttttctcgccgactatgaaacctccgcctaccgggaggaagaaaaggcttccctggaagcctctcggttaatggtggagctcttccgcgccatgaacggcgacgacgtggacgaggcagtaggcgatgacgctcccaccacgccggaggaagaagacgagcgcgtcatgcgcacctctatctacctcacccgaatcctcttccttctcttcggcgacgacgcaggactctgggataccccgcatttgtttgcggactttgtgcgcaatgaaaccaccccagaatcgctcggcccgcagctcaatgagctatttagcgtgcttaataccgccccggaaaagcggcctaagcgtttgccatcaacgttggcgaagtttccttatgtcaatggtgccctatttgctgaaccgttggcctcggagtacttcgactaccagatgcgcgaagcattgcttgctgcctgcgacttcgactggtcgaccattgacgtctccgtctttggttcgttgttccaattggtgaaatcgaaggaagcgcgccgcagcgacggcgaacactacacgtctaaggccaacatcatgaagaccatcggcccgctgtttttggacgagctgagggctgaggccgataagttggtgtcttctccgtcgacgtcggtggccgcattagagcgcttccgcgactccctgtctgagctggtattcgctgatatggcttgtggttctggaaacttcctgcttctggcgtatcgggagttgcgccggattgaaaccgacatcattgtcgctatacgccagcgccgcggtgaaacgggcatgtcgttgaatattgagtgggagcagaaactgtccattgggcagttctacggcattgagctgaattggtggcctgccaagattgctgagactgccatgttcctagttgaccatcaggccaacaaggagcttgccaacgctgtgggtaggcctccggagcggttgccgattaagattaccgcgcacattgtgcacggcaatgccctgcagcttgattgggcagacatactctcggcttctgccgccaagacgtatatcttcggtaacccgccgtttttggggcatgcgacgagaactgctgaacaagctcaagaactccgagacttgtggggcactaaggacatttcacgcttggactacgtcaccggctggcatgcaaagtgcttggatttctttaagtcccgagagggtcgttttgcgtttgtcaccaccaattcaattactcaaggtgatcaagttccacggctatttgggcctatcttcaaagcagggtggcgtattcgtttcgctcaccgcacgtttgcgtgggactctgaagcacccggtaaagctgctgttcactgcgtcattgttggcttcgataaggagagtcaaccacgtccacgtctgtgggattatcccgatgtaaagggcgagccagtctcagtggaagtaggccagtccattaatgcctatttagtagacggccctaatgttcttgtcgataaatcccggcatcctatttcgtcggaaatatcgcccgcaacttttggaaatatggcgcgagatggcggcaaccttctagttgaggtcgacgaatacgacgaggttatgagtgaccccgtagcggcaaagtatgttcgccctttccggggtagtcgagagctaatgaacggcttagatcggtggtgtctatggcttgtagatgtagcaccgtcagacattgcccagagtccggttctgaaaaagcgtctagaagcggttaagtcttttcgagccgacagtaaagcggcaagtacacggaaaatggctgaaactccgcacttattcggccagcggtcgcaaccggatactgattacctttgcctgccgaaggtagtaagcgaacgccgctcgtatttcaccgtacaaaggtatccatcaaacgtaatcgcttctgacctagtattccatgctcaagatccagacggcctgatgtttgcgctagcgtcgtcgtcgatgttcattacgtggcagaaaagcatcggaggacgactcaagtctgatctccgttttgctaacactttgacgtggaatactttcccagtgccagaactcgacgagaagacgcggcagcgaattattaaagcgggcaagaaggtgctcgacgcccgcgcgctgcacccagaacgctcgctggccgagcactacaacccactcgcgatggcaccggaactcatcaaagcgcatgatgcgctcgaccgcgaggtggataaagcgtttggcgcgccacgaaagctgacaactgttcggcagcgccaggagctattgtttgccaattacgaaaaactcatctcacaccagccctag

SEQ ID NO: 12 - protein, 952 aa, Corynebacterium striatum M82B
MVMAPTTVFDRATIRHNLTEFKLRWLDRIKQWEAENRPATESSHDQQFWGDLLDCFGVNARDLYLYQRSAKRASTGRTGKIDMFMPGKVIGEAKSLGVPLDDAYAQALDYLLGGTIANSHMPAYVVCSNFETLRVTRLNRTYVGDSADWDITFPLAEIDEHIEQLAFLADYETSAYREEEKASLEASRLMVELFRAMNGDDVDEAVGDDAPTTPEEEDERVMRTSIYLTRILFLLFGDDAGLWDTPHLFADFVRNETTPESLGPQLNELFSVLNTAPEKRPKRLPSTLAKFPYVNGALFAEPLASEYFDYQMREALLAACDFDWSTIDVSVFGSLFQLVKSKEARRSDGEHYTSKANIMKTIGPLFLDELRAEADKLVSSPSTSVAALERFRDSLSELVFADMACGSGNFLLLAYRELRRIETDIIVAIRQRRGETGMSLNIEWEQKLSIGQFYGIELNWWPAKIAETAMFLVDHQANKELANAVGRPPERLPIKITAHIVHGNALQLDWADILSASAAKTYIFGNPPFLGHATRTAEQAQELRDLWGTKDISRLDYVTGWHAKCLDFFKSREGRFAFVTTNSITQGDQVPRLFGPIFKAGWRIRFAHRTFAWDSEAPGKAAVHCVIVGFDKESQPRPRLWDYPDVKGEPVSVEVGQSINAYLVDGPNVLVDKSRHPISSEISPATFGNMARDGGNLLVEVDEYDEVMSDPVAAKYVRPFRGSRELMNGLDRWCLWLVDVAPSDIAQSPVLKKRLEAVKSFRADSKAASTRKMAETPHLFGQRSQPDTDYLCLPKVVSERRSYFTVQRYPSNVIASDLVFHAQDPDGLMFALASSSMFITWQKSIGGRLKSDLRFANTLTWNTFPVPELDEKTRQRIIKAGKKVLDARALHPERSLAEHYNPLAMAPELIKAHDALDREVDKAFGAPRKLTTVRQRQELLFANYEKLISHQP

SEQ ID NO: 13 - DNA, 2814 bp, Neisseria meningitidis Z2491
atgaaaaccctgctccaactccaaaccgccgcacaaaacttcgccgcctactacaaagaccaaaccgacgaacgccgcgagaaagacaccttctggaacgaatttttcgccattttcggcatcgaccgcaaaaacgtcgcccacttcgaataccccgtcaaagaccctgccgacaacacccaattcgtcgatatattttgggaaggcatcttccttgccgaacacaaatccgccaacaaaaacctgaccaaggccaaagagcaggcggaacgttatttacaggaaatcgggcgcaccaagccctccgcgctgcccgaatattacgccgtcagcgattttgcccatttccacctttaccgccgcgtacctgaagaaggcgcagaaaaccaatggcagttccctttggaagaattgcctgaatacatcacgcgcggcgttttcgacttcatgttcggcatcgaagccaaagtccgccaaattcaagaagaagccaacattcaagcggcggcgaccatcggcaggctgcacgacgcgctcaaagaagaaggcatttacgaagaacacgagctgcgcctcttcatcacgcgcctgcttttcctcttttttgccgacgacagcgccgttttccggcgcaactaccttttccaagactttttagaaaactgcaaagaagccgacacgctcggcgacaagctcaatcaactctttgaatttctcaacacacccgaccaaaagcgcagcaagacccaaagcgaaaaatttaaaggtttcgaatacgtcaacggcggtcttttcaaagaacgcctgcgcactttcgacttcactgccaagcagcaccgcgccttaatcgactgcggcaatttcgactggcgcaacatcagtccagaaatcttcggcacgctcttccaatccgtcatggacgcgcaagagcggcgcgaagcgggcgcgcactacaccgaagccgccaatatcgacaaagtcatcaacggcctttttttagaaaacctgcgtgccgaatttgaagccgtcaaagccctcaaacgcgacaaagccaaaaaactcgccgccttctaccaaaaaatccaaaacctgcaattcctcgaccctgcctgcggctgcggcaacttccttatcgtcgcctacgaccgcatccgcgcccttgaagacgacatcatcgccgaagccctcaaagacaaagcagacggcctgttcgacagcccgtccgtccaatgccgtctgaaacagtttcacggcatcgaaatagacgaatttgccgtcctcatcgcccgcaccgccatgtggctcaaaaaccaccaatgcaacatccgcacacaaatccgcttcgacggcgaagtcgcctgccatacgctgccgctcgaagacgccgccgaaatcatccacgccaacagcctccgcacaccttggcaggcggcggactacatcttcggcaatcccccctttatcggctcgacctaccaaaccaaagagcagaaaaacgacctcgaaagcatctgcggccatatcaaaggctacggcctgttggattacgtctgcaactggtacgtcaaagccgcaggcatcatggcgcagcatccccaagttcagacggcatttgtttccaccaattccatctgccaaggccagcaggtcgaaatcctctggggcagccttttaaaccaaggcatcgaaatccactttgcccaccgcaccttccaatggacgagccaagccgcaggcaaagccgccgtccactgcatcatcgtcggcttccgccaaaagccgccaatgccgtctgaaaaaaccctctacgactatcccgacatcaaaggcgaacccgaaaaacacgccgtagccaacatcaatccttatctgatcgatgcgcccgatttgattatcgccaagcgcagccgtcccatacattgcgaacctgatatggtcaacggaagcaaaccgaccgaaggcggcaaccttatcctttcaaccgccgaaaaagatgccctgattgccgccgaacccttggcggagcaatacatccgcccctttatcggcgcggatgagtttctcaacggcaaaacccgttggtgcctgtggtttcacggcgtatccgatgtcaaacgcaaccacgacctgaaacaaatgccccaagttcaagcccgtattcaggcggtcaaaaccatgcgcgaagccagcagcgacaaacaaactcaaaaagatgcagcaaccccgtggctttttcaaaaaatccgccagccttcagacggcaattatctgattattccgagcgtgtcgtctgaaagccgccgtttcatccccatcggttatctgtcgtttgaaacagttgtcagcaatctggcatttatccttccaaacgccaccctctaccacttcggcatcctcagctccaccatgcacaacgcctttatgcgtaccgttgcaggtcgtctgaaaagcgattatcgctactctaataccgtcgtgtacaacaacttccccttccccgaaagctgccggttgccgtctgaaaacgaccgccccgacccgctccgcgccgccgtcgaagccgccgcccaaaccgtcctcgacgcgcgcggacaataccgccgagaagcgcaggaagccggtttgcccgagccgaccctcgccgaactctatgcgcccgacgcaggctataccgccctcgacaaagcccacgccaccctcgacaaggcagtcgataaagcctacggctacaaaacaggcaaaaataccgacgacgaggcagaacgcgtcgccttcctgttcgagctgtaccgcaaggcggcggcaattgcgtag

SEQ ID NO: 14 - protein, 937 aa, Neisseria meningitidis Z2491
MKTLLQLQTAAQNFAAYYKDQTDERREKDTFWNEFFAIFGIDRKNVAHFEYPVKDPADNTQFVDIFWEGIFLAEHKSANKNLTKAKEQAERYLQEIGRTKPSALPEYYAVSDFAHFHLYRRVPEEGAENQWQFPLEELPEYITRGVFDFMFGIEAKVRQIQEEANIQAAATIGRLHDALKEEGIYEEHELRLFITRLLFLFFADDSAVFRRNYLFQDFLENCKEADTLGDKLNQLFEFLNTPDQKRSKTQSEKFKGFEYVNGGLFKERLRTFDFTAKQHRALIDCGNFDWRNISPEIFGTLFQSVMDAQERREAGAHYTEAANIDKVINGLFLENLRAEFEAVKALKRDKAKKLAAFYQKIQNLQFLDPACGCGNFLIVAYDRIRALEDDIIAEALKDKADGLFDSPSVQCRLKQFHGIEIDEFAVLIARTAMWLKNHQCNIRTQIRFDGEVACHTLPLEDAAEIIHANSLRTPWQAADYIFGNPPFIGSTYQTKEQKNDLESICGHIKGYGLLDYVCNWYVKAAGIMAQHPQVQTAFVSTNSICQGQQVEILWGSLLNQGIEIHFAHRTFQWTSQAAGKAAVHCIIVGFRQKPPMPSEKTLYDYPDIKGEPEKHAVANINPYLIDAPDLIIAKRSRPIHCEPDMVNGSKPTEGGNLILSTAEKDALIAAEPLAEQYIRPFIGADEFLNGKTRWCLWFHGVSDVKRNHDLKQMPQVQARIQAVKTMREASSDKQTQKDAATPWLFQKIRQPSDGNYLIIPSVSSESRRFIPIGYLSFETVVSNLAFILPNATLYHFGILSSTMHNAFMRTVAGRLKSDYRYSNTVVYNNFPFPESCRLPSENDRPDPLRAAVEAAAQTVLDARGQYRREAQEAGLPEPTLAELYAPDAGYTALDKAHATLDKAVDKAYGYKTGKNTDDEAERVAFLFELYRKAAAIA

SEQ ID NO: 15 - DNA, 2781 bp, Corynebacterium diphtheriae
atgtcatcgagttctccaagtgaaaagaaactagccgccaagctatttgctaataagtgggcagaccgtggcaatgagaaaagcgacactcacagtttctggttggagcttcttcgtgatgttgtaggtatgcaagatgtgactaccaacgtgcgattcgaatcgcgcacgagtcaacgcggctacatcgatgtggtgatccaagacgccaaaactttcattgaacaaaaatccatcgatgttagtttggacaaagctgatatccgtcaggggcgagttgtcactgcttttagacaagcactgaattacgccaacactatgccgaacaaactgcgacctgactacattattacgtgtaatttcgcagagtttcgtattcatgacttaaataaggtgaatgcggaaactgactatatttcctttaccttggcagaattgcctgaccaaatccatcttctagattttctcatcgacccacaaaaatctcgtgctgttcgtgaagaaaaagtgtcgatggatgctggcacactcgtcggcaagctttacgacgccctgcgtgatcagtatttagaccccaacagtgatgcgagccagcactccctcaacgttttgtgcgtgcgccttgtattttgtttgtttgctgaagacgccggcctctttgaaaaggatgcgttttatcgttatcttgacggattacgcgccgatcaagttcgcgtcgcgctgagagatttgttcgaagtactcaatacaccagttgattcacgtgacccttatctttctgaacagcttaaaaacttcccttatgtcaacggtggtttattcgccaaagtcgagcagatccctaatttcactgatgaaattcttgacctattagttcatgaggtatcggagaaaactaactgggccgaaatctcgcctacaatctttggcggtgtttttgaatccaccctcaacccagaaactcgcgcccgtggaggcatgcattacacgagtcccgaaaacatccataaggtgattgacccgctgtttcttgactctctcaaggcagagctagattccatccttaacgcatcagggataactgcaaacaagcgcaagaaacaactcgaggcattccacaccaagatctcagagctaaaatttttcgaccctgcctgcggttcgggaaacttcctcacagaaacctatatccacctgcgcaagatcgaaaacaagatcctttcagagcttgccggcgaccaaacccagctcggctttagcaacgtcactctcaaggtcagcttggaccagttctacggcatcgagatcaatgatttcgccgtctccgtcgcctccaccgccctatggattgcgcagctccaggccaacatcgaggccgaatcgatcgtcaccgcaaacatcgaaagtcttccgcttcgcgacgccgcccacatccacctcggtaatgcgctgcgcaccgactgggcttcggtactcgcgcctgaacagtgcaattacattattggaaatccgccgtttttaggctactcgcggcttgacgacgctcaaaaggaagaccgcaaggccatcttcggcaagaatggcggtgtgctcgattacgtagcgtgctggcaccgcaaagccgccgaatatatgcacggaacggatgctgaagccgcgctcgtttccaccaattcgatctgccaaggccagcaagtcactccgctgtggaagccgcttttcgacgccgggatccacatcaacttcgcccaccgcactttcgtgtggagcaacgaggcagcagatcaggcgcatgtcttatgtatcatcgtcgggttttcctacatcgatcgaccagtcaagcaggcgtggacctaccggaagaacgaggtggaatactcggagcctgtacatttgaacggttacttggcagatgccccggatgcgttcctgacacgcaggtcaaagccgatttcggatgtgctggaaatggctcagggattcaagcccgccgatggtggacatctcttgctcactcaagaagaacgagacgaactccttgcaaaagaaccactagctgcgccgtggattcgaaagttctccatgggcgccgaattcatcaacggcaaggaccgctattgcctatggttgccggaaattacaggcgttgagctaaagagattgcctctcgttcgcgcgcgaattgacgcatgccgtgagtggaggcttgaacaaatcaaaactggagatgcatacaaattgtcagaccggccacacctactgcggccaaccagcaggtttaaggacggaacctacatcggcatcccaaaggtttcttcagagcgacggaagtatgtaccgtttgcttttgtgacagatggaatgattcctggcgacatgctctacttcgtccctacggattctctatttgtgtttggggttctcgtttcacaattccaaaacgcctggatgcgtgtagtggcaggccgtctcaagagcgactaccgctatggcaacaccactgtctacaacaacttcgttttccccgaggtagatgattcagtgcgagtggacgtcgaaaagcgtgctcaggcggtgatcgacgcacgctctctttaccccgaagcgacgcttgctgacatgtatgatcccgacaatgacttcctctaccccgagctcatgaaggcccaccgcgagctagaccgcgctgtcgagatggcttatggcgtggacttcggtggcgacgagcagcagatagtggctcacctcttcaagctgtacaacgagaaagtagagaaatga

SEQ ID NO: 16 - protein, 926 aa, Corynebacterium diphtheriae
MSSSSPSEKKLAAKLFANKWADRGNEKSDTHSFWLELLRDVVGMQDVTTNVRFESRTSQRGYIDVVIQDAKTFIEQKSIDVSLDKADIRQGRVVTAFRQALNYANTMPNKLRPDYIITCNFAEFRIHDLNKVNAETDYISFTLAELPDQIHLLDFLIDPQKSRAVREEKVSMDAGTLVGKLYDALRDQYLDPNSDASQHSLNVLCVRLVFCLFAEDAGLFEKDAFYRYLDGLRADQVRVALRDLFEVLNTPVDSRDPYLSEQLKNFPYVNGGLFAKVEQIPNFTDEILDLLVHEVSEKTNWAEISPTIFGGVFESTLNPETRARGGMHYTSPENIHKVIDPLFLDSLKAELDSILNASGITANKRKKQLEAFHTKISELKFFDPACGSGNFLTETYIHLRKIENKILSELAGDQTQLGFSNVTLKVSLDQFYGIEINDFAVSVASTALWIAQLQANIEAESIVTANIESLPLRDAAHIHLGNALRTDWASVLAPEQCNYIIGNPPFLGYSRLDDAQKEDRKAIFGKNGGVLDYVACWHRKAAEYMHGTDAEAALVSTNSICQGQQVTPLWKPLFDAGIHINFAHRTFVWSNEAADQAHVLCIIVGFSYIDRPVKQAWTYRKNEVEYSEPVHLNGYLADAPDAFLTRRSKPISDVLEMAQGFKPADGGHLLLTQEERDELLAKEPLAAPWIRKFSMGAEFINGKDRYCLWLPEITGVELKRLPLVRARIDACREWRLEQIKTGDAYKLSDRPHLLRPTSRFKDGTYIGIPKVSSERRKYVPFAFVTDGMIPGDMLYFVPTDSLFVFGVLVSQFQNAWMRVVAGRLKSDYRYGNTTVYNNFVFPEVDDSVRVDVEKRAQAVIDARSLYPEATLADMYDPDNDFLYPELMKAHRELDRAVEMAYGVDFGGDEQQIVAHLFKLYNEKVEK

SEQ ID NO: 17 - DNA, 2847 bp, Arcanobacterium pyogenes
atgctctctgatcctgtctttgaccgtgccaccatccgccataaactcattgagttcaaaatccgctggcgcggccatatcgaccagtggaaagcagaaaaccgccccgccaccgagtccagccacgatcaacagttctggggtgacctcctagcctgcttcggcgtcaacgcccgcgacctttacctgtatcagcgcagcgcgaaacgagcctccaccggccacaccggcaagattgacatgttcatccccggcaaagtcatcggcgaggccaagtccctcggtatcgacctggacaaggctcacgagcaagcactcgactacctgctcggcggcaccattccgaactcacaaatgccggcctatgtcctctgctccaacttcgagaccctgcgcatcacccgccttaaccgcgactacgtcggcgactctgcagaatgggacgttaccttcgacctggacgaaatcgacgagcatctggaacagctcgcgttcctggcggactatgagacctcggcctatcacgaggaagaacaagcctcccttgaggcctcacgcctgatggtcgagctgttccgcgccatgaacggcgacgaggcagacgaagccgtgggcgatgaagccccaaccaccccggaggaagaagacgaaagggtcatgcgcacctcggtctacctaacgcgcatcctcttcctccttttcggcgacgatgcaggcctgtgggacaccccgcacctgtttacgacgttcgtgcgcaacgaaaccaccccggaatctctcggacctcagctcaacgaacttttccgagtcctcaacaccccggaggacaagcggcctaagcgcttgcccggcaccttggcgaaattcccctacgtcaacggcgcaatcttcgccgaacagctcgaccctgaatacttcgactacgccatgcgcgaagccctgctcaacgcctgcgacttcgactggtcaaaaatcgacgtgtccgtcttcggctcactgttccagctggttaagtcgaaagaagcccgccgtggcgatggtgagcactacacctcgaagaccaacatcctcaagaccatcggaccgctcttcctcgacgagttgcgtgcccaggctgacaagctggtctccaaccccgccaccccggtgcgcaagttagaagaattccgcgactcactggctgcccatattttctgcgacccggcctgtggtgcgggaaacttcctgctcaccgcctataaagaactgcgccgtattgaaacggaccttatcgtggctatccgtcagcgccgtggcgagacgggtatgtcgctaaatattgagtgggagcagaaactgtcgattgggcagttctacggatttgagctgaactggtggccggcaaagattgcagagacggcgatgttcctggtggatcatcaggcgaataaggagttggcgaatgcggtggggcgtccgccgcagcgtttgcctattacgattaccgcccacatcgtccacggaaacgctctcgccctggactggacggaagcgctgcccaaagcagtgggggagacgtttatctttggcaacccaccatttatcggtcaagatacgcgcacaaaacagcagctcgaggaaatgaaagctgtatggagacgtaaaaacatctcgagattggactacgtcacgtgttggcacataaaaagccttgaccttttcagtacccgtaacggacggttcgctttcgtaacaactaactcgattacccaaggcgaacaagtgccgcttttattcggccccatcttcgcagcaggttggcgtatccgcttcgcccatcgcacattctcatgggattccgatgctcccggtaaagcctcagtccactgcgtcatcgtcggtttcgaccgtgcacacgaacctcgcccccagctctgggattacccgaatgtcagcagtgcccccgtggctgtgcctgtggagcgcgtgattaatgcttacctcgtcgacggccctaatgtccttgtccaaaagatgacttcgcccatctcctgcgagattaaacccgcagttctaggcgcaatggcaaaagacggaggtggcttgatagttgaagcccaggacgtgcaagaagctttggacgatccgatagcggcaaagtacctacgtccgtacgttggctcgcgagaacttgttcgcggccttagtcggtggtgtctctggatggtcgatctcgaccccgccgacgttcaggcaagtacttttctgcgttcacgaattgaacaagtacgcgcctacagaacaacgtcctcggctcctactacacggagcatggcaaagattcctcatcttttcgcacaacgttatcggccacaaacagatttcctttgcgttccatccgttgttagcgagaaccggccatacttcacagctgcggatattgaggaaggaacagttgtctccagccttgcgtttgcggttgaagattctgataggtcacagttcgcgttgatttcttcgtcaatgttcattacttggcaaaagatgattggaggaaggctagaatctcgcctgcgttttgcgaacacactgacgtggaacacgttccccgtaccagaactcgatgagaagacgcgcaagcggattattaaggctgggcagaaagtactcgccgcgcgcgcactgcacccggagcgttccctcgcggagcactacaacccgctggctatgacaccagaactggtgaaggcgcatgacgcgctcgaccgggaagtggataaagcaatgggggcggcgcgcaagctcacttcggagcggcagcgccaggagctactgtttgccaattacgcgaaactcaccaacaactag

SEQ ID NO: 18 - protein, 948 aa, Arcanobacterium pyogenes
MLSDPVFDRATIRHKLIEFKIRWRGHIDQWKAENRPATESSHDQQFWGDLLACFGVNARDLYLYQRSAKRASTGHTGKIDMFIPGKVIGEAKSLGIDLDKAHEQALDYLLGGTIPNSQMPAYVLCSNFETLRITRLNRDYVGDSAEWDVTFDLDEIDEHLEQLAFLADYETSAYHEEEQASLEASRLMVELFRAMNGDEADEAVGDEAPTTPEEEDERVMRTSVYLTRILFLLFGDDAGLWDTPHLFTTFVRNETTPESLGPQLNELFRVLNTPEDKRPKRLPGTLAKFPYVNGAIFAEQLDPEYFDYAMREALLNACDFDWSKIDVSVFGSLFQLVKSKEARRGDGEHYTSKTNILKTIGPLFLDELRAQADKLVSNPATPVRKLEEFRDSLAAHIFCDPACGAGNFLLTAYKELRRIETDLIVAIRQRRGETGMSLNIEWEQKLSIGQFYGFELNWWPAKIAETAMFLVDHQANKELANAVGRPPQRLPITITAHIVHGNALALDWTEALPKAVGETFIFGNPPFIGQDTRTKQQLEEMKAVWRRKNISRLDYVTCWHIKSLDLFSTRNGRFAFVTTNSITQGEQVPLLFGPIFAAGWRIRFAHRTFSWDSDAPGKASVHCVIVGFDRAHEPRPQLWDYPNVSSAPVAVPVERVINAYLVDGPNVLVQKMTSPISCEIKPAVLGAMAKDGGGLIVEAQDVQEALDDPIAAKYLRPYVGSRELVRGLSRWCLWMVDLDPADVQASTFLRSRIEQVRAYRTTSSAPTTRSMAKIPHLFAQRYRPQTDFLCVPSVVSENRPYFTAADIEEGTVVSSLAFAVEDSDRSQFALISSSMFITWQKMIGGRLESRLRFANTLTWNTFPVPELDEKTRKRIIKAGQKVLAARALHPERSLAEHYNPLAMTPELVKAHDALDREVDKAMGAARKLTSERQRQELLFANYAKLTNN

SEQ ID NO: 19 - DNA, 2898 bp, Silicibacter pomeroyi DSS-3
atgacgccccaagatttcatcaccaaatggcgcaacaccgaactcaaggaacggtccgcatcccagtcgcatttcattgacctgtgccgccttctggacatcgaagacccgacaaccgcagaccccaagggcgagtggttcaccttcgaaaaaggagcgtccaagacaagtggcggcgaaggctgggcggacgtctggcgcaaggattgctttgcgtgggaatacaagggcaagcgcgccaatctggacaaggcgtttgaccagctcttgcaatacgccatcgcgctggagaacccgccgcttctgatcgtgtcggacatggatgtgatacgcatccacaccaactggaccaacacggtgcagcaggtgcacacccttacactggacgacctcaaggacgccgccaaccgtgacaagctacgcaacgcttttctcaaccccgacgtcttcaagccctccaagacccggcaacttgttaccgaacaggcggcacagaactttgccaaccttgcccagcgtctccgggaacgtggccacgacgcgcaacaggtggcgcatttcgtcaaccgtctggtgttctgcatgtttgccgaggatgtggagcttttgccgaacaagatgttcgagcggatgatcaaggccgcgcgccctgaccccgccagctttgccatccacgccaaggcgctctttgcagctatgaaagacggcgggcttgtgggcttcgaaaaggtggactggttcaacggcggcctgttcgacaatgacgacgtgctgccgctggaatgggaagacttagacgacctcattcgcgcggcacatctggactggtccgacattgacccgtccatccttggcaccttgttcgaacgcgggttggacccggccaagcgcagccagttgggcgcgcattacaccgaccgcgacaagatcatgcagatcgtgaacccggtcattgtcgaaccgctcttggccgaatgggccgaggtgaaagcccagatcgaagacctgatcgacaaagcccccaaggcgacgaaggacaagcttctcagcacgtcgcagaaggccgcccgcacccgcgcgctggacaaggccgaggcgctgcaccaagcgtttctggaccggctcaaggcgttccgtgtgctggacccggcctgtgggtctggcaacttcctctacatcgcgcttctggaactcaagaacatcgaacatcgggtgaacctagaggccgaggcgctgggcctgccccgagggttcccgcaaatcggccccgaggttgtgctgggcatcgaactcagcgcctatgcggcggaactggcccgcgtgtcagtctggattggcgaaatccaatggatgcgccgcaacggattcgaggcggcgaagaacccgatcttgcggtcccttaagacgattgagaaccgggacgcggtgttgaacccggacgggacgcgggcggactggccgaaggcggatgtggttgtcgggaaccccccgtttttgggcgtctacaaaatgggagaagaactaggggaagattacacaattgcattgcgcgatgcttggccggaaatgccgggagccgcagaccttgttacctattggttcgccaaagcttggtcacagatgcaatgcggagacctaagtcgtgctggacttgtggcaacgaactctattcgcggtggtgcaaataggactgtcctaaaaccgattgccgaacatggcggaatttttgatgcatggtcggacgaagcatggacagtagagggcgcagcagtgcgtgtatctatgatttgctttggaagcaaactgccgtctcaccccaagttaaatggcaaagttgtggataaaattctttctgatttaactgcaaacgctgccgggtttgatcttacaaaatcatctcgaatttcagaaaataaaggtgtttgcatccggggcattgaaaccggcggtccatttgaattttcgcaggcggatttcgaagcacttgctacaaagcctctgaatcccaacgggctacccaacacacgagttatccggagaattctaaatgggaacaatattctgaagcggcaaccagaacgttatgcgatagacttctctgacttccgcacgaaggaagaggccgcattgttcgaagcggtctattcatggcttgaacaagcctacgaaagctatgagcggaaatcgaagcgccggattgtaagacgtcaggactggtggctgcatcgaagatcaggagcagcgctcaaaaatgcggtaagtagactttcccgatttattgttacaccgcgtgttggaaaacacagaatattcgtatggcttgactcaaatgcacttgcagatagcgccacgttcatagtggcccgcgacgatgaaaccaccttcggcattctgcattccagttttcatgaactctggtcactgcgtatgggcactttccttggggtgggtaacgacccccgctacaccccctctaccaccttcgaaacctttcccttccccgaaggcctcacccccaacatccccgccgacgagtatgccgatgccccccgcgccatcaaaatcgccgccgccgccaagcgcctaaacgagtttcgggaaaactggctcaaccccgccgatctggtggaccgcgtgccagaggtcgtttccggctaccccgaccgcatccttcccaagaacgacgccgccgccaaggaactcaagaaacgcaccctgacgaacctctacaacgcccgccccgcatggctcgaccacgcccacaaggcgttagacgaagcggtggccgaagcctacggctggggcgacgactggcgcgcgggcgtgctgaccgaagacgaaatcctggcccgcctgttcaagctcaaccaagagcgcgcagcgaaggagaaagcatga

SEQ ID NO: 20 - protein, 965 aa, Silicibacter pomeroyi DSS-3
MTPQDFITKWRNTELKERSASQSHFIDLCRLLDIEDPTTADPKGEWFTFEKGASKTSGGEGWADVWRKDCFAWEYKGKRANLDKAFDQLLQYAIALENPPLLIVSDMDVIRIHTNWTNTVQQVHTLTLDDLKDAANRDKLRNAFLNPDVFKPSKTRQLVTEQAAQNFANLAQRLRERGHDAQQVAHFVNRLVFCMFAEDVELLPNKMFERMIKAARPDPASFAIHAKALFAAMKDGGLVGFEKVDWFNGGLFDNDDVLPLEWEDLDDLIRAAHLDWSDIDPSILGTLFERGLDPAKRSQLGAHYTDRDKIMQIVNPVIVEPLLAEWAEVKAQIEDLIDKAPKATKDKLLSTSQKAARTRALDKAEALHQAFLDRLKAFRVLDPACGSGNFLYIALLELKNIEHRVNLEAEALGLPRGFPQIGPEVVLGIELSAYAAELARVSVWIGEIQWMRRNGFEAAKNPILRSLKTIENRDAVLNPDGTRADWPKADVVVGNPPFLGVYKMGEELGEDYTIALRDAWPEMPGAADLVTYWFAKAWSQMQCGDLSRAGLVATNSIRGGANRTVLKPIAEHGGIFDAWSDEAWTVEGAAVRVSMICFGSKLPSHPKLNGKVVDKILSDLTANAAGFDLTKSSRISENKGVCIRGIETGGPFEFSQADFEALATKPLNPNGLPNTRVIRRILNGNNILKRQPERYAIDFSDFRTKEEAALFEAVYSWLEQAYESYERKSKRRIVRRQDWWLHRRSGAALKNAVSRLSRFIVTPRVGKHRIFVWLDSNALADSATFIVARDDETTFGILHSSFHELWSLRMGTFLGVGNDPRYTPSTTFETFPFPEGLTPNIPADEYADAPRAIKIAAAAKRLNEFRENWLNPADLVDRVPEVVSGYPDRILPKNDAAAKELKKRTLTNLYNARPAWLDHAHKALDEAVAEAYGWGDDWRAGVLTEDEILARLFKLNQERAAKEKA

SEQ ID NO: 21 - DNA, 2871 bp, Deinococcus radiophilus R1
atgcctcagaccgagaccgcgcagcgtatggaagacttcgttgcctactggcgcaccctgaaaggggacgagaagggcgaaagtcaggtatttctggaccggctctttcaggcctttgggcacgccggatacaaggaagcgggcgcggaactggagtaccgggtcgccaagcagggcggcggcaaaaaattcgctgacctgctgtggcggccccgcgtgctgatagagatgaaaaagcgcggcgagaaactggcgaaccactaccagcaggccttcgactactggctcaagctggtgccggaccgcccacgttacgccgtgctgtgcaatttcgacgagctgtgggtctacgacttcaatcagcagctcgacgagccgatggaccggctgcggatagaagaactgcctgagcggtacacggtgctgaacttcatgtttgagcaggaaagggcgccgctgttcggcaacaaccgggtggacgtaacccgcgaggccgccgacagcgtagcgaaggtgctcaacagtgtgattgcccgtggtgaagaccgcgcccgcgctcagcgtttcctcttgcagtgcgtcatggcgatgttcgccgaggacttcgagttgattccgcgtggcttttttaccgaattggccgacgacgccagggcaggccggggaagcagcttcgacctcttcggcgggctgttccggcagatgaatacctccgaacgggcacggggcgggcgttttgcgcccattccgtatttcaacggcgggctgttccgcgccgtggaccccattgaacttaaccgcgatgagctttacctgctgcacaaagccgcgctggaaaacaactgggccaggattcagccgcagattttcggggtgctgtttcagagcagcatggacaagaaagagcagcacgccaagggggcgcactacaccagcgaggccgacatcatgcgggtggtgttgcccaccatcgtcaccccgtttcagcggcaaatcgaggcggcgaccacgcaaaaggaactgcgggccattctggacgaactcgccagctttcaggtgctcgaccccgcgtgtggcagcggcaacttcctgtatgtcgcctaccgcgaactgcgccgcctggaagcccgcgccctgctgcggctgcgtgacctctccgcaccggggaccgccctgccgcctgcccgcgtgagcatccggcagatgcacgggctggaatacgaccccttcggcgtggaactcgccaaagtgaccctcacgctcgccaaagaactcgccatccgtgagatgcacgacctgctgggcaacaccggcctggacttcgaccagccgctgccgctggacaacctcgacgaccgtatcgtgcagggcgacgccctctttaccccgtggccccgtgtggacgccatcgtcggcaaccccccgtttcagagcaaaaacaagttgcagcgcgagatgggcgcggcctatgtcaaaaagctccgtgcccactaccccgacgtgccgggccgcgccgactactgcgtctactggattcgcaaggcgcatgaccaactgggcagcggccagcgggcgggtctggtgggcaccaacaccattcgtcagaacgacagccgtgtcggggggctggattatgtcgtgcagcacggcggcaccatcaccgacgccgtgggcacgcaagtctggtccggcgacgccgctgtgcatgtcagcatcgtcaactgggtcaaggggccagccgaaggccccaagcatctggcgtggcaggtgggcgaccaccgcaccagcccctggcaaagcaccgagttgcccgtcatcaactctgccctgtctgccggaaccgatgtcacgcaggcgcaaaagctgcgcgtcaacatgaacagcggcgcgtgctaccagggccagacccacggccacaaaggctttttgctggacggtctggaagccgggcagatgctcagcgccgagcgcaaaaacgccgaggttatttttccgtacctcacgggtgatgaactgctccgcaccagcccgccgcacccgacccgttatgtcattgattttcagccgcgtgacgtgttcggcgcgagggcctacaaattgccctttgcccgcatagaacgcgaagtgctgcctacgcgccaggccgccgccgccgaggaagaagcccgcaacgccgaagtgctggccgccaacccaaaggccaagaccaacaaacaccaccgcaatttcctgaatcagtggtgggcactgtcgtatgggcgcagtgaaatgattgagaaaatttcatcactgagccgttatattgtctgctcgcgcgttaccaaaaggcaagtatttgagtttctagataatggtatccgtcctagtgacggtcttcaaattttcgcctttgaagatgattattcatttggagtcatccaaagttctgtccattggcagtggttaattgcacgtgggggaacattaacggcccgtcttatgtacacctccgataccgttttcgacaccttcccctggcctcaagacccgacactggcgcaggtgcgggcggtggcggcggcagcggtgaagctgcgggaactgcggaacaaggtgatgcgcgagcagggctggagcctgcgcgacctgtaccggacgctggacatgccgggcaaaaacccgctgcgtgacgctcaggaacggctggacgcggcggtgagtgcggcttatggcctgccagcgggggcggacatgttggactttttgctggccctgaacgcaraagtggcggcggcggaagcgcggggcgcggcggtgacggggccgggcctgcctgcgggcctgaacacggcggacttcgtgacggcagatgcggtgcggcctctgggctga

SEQ ID NO: 22 - protein, 956 aa, Deinococcus radiophilus R1
MPQTETAQRMEDFVAYWRTLKGDEKGESQVFLDRLFQAFGHAGYKEAGAELEYRVAKQGGGKKFADLLWRPRVLIEMKKRGEKLANHYQQAFDYWLKLVPDRPRYAVLCNFDELWVYDFNQQLDEPMDRLRIEELPERYTVLNFMFEQERAPLFGNNRVDVTREAADSVAKVLNSVIARGEDRARAQRFLLQCVMAMFAEDFELIPRGFFTELADDARAGRGSSFDLFGGLFRQMNTSERARGGRFAPIPYFNGGLFRAVDPIELNRDELYLLHKAALENNWARIQPQIFGVLFQSSMDKKEQHAKGAHYTSEADIMRVVLPTIVTPFQRQIEAATTQKELRAILDELASFQVLDPACGSGNFLYVAYRELRRLEARALLRLRDLSAPGTALPPARVSIRQMHGLEYDPFGVELAKVTLTLAKELAIREMHDLLGNTGLDFDQPLPLDNLDDRIVQGDALFTPWPRVDAIVGNPPFQSKNKLQREMGAAYVKKLRAHYPDVPGRADYCVYWIRKAHDQLGSGQRAGLVGTNTIRQNDSRVGGLDYVVQHGGTITDAVGTQVWSGDAAVHVSIVNWVKGPAEGPKHLAWQVGDHRTSPWQSTELPVINSALSAGTDVTQAQKLRVNMNSGACYQGQTHGHKGFLLDGLEAGQMLSAERKNAEVIFPYLTGDELLRTSPPHPTRYVIDFQPRDVFGARAYKLPFARIEREVLPTRQAAAAEEEARNAEVLAANPKAKTNKHHRNFLNQWWALSYGRSEMIEKISSLSRYIVCSRVTKRQVFEFLDNGIRPSDGLQIFAFEDDYSFGVIQSSVHWQWLIARGGTLTARLMYTSDTVFDTFPWPEDPTLAQVRAVAAAAVKLRELRNKVMREQGWSLRDLYRTLDMPGKNPLRDAQERLDAAVSAAYGLPAGADMLDFLLALNAEVAAAEARGAAVTGPGLPAGLNTADFVTADAVRPLG

SEQ ID NO: 23 - DNA, 2937 bp, Nitrobacter hamburgensis X14
gtgagcgaacgggtcgagcagatcgaggcatttgttgcctatgcgaaaacgttaaagggtgacgagaagggcgaagcacaggtgttctgtgatcgccttttccaagcttttggccacgaaggttataaggaagccggcgcggaactggagagtcgggtgaagaaggcgtccggaaagggcgtcaacttcgcagacttgatctggaaaccccgggttctgatcgaaatgaagaaaagcagcgaaaagctgcatcttcattaccagcaagccttcgattactggctgaacgcggtccctaaccgcccgcgatatgtggtgctctgcaatttcaaagagttctggatttacgactttgataagcaattaaacgagccagtagacgtcgtccggcttcaagacctgcccgcccggtacacggcgctaaactttctttttccagacaatccagacccgctgtttggcaacgatcgcgaagaggtctcgcgtgtagcggcctcaaaggtcgcgcagttatttcggtcgatggtcgctcgcggcattccgcgagagcaggcacaacgatttgtactgcaggccgtggtggcgatgtttgctgaagatatcgacatgatgccggccgggacgaccctgcggctagtgcaggactgcctggagcacggccaaaattcgtacgacgtgttcggtggcctgtttctccaaatgaacaataaggcggcggcgcagggcggccgctacaagggagttccttattttaacggcgggctatttgcgacggtccagccgatcgaattgactacggacgagctagagttgctcggcaagaaggatgaaggtgctgcttggcaaaactgggccaagatcaaccctgccatcttcggcaccattttccaacagagcatggacaagggggagcggcatgcgttcggcgcgcacttcacccatgaggccgacattcagcggattgtcgggcccacgattgtgcgtccctggcgcgaacgcatcgatgcagcgaagaccatggcggagctgctggagattcgcaaagcgcttctcaatttccgcgtcctcgatcccgcctgcggaagcggcaattttctgtacgtggcctacagagagatggtgcgtctcgaaatcaagctcatggccagactggacaaggagtttagctggaagaccgtacaaaagcaggctcaggccacatcgctcatcagccctcgccagttttttggtgtcgagcgggattcgttcggcgtcgagttgaccaaggtcaccctaatgctggcaaaaaagctggccctagacgaggccgccgatgttttggagcgcgaccagattgagttgccattggcggaggatgaggcgctcccactggacaacctcgatggcaacattctttgccgcgatgcgctcctatcggactggcccgaagtagacaccattatcggaaatcccccgtaccaaagcaaaaacaaggcacagcaagagttcgggcgtgcctatctgaacaagattcgatcggttttcccggagattgacggaagggccgattattgcgtctactggtttagaaaagcgcacgaccagctgaagcaaggccaaagagctggtctcgtcggcaccaatacgatccggcaaaactattcccgaatcagcgggctggattacatagccaagcacaacggtacgattacggaagcggtctctaccatgccgtggtcgggcgacgcggtcgtgcacgtttccatcgtcaactgggtgaaaggcgaggatgacggcaagaaacgcctgtacattcagtcaggcaatgatccggccggcggctgggattacaaggacctcgacgaaatcaacacctcgctttcgttttcaacggatgtgagccaggcgcaacgcatcaatgcgaacgctgaaaagggcggttgctatcagggccagacacacgggcataagggttttctcccggaaccggccgaagcgaaggcgatgatcaaggccagcaaggcaaacgctaaggtcctcttcccatttttgatcgccgacgatttcttgggtgcggtagacaaactcgaatgcagatacgtcatcgatttccaaacccgcgacctcctccaggccaaggcgttcaaaagaccgtttgagcatcttgaaaagacggtccttcctacccgaaaggaagctgcaaagaaggaaaaggatcgaaacaaggaagctttggacgccgacccggaagccaaggtcaacaagcaccacgaaaactttctaaagcgctggtggctgatgtcttacgcgcgcgaggacctgatgcagacgttggctcctttgagccgctacatcgtttgcgcacgcgttacgcacaggccaatctttgaattcgtctcgacagccattcatccgaatgacgcactgagcgttttcgccttggaggatgattactcctttggaatccttcaatcgggcatccattgggagtggtttatcaatcgatgctcgaccctcaaggctgactttcgctacacttcggatactgtctttgatagttttccgtggccccaggaacccagtgccgatgcggtgcgcctggtcgcgaagcgagctgtcgaggttaggcaacttcggtctaagctgaaggtcaaacatcacctgtcgctaagggagttgtatcgagcaatcgaaggtcctggagaacacgctctcaagaaagcccacaagcttctggacgaggccgtgcgcggagcttacggcatgtctaagaaggcggatgtattagaaacattactggaactgaacgagaccgtagtagctgcggaggccgacggaaaacaagtcgtcggccctggaatcccgccttcggcctcgaagctaaagaacctcgtcactactgataagctgacgatctcgccgacgagttgggccaataatgctcctgtaaaaacgtga

SEQ ID NO: 24 - protein, 978 aa, Nitrobacter hamburgensis X14
MSERVEQIEAFVAYAKTLKGDEKGEAQVFCDRLFQAFGHEGYKEAGAELESRVKKASGKGVNFADLIWKPRVLIEMKKSSEKLHLHYQQAFDYWLNAVPNRPRYVVLCNFKEFWIYDFDKQLNEPVDVVRLQDLPARYTALNFLFPDNPDPLFGNDREEVSRVAASKVAQLFRSMVARGIPREQAQRFVLQAVVAMFAEDIDMMPAGTTLRLVQDCLEHGQNSYDVFGGLFLQMNNKAAAQGGRYKGVPYFNGGLFATVQPIELTTDELELLGKKDEGAAWQNWAKINPAIFGTIFQQSMDKGERHAFGAHFTHEADIQRIVGPTIVRPWRERIDAAKTMAELLEIRKALLNFRVLDPACGSGNFLYVAYREMVRLEIKLMARLDKEFSWKTVQKQAQATSLISPRQFFGVERDSFGVELTKVTLMLAKKLALDEAADVLERDQIELPLAEDEALPLDNLDGNILCRDALLSDWPEVDTIIGNPPYQSKNKAQQEFGRAYLNKIRSVFPEIDGRADYCVYWFRKAHDQLKQGQRAGLVGTNTIRQNYSRISGLDYIAKHNGTITEAVSTMPWSGDAVVHVSIVNWVKGEDDGKKRLYIQSGNDPAGGWDYKDLDEINTSLSFSTDVSQAQRINANAEKGGCYQGQTHGHKGFLPEPAEAKAMIKASKANAKVLFPFLIADDFLGAVDKLECRYVIDFQTRDLLQAKAFKRPFEHLEKTVLPTRKEAAKKEKDRNKEALDADPEAKVNKHHENFLKRWWLMSYAREDLMQTLAPLSRYIVCARVTHRPIFEFVSTAIHPNDALSVFALEDDYSFGILQSGIHWEWFINRCSTLKADFRYTSDTVFDSFPWPQEPSADAVRLVAKRAVEVRQLRSKLKVKHHLSLRELYRAIEGPGEHALKKAHKLLDEAVRGAYGMSKKADVLETLLELNETVVAAEADGKQVVGPGIPPSASKLKNLVTTDKLTISPTSWANNAPVKT

SEQ ID NO: 25 - DNA, 3555 bp, Rhodopseudomonas palustris BisB5
atgggggactcaataagcgtaccggcagtcgagcagttcatcgcgcgttggcaaggccgtgaaggcggacaggaacgcgcgaactacgtctcgtttctcaccgagttgatcgcgctgctcgggctggacaagcccgacccggccgacgcgacgcatgagcacaacgactacgtgttcgaacgcgcggtgaagaagaccgccgaagacagcgcttcctatggccgcatcgatctctacaagcgcaacagcttcgtcctcgaagccaagcagagccggatcaagggcggcaagaaggaagtcaggggacagtacgatctgttgaagaccgaggccaccgcagcaacgctcggccgccgcggcgccgatcgcgcctgggacgtgctgatgctgaacgccaagcggcaggccgaggaatatgcccgcgccctgcccgcctcgcacggctggccgcccttcattctggtctgcgacgtcggccattgtatcgaggtctatgccgacttctccggccagggaaagaactacacgcagtttcccgatcgccagaacttccgcatctatctcgaggatctgcgcgaccacgacgtccgcgagcggctgcgcaagatctggagcgagccgaccgcgctcgacccgtcgcagcaatcggcgaaagtcacgcgcgacatcgccaagcggctcgcgcaagtgtcgctggcgctggagaaacagaactatccggccgacgacgtcgcgatgttcctgatgcgctgcctgttcacgatgttcgccgaggacgtcgaactgttgccggaaaaatccttcaagctgctgctcgaagactgcgagaaaaaccccgaggccttcgtccacgacgtcggtcagctctgggaggcgatggacaccgggcaatgggcgcacgcgctcaagaccaaggtcaagaaattcaacggcgagttcttcaagagccgcgccgcgctgccgctcggccgcgaggagatcggcgagctgcggcgggccgccgagtatgactggaacgaggtcgatccctcgatcttcggcacgctgctggaacaggcgctcgatccgaccgaccgcaagaagctcggcgcgcactacacgccgcgcgcttatgtcgaacggctggtgatcgccaccatcatcgagccgctgcgcgaggactggcgcaacgtccaggccaccgccgaaacgctgcgcggcgcaggcgatctcgctgccgccgccgccgcggtgcaggcgtatcacgaccggctgtgcgagacgcgggtgctcgacccggcctgcggcaccggcaacttcctttacgtctcgctcgaactgatgaagcggctggaaggcgaagtgctggaagctttgctcgacctcggcggccaggaagcgctgcgcggcctcggctcgcactcggtcgatccgcatcagttcctcggcctcgaaatcaatccgcgcgccgcggcgatcgccgagctggtgctgtggatcggctatctgcaatggcacttccgcaccaagggcgccccgcccgacgagccgatcctgcgcgccttcaagaacatcaaggtcaagaacgcggtgctcgactgggacggcgcgccgctgccgaagatcgtcgagggcaaagagacctatccgaacccgcgccggccggaatggccggcggcggaattcatcgtggggaatccgccgttcattggggcgagctttttgcgagcgcggcttggtgacacccacgctgaagcgctttggagtgcccatcctcaaatgaatgagtcggccgacttcgtgatgtactggtgggaccgcgcggccgaattgctgacccgcaaaggaacggtgctgcggcggttcggttttgtcacgacaaactcgataacccaagtatttcagcgtcgagtgatcgaaaggcacttcaaggcaaagaggccgatttcgcttgctatggcaattccagatcatccctggaccaaagctacaacggatgccgcagcggtacggatcgcaatgagcgttggagagactggccgaggcgatggactgctccagatcgtcgtcaacgaggctcacttggattcagatactccaatcgttgagcttcagggccgcgtaggaccgataaactcagacctcacaattggcacagacctgaccaccaccgtgcctctacgtgcatctgaaggcttggcatctcgtggagttacgcttgcaggctctggattcttgataacttcagaagaagccgaacattttggtctcggtacgcacgagaagctaaagcaacatattcgaggactccataatggacgcgacctgaatcagacatcacgtcgaattcttgtgctcgacttcttagggctgagcgaagaggaagtccgaaggcattttccagaagcatatcagcatctactccggacagtgaaacccgaacgggaaacgaacaagagagcatcctataggcagaattggtgggtgtttgctgagccgcggaaggagatgcgtcccgcgctgaaggacttggggcgctatatcggtacggcacgcaccgctaagcataggattttctccatgttggcgggccactccttaccagagagtgaggttattgcggtggggtcagacgacgcgtttatattgggagtactttcgtcacgacttcatgttcgctggagtctgtccaaaggtggcacgctggaagacaggcctcggtacaataacagcatgtgcttcgatcccttccccttccccgacgccaatccgattcagaagcagaccattcgggtcatcgccgaggagctcgacgcgcatcgcaagcgggtgctggcggagcatccgcatctgacgctgaccgggctgtataatgtgctggagcggttgcgggcgggggctgtgccgcaggcacagccgtcacccgcgggcttgacccgcgggtccacgtcgtcacgcggtgcggcgaagaaagacctggatggccggggcactggacggcaagacggcgcttcgcgcctttcgcccggccatgacgatgcagagatggtgctcacacccgacgagcagtgcatcttcgacgatggcctggtgctgatcctgaaagaactgcacgacaggctcgatgtcgcggtggccgaggcctatggctggccggcgaacctgtccgacgacgagattttggcgcggctcgtcgctttgaacaagcagcgcgccgacgaggaaaagcgcgggctggtgcgctggctgcggcccgactaccagattccgcgattcgccaagggcgtcgacaagcaggcggcgaaggaagaaggcgcgcagatcgcagcgtcgctcgatctcggcgagacccggcagaagccgtcgttcccgaccggtgcggtggagcagaccgccgcggtgttcgcagcgctggccgcagcctccggcccgctcgacgccaaatcgctcgccgcgcagttcaggcgcacgaagacgaccgagaagaaactcgccgaggtgctcgcctcactggcgcggctcggctacgtggcgaccaccgacggcgtcagcttcgcgctgcgccgggtcgcgtag

SEQ ID NO: 26 - protein, 1184 aa, Rhodopseudomonas palustris BisB5
MGDSISVPAVEQFIARWQGREGGQERANYVSFLTELIALLGLDKPDPADATHEHNDYVFERAVKKTAEDSASYGRIDLYKRNSFVLEAKQSRIKGGKKEVRGQYDLLKTEATAATLGRRGADRAWDVLMLNAKRQAEEYARALPASHGWPPFILVCDVGHCIEVYADFSGQGKNYTQFPDRQNFRIYLEDLRDHDVRERLRKIWSEPTALDPSQQSAKVTRDIAKRLAQVSLALEKQNYPADDVAMFLMRCLFTMFAEDVELLPEKSFKLLLEDCEKNPEAFVHDVGQLWEAMDTGQWAHALKTKVKKFNGEFFKSRAALPLGREEIGELRRAAEYDWNEVDPSIFGTLLEQALDPTDRKKLGAHYTPRAYVERLVIATIIEPLREDWRNVQATAETLRGAGDLAAAAAAVQAYHDRLCETRVLDPACGTGNFLYVSLELMKRLEGEVLEALLDLGGQEALRGLGSHSVDPHQFLGLEINPRAAAIAELVLWIGYLQWHFRTKGAPPDEPILRAFKNIKVKNAVLDWDGAPLPKIVEGKETYPNPRRPEWPAAEFIVGNPPFIGASFLRARLGDTHAEALWSAHPQMNESADFVMYWWDRAAELLTRKGTVLRRFGFVTTNSITQVFQRRVIERHFKAKRPISLAMAIPDHPWTKATTDAAAVRIAMSVGETGRGDGLLQIVVNEAHLDSDTPIVELQGRVGPINSDLTIGTDLTTTVPLRASEGLASRGVTLAGSGFLITSEEAEHFGLGTHEKLKQHIRGLHNGRDLNQTSRRILVLDFLGLSEEEVRRHFPEAYQHLLRTVKPERETNKRASYRQNWWVFAEPRKEMRPALKDLGRYIGTARTAKHRIFSMLAGHSLPESEVIAVGSDDAFILGVLSSRLHVRWSLSKGGTLEDRPRYNNSMCFDPFPFPDANPIQKQTIRVIAEELDAHRKRVLAEHPHLTLTGLYNVLERLRAGAVPQAQPSPAGLTRGSTSSRGAAKKDLDGRGTGRQDGASRLSPGHDDAEMVLTPDEQCIFDDGLVLILKELHDRLDVAVAEAYGWPANLSDDEILARLVALNKQRADEEKRGLVRWLRPDYQIPRFAKGVDKQAAKEEGAQIAASLDLGETRQKPSFPTGAVEQTAAVFAALAAASGPLDAKSLAAQFRRTKTTEKKLAEVLASLARLGYVATTDGVSFALRRVA

SEQ ID NO: 27 - DNA, 27 bp, artificial
  primer
gattatagatattctgccagcctggtt

SEQ ID NO: 28 - DNA, 28 bp, artificial
  primer
actttctaaccttcctcctacatttctc

SEQ ID NO: 29 - DNA, 26 bp, artificial
  primer
cgctatcgctactctaataccgtcgt

SEQ ID NO: 30 - DNA, 21 bp, artificial
  primer
gcttttcagacgacctgcaac

SEQ ID NO: 31 - DNA, 44 bp, artificial
  primer
actttttaaccttcctgctacagttctcatccagcagttgtgca

SEQ ID NO: 32 - DNA, 42 bp, artificial
  primer
gctttccagacgacctccaacgttacgcataaaggcgttgtg

SEQ ID NO: 33 - DNA, 3483 bp, Pseudomonas species OM2164
ctggaaatcggcttgagtgtcccgaaacaggcaggaccgatcttgagcgtcgatgatttcatcgcccgctggacgacctcgggtggcagcgagcgggccaatttccagcagttcgccatcgagctgacgcagctcttggacgttccggcccccaagcccgcgacggcggatgcgcagaacgacgactaccgcttcgagcggcccgtgaccttcattcataccggcacgcagtcgcgcggcttcatcgacctctaccggcgcggctgcttcgtcatggaagccaagcagggcacaggcgccgcgcccgaggaaggccagcttgatcttctagccgcggccccgcccgtgcagcggcaagggcatggcgttcgcggctcgaagcgatgggacgacaccatgctgcgcgcccgcaaccaggccgacggctatgcccgcgccgtggcgcgcgaggacggctggcccccgttcctgctgatcgtggacgtgggccatgtgatcgaggtctatgccgacttctcgggccaggggcagggctacacgcagttcccggacggcaaccgctaccggatcacgctggacgacctgcgcgacgcggcgacccttgaccgcctgcaagccatctggaccgatccgcacagcctcgacccgacccgcgtcagcgcccaggtcacgcggcaggtggccgagcatctggccgaactgggtcggtccttcgaggcgcagggccatgcccccgaggcggtggcgcgcttcctgatgcgcgccctgttcaccatgttcgccgaggacgtgcaactgatccccgagggggccttttcgaagctgctgcaggacaggcgcggccaccccgaacacgccgccccgatgctggaaagcctgtggcagacgatgaacaccggcggcttttccccggcgctgtcctgcgacctcaaacggttcaacggcggcctgtttcgggaggcaaccgccctgccgctgtccgccatgcagcttggcctgctgatccaggccgcgtcccacgactggcgcgaggtcgagccggcgatcttcggcaccctgctggaacgcgcgctcgacacgcggcagcgccacaagctgggcgcgcactacaccccccgcgcctatgtcgaacggctggtgaaccccacggtgatcgagccgctgcgggccgaatggcgcgacatccaggccgcggccgtcacgctggcaggccaggacaagctggacgaggcgcgcgcgaccgtgcgcgacttccaccggcgcctgtgcgaggtgcgggtggtggacccggcctgcgggtcgggaaacttcctgtatgtcgcgctggagctgatgaagcgcctggaaggcgaggtgatcgcgctgctgcgcgagttgggcgaggaccagggcgcccttgccctggcaggccacaccgttgacccgcaccagttcctgggcatcgaggtgaacccctgggccgccgccgtggccgagctggtgctgtggatcggctatctgcaatggcatttccgcacccatggcaccgccagcccggccgagccggtcctgcgcgacttccgcaacatcgagaaccgcgacgccgtgctggcctgggacggcacccggccgaggctggacgatgccgggcagcccgtgacccgctgggacggggtgtccaccatccgccacccggtcacgggcgaacaggtgcccgatccggccgcgcgggtgcaggttctggattacctcaagccgcgcccggccagatggcccgaggccgagttcatcgtcggcaacccgcccttcatcggcgcgtcgcggatgcgcgaggccctgggcgacggctatgccgaggccttgcgcgcggcctatcccaggatgcccgaaagcgccgatttcgtgatgttctggtgggataaggcggcgctggcgacccgcgcgggcaagacccggcgctttggcttcatcaccaccaattcgctgcgccagaccttcaaccggcaggtgctggaaccgcatctggccgacccgaagaagcccttgtcgctggccttcgccatccccgatcacccctgggtcgatgcgggggacggcgcggcggtgcggatcgccatgaccgtggcagcggccggatcggcgccggggcggctgtttaccgtcacggacgaacgccggggcgagcgcgaggccgaggggcgccccgtcaccctgtccgggcagatcggcaagatccacgccaacctgcggattggcgcggatgtggcgggagcgaaaccgctgcgggcgaacgcaggcatctcatcgccgggggtgaagctgcacggcgcaggcttcatcgtcaccccggccgaggcacaggcgcttggcttgggcaccgtgccgggtcttgaggcgcatatccgcagctatcgcaacggccgcgacctgaccgccaccccgcgtggcgtcatggtgatcgacctgttcggcctgtccgaggccgaggtgcggacccggtttcccgccgtttatcagcacgtcctggacaaggtgaaacccgagcgcgaccagaacaaccgcgacagctacaagcgcaactggtggattcacggcgagccgcgccgcgacctgcgcccggccttggaaggcttgccccgctacatcgccacggtggaaacggccaaacatagaatattcagcttactcgacgcgacgattttacccgacaacaagttgatcatcatcgctctggcagacacatggcatttttcgattgtgtcatcgcgtatccactgggtctgggcgatagcaaatgctgcgaaaatcggcatgtatgatggcgatgccgtttaccccaagggtcaatgcttcgaccccttccctttcccagatgccaccgaggcacagaaagcccgcctgcgcgccttgggcgaggaactggacgcgcatcgcaaggcgcagcaggccgcgcatccccggctgaccctgacggccctctacaacgtgctggaaaagctgcgcgccggcgagcggatcgaggggcgcgaccgggaaacctatgacgcgggcctcgtcggcatcctgcgggacatccacgaccgcatcgacgccgccgtggccgaggcctatggctggcctgccgacctggacgacgaggccatcctgacccgcctggtcgatctgaaccgcgcccgcgccgccgaggaagcggcgggcctggtccgctggctgcgccccgactatcagaaccccgcaggccgcattgccgccgccaagggccagcaggtcgaactggacgtgggcgcggcggccgaggccgccgacaaggcgctgtggcccaaggccctgcccgaacagatcgccgccgtccgcgccgtcctgtcggacatgggcgaggccacgcccgaacaggtcgcgcgccagttcaaacgcgcccgcgcggcgtcggtgaagcccctgctggaaagcctcagcgccttgggtcaagcccgcctcatcgaaggcgggcggttcgcggcctga

SEQ ID NO: 34 - protein, 1160 aa, Pseudomonas species OM2164
MEIGLSVPKQAGPILSVDDFIARWTTSGGSERANFQQFAIELTQLLDVPAPKPATADAQNDDYRFERPVTFIHTGTQSRGFIDLYRRGCFVMEAKQGTGAAPEEGQLDLLAAAPPVQRQGHGVRGSKRWDDTMLRARNQADGYARAVAREDGWPPFLLIVDVGHVIEVYADFSGQGQGYTQFPDGNRYRITLDDLRDAATLDRLQAIWTDPHSLDPTRVSAQVTRQVAEHLAELGRSFEAQGHAPEAVARFLMRALFTMFAEDVQLIPEGAFSKLLQDRRGHPEHAAPMLESLWQTMNTGGFSPALSCDLKRFNGGLFREATALPLSAMQLGLLIQAASHDWREVEPAIFGTLLERALDTRQRHKLGAHYTPRAYVERLVNPTVIEPLRAEWRDIQAAAVTLAGQDKLDEARATVRDFHRRLCEVRVVDPACGSGNFLYVALELMKRLEGEVIALLRELGEDQGALALAGHTVDPHQFLGIEVNPWAAAVAELVLWIGYLQWHFRTHGTASPAEPVLRDFRNIENRDAVLAWDGTRPRLDDAGQPVTRWDGVSTIRHPVTGEQVPDPAARVQVLDYLKPRPARWPEAEFIVGNPPFIGASRMREALGDGYAEALRAAYPRMPESADFVMFWWDKAALATRAGKTRRFGFITTNSLRQTFNRQVLEPHLADPKKPLSLAFAIPDHPWVDAGDGAAVRIAMTVAAAGSAPGRLFTVTDERRGEREAEGRPVTLSGQIGKIHANLRIGADVAGAKPLRANAGISSPGVKLHGAGFIVTPAEAQALGLGTVPGLEAHIRSYRNGRDLTATPRGVMVIDLFGLSEAEVRTRFPAVYQHVLDKVKPERDQNNRDSYKRNWWIHGEPRRDLRPALEGLPRYIATVETAKHRIFSLLDATILPDNKLIIIALADTWHFSIVSSRIHWVWAIANAAKIGMYDGDAVYPKGQCFDPFPFPDATEAQKARLRALGEELDAHRKAQQAAHPRLTLTALYNVLEKLRAGERIEGRDRETYDAGLVGILRDIHDRIDAAVAEAYGWPADLDDEAILTRLVDLNRARAAEEAAGLVRWLRPDYQNPAGRIAAAKGQQVELDVGAAAEAADKALWPKALPEQIAAVRAVLSDMGEATPEQVARQFKRARAASVKPLLESLSALGQARLIEGGRFAA

SEQ ID NO: 35 - DNA, 3435 bp, Deinococcus radiodurans
atgacgcctgaggaatttataacccgctggtcgccctccggaggcgcggaacgcgccaattacgtcctctttctcagtgagctgtgcgatctgctcggcgtgcccaagcccgaccccacccaggccgatgaagctaagaacgcttacgtcttcgagaaggacgttcccgacctgcacgatgacggcggcctcagccagcgccgcatcgacctctaccggcggggcgcgttcatcttggaggccaagcagggggtcgagaaggaagctaccgctgaagaagctctcctcagcaccaagggcaagaagaaaaagggacatggcacgcggggcaccaaaggctgggacaccttcatgcgccgcgccagggagcaagcggagcgctacgcgcacctgctgcccgcatccgagggccggccccccttcctgctcgtggtggatgtcgggcatgtcatcgaggtctacgctgagttcacgcgtaccggtggggcgtatctccccttccccagtgccagagcgcaccagatccaattggctgacctggcccgacctgaagtccgtgagctgctgcgcaccatctggctcgatcccctgagtctcgaccccagcatccacgcggctgaggtcaccaaggacgtggcccgcaagctcgcggagatcagccgcagcatggaagggcagcccgatgcccagggacaggcgatgacgccagagcgcgtttcgcagttcctgatgcgcatgatcttcaccatgttcgccgaggacgtcggcctgctgcccaacaccaagttccgcgacaagctcaagtccttgctcggacggccccaggccttcattcccaccatcaccgatctgtggcaggcaatggcgaagggcggatacagcgtggccctcgatgcacagatcaagcatttcaacggcggtctgttcgagggcgtggaagtcctgcctgtgaccgatgggcagctcaagctctttatcgaagctgccgagtccgactggagccgcgtcgaacccagcatcttcggcacgctcgtcgagcgtgccctgaacccccgcgagcgccaccgcctgggagcccactacaccccccgtgcctatgtcgagcgcctggtgcatcaggtggtgatggagcctctgcgcgaggactggcgcaccgtgcaggttcaggtgcaggacaccctcgaccggggcaacggggacgacaaggcccgggccagggcacagcaactcgtcgcgcagttccatgcccagctgcggcagacccaggtgctcgatcctgcctgtgggacggggaacttcatctacgtcagcatggaactgatcaagcggctggaggcggaggtcattgaaacgctggtggccctgggcggcctgccgcccctgatcgaggtgaaccccgagcagtttcacggcatcgaggtcaacccacgtgccgcgagcgtggccgagctggtgctgtggatcggctacctgcagctctacgcccgtgagcacggcaacgccgcgccgcccgagccgatcctgcgggccttccacaacatcgagaaccgcgacgccgtgctgagttacagccatacgacgccgaaagtagatagggacggccagcccgtgacccgctgggacggggtgacattcaggcgtcacccagtgaccggagatcctgtgcccgacgaaagggcacagataccggaagaggtctaccacaatccaatgactaccgagtggcccaaggcggactttattgtcggcaatcctccgttcattggtagtaaacgcatgcgggaactgctgggcaatggttatgtggacgctttacaaagggtatttgctgacgtgccacaggccaccgattttgttcttcgttggtggtataaagctgcgttactgaccaggcaggaggaagttaggcgattcggtttcatcacgactaacagcattagccaagcgtttaatcgccgtgctatcgaacctcacttaaacgctgacgttagacctctttcactcgtgtacgtcacaccagaccatccgtgggtagatgaatccgacggtgcagccgtacgtattgcgagtacggttggggagctcggacaacgccctggcttacttgcgcgtgtggtcaaagaatatgatgaagctgcagagggcgatctggtagctgaatttgcctttgaaacaggtgtaattcatgctgacttgagcataggggcggacttaacggagactcagccactcatggcaaatctcggtctttgtgccgtaggcatgaagactataggggccggttttctcgtggagcgtacgaaagccgaggctctgggccttggtcaggataatcggattcgtccctatatcaacgggcgcgatctaatgggtcgtactcgcggtgtgtatgtaatcgatctcttcggtgtctcggaagaagatgtgcgcgatcaatatccaaaactctatcaacatttgagaaatgctgtgtacgacatacgtcgccagaacaacaatagggtttttcgtgatttatggtgggttattggccatccacgtccaatcttccgtgaatttacgcggggcttgaaaagatatgtggttactttagaaactgccaagcaccaagtattccaattccttgacagctctatcgttccagacagtaccatcgtcacctttggaactgaggatgcatttcaccttggcgtcctgagcagccgtgtccatgtcacctgggcgctcgcgcaagggggcaccctggaggacaggccccgctacaacaagacccggtgcttcgaaaccttccccttcccggcggccacgcctgagcagcagcaacgcatccgtgacctcgccgagcgcctggacgcccaccgcaaggcgagactggccgagcatcccaagctgaccatgacggatatgtacaacgccctggccgcccttcgtgccgggcaacccctggagggcaagctcaagacggcccacgaccagggcctggtgaccaccctcaggcagctgcatgacgacctcgacgtggcagtcctggctgcctacggctggcctacaggactcgatgagcaaggcctgctggaaaggctcgctgccctgaacgccgagcgggtacaggaggaaaaggcaggccgcattcgctatctccggccggcctaccaggatccgcacggcaccgcgcaggagaacctagggatggccgtggccagccgcccggcgaaggctgctcaggtcatgccctttcccacggccctgccccttcaggtgcaggccgtcagaagtgcccttatgcaggcggggcaggccctcagcccccaggaggtcgcccaggccttccaaggggccaaagaaaagcaggtcgaggacatcatgcagaccctggtgctgctggggcaggcccacctccgcgagcacaatggggaggtgaggtatgccgcctga

SEQ ID NO: 36 - protein, 1144 aa, Deinococcus radiodurans
MTPEEFITRWSPSGGAERANYVLFLSELCDLLGVPKPDPTQADEAKNAYVFEKDVPDLHDDGGLSQRRIDLYRRGAFILEAKQGVEKEATAEEALLSTKGKKKKGHGTRGTKGWDTFMRRAREQAERYAHLLPASEGRPPFLLVVDVGHVIEVYAEFTRTGGAYLPFPSARAHQIQLADLARPEVRELLRTIWLDPLSLDPSIHAAEVTKDVARKLAEISRSMEGQPDAQGQAMTPERVSQFLMRMIFTMFAEDVGLLPNTKFRDKLKSLLGRPQAFIPTITDLWQAMAKGGYSVALDAQIKHFNGGLFEGVEVLPVTDGQLKLFIEAAESDWSRVEPSIFGTLVERALNPRERHRLGAHYTPRAYVERLVHQVVMEPLREDWRTVQVQVQDTLDRGNGDDKARARAQQLVAQFHAQLRQTQVLDPACGTGNFIYVSMELIKRLEAEVIETLVALGGLPPLIEVNPEQFHGIEVNPRAASVAELVLWIGYLQLYAREHGNAAPPEPILRAFHNIENRDAVLSYSHTTPKVDRDGQPVTRWDGVTFRRHPVTGDPVPDERAQIPEEVYHNPMTTEWPKADFIVGNPPFIGSKRMRELLGNGYVDALQRVFADVPQATDFVLRWWYKAALLTRQEEVRRFGFITTNSISQAFNRRAIEPHLNADVRPLSLVYVTPDHPWVDESDGAAVRIASTVGELGQRPGLLARVVKEYDEAAEGDLVAEFAFETGVIHADLSIGADLTETQPLMANLGLCAVGMKTIGAGFLVERTKAEALGLGQDNRIRPYINGRDLMGRTRGVYVIDLFGVSEEDVRDQYPKLYQHLRNAVYDIRRQNNNRVFRDLWWVIGHPRPIFREFTRGLKRYVVTLETAKHQVFQFLDSSIVPDSTIVTFGTEDAFHLGVLSSRVHVTWALAQGGTLEDRPRYNKTRCFETFPFPAATPEQQQRIRDLAERLDAHRKARLAEHPKLTMTDMYNALAALRAGQPLEGKLKTAHDQGLVTTLRQLHDDLDVAVLAAYGWPTGLDEQGLLERLAALNAERVQEEKAGRIRYLRPAYQDPHGTAQENLGMAVASRPAKAAQVMPFPTALPLQVQAVRSALMQAGQALSPQEVAQAFQGAKEKQVEDIMQTLVLLGQAHLREHNGEVRYAA

SEQ ID NO: 37 - DNA, 3456 bp, Marinobacter aquaeolei VT8
ttggaagccttcattgcagcctccgctgctgtcgacgaattcctcaaacgctggaaaggcaacacaggtagtgaacgcgcaaactttcaatcgttcatgcgagacctgtgtacgctgctggaccttcctcatccagacccaggtgaaggtgacaccactcagaacgcctatgtatttgagcggtttatcgcgtcggctcgagtcgatggcaataccgacaaccggtacatcgacctgtatcgtcgggactgcttcgtactggaagggaagcagactggcaaggagctggcatcccgaagccaacagaacgctgttaatgcagctgtagcacaggctgagcgatacattcgaggactgccccaggaagaagtagagcatggccgcccgccattcatcgtgatcgtcgatgtgggcaacgccatctacacgtactccgagttctcgcgaactggcggtaactatgttccattccctgatcccagacactatgagatccgactggaagacctgcacaaaccagatgttcagcaccgtcttcgtcagttatggctagaaccggatcagctcgatccgagtaagcatgctgccagggtgacccgagaggtcagcaccaagctggctgaattggcaaagtccctggagcataatggatacgatgtcgagcgagtagccagctttctcaagcgctgcctgttcacgatgtttgccgaagacgtagagttgctgcccaaggcatccttccagaaccttttgatcgacattaaggaccggaaccctgaagccttcccccacgccgtgaaggcgctttgggaaaccatgaatgctggtggctacagtgagcgtctgatgcagaccatcaagcgatttaacggtgggttgttcaaaggcatcgatccaatcccgctgaatgttcagcagatccaacttctcatagatgcggccaaagccgactggcgtttcgttgaacctgccatcttcgggacgctgctagagcgtgcccttgatcctcgggagcgccacaagctgggcgcccattacactcccagggcctacgttgaacgcttggtcatgccgaccctgattgaaccgcttcgtgagcaatggggcgacatccgaggtgcggcggaaaccctgctgcggcaaggcaaaacagacaaagctcttcaggaagtccaagccttccattatcagctttgccagacccgagtacttgatcccgcttgtggtagcgctaacttcctttacgtggcccttgaacacatgaagcgcctggagggggaggtcctgggttttatctccgagctgacccaggggcaaggcgtgctggaaagtgaaggcctgaccgtcgatccgcaccagttcctgggcttggagataaacccacgagcagcccagattgccgaactcgttttgtggattggctaccttcagtggcactaccggctgaacgaccggctggacctccccgagcccatcttgcgggacttcaaaaacattgagtgcagggatgctctgatcgagtatgacagtcgagaaccggagctaaataaaaatggggaaccggtgaccatctgggatggcatcagcatgaaggtgagcccgacaacgggtgaattaatccccgatgaaacagggcgagctaaggtctaccgttaccacaatccacgcagggctgagtggccagcagcagagtacataataggaaatcctccttatattggcgctcgccgaattagatccgccttgggtgacggttatttacaagcgttgcgaggcgtatacaccgatattccagaacacgtcgatttcgtcatgtattggtgggcaaaggcttcagagaacatggcaagtggtaaaacaaaagcgtttggattaattaccacgaatagtcttcggcaaagcttttctcgaaaggttgtagaaaaaaccttagatatcaattcggactgttccataaaattcgtgattcctgatcatccgtgggttgatagcgccgacggtgcggcggttcgggtcacattgatttctgttgacagcaataaagcgcccggaatagttgctctcatcagaaacgaggaagcagaaggtagtggagcctacaagattaccttggataacaagtcggggcatataacgccgaacctcacgataggggcggaccccggagaagctacgtgcttatcatcaaattcctcagtgtcatgcgtaggttatcaactaaccggcaaagggtttgttcttactcaaagccaaaaagaagagcacgaaaatgaatggcccgaaagtgtcattaaacctttgtggagcgggcgtgacatcacgcagtcacccagaaaaaactgggcaattgatgtttgtgattggggaattgacgctttaaaagtttcatcaccaagtctctatcaatggcttctcactcgggtaaagccggagcgcgaacagaacaatagagccagtctaaaggagcgttggtggatttacggcgaagccagaaacactttccggcccgctcttattggcatagaaacagctatcgcaacttctttaactgcgaaacatcgggtgtttgtgcacctagattcaaacagcatttgcgatagcaccactgtcatgttcgcactaccaggagcccagtaccttggtgttttaagttccagggtgcatgtactttggtcactttttgctggggggacactcgagaatcgtccgaggtataacaagacactgtgctttgaaacatttccttttccaaaaatgagttctgatcagtctgaaaaaataagtgacctcgcagaaaaaatagatcaagtacgcaaaggccaacaggcaaaacaccccgatctaacactaacggggatgtacaacgtgctcgaaaaactacgttccggtgaagagctaaccaacaaagaaaagaccatccacgaacaaggcttggtgtccgtactccgtgagctccacgacgacctcgatcgtgccgttttccaggcctatggttggtcagacttggcagataagcttgtaggtcgcccaggcgccacaaccccacttccagacaaaccggctgaacaagcggaggctgaggacgagctgttgatgcgattgctcgaactcaacaagcagcgtgcagaggaagaatcacggggcatagttcgctggttacgtccggattaccaggcgcgcgatgctgtacagacagaagtggatatcgcgccgaaggccgccgccacaaaaacggaagcctctaccagcaaaggaaaagcctcattcccgaaagcgattcccgatcagcttcgagtgctccgagaggcactcgcagagcgatctcacacgacggaaagtttggctgagatgttcaagcggaaacctatgaaatcggtcgaggagggtttgcagtcacttgtagctgtgggtgttgccgaatacgacccggaaactcaaacatggcatacggtatga

SEQ ID NO: 38 - protein, 1151 aa, Marinobacter aquaeolei VT8
MEAFIAASAAVDEFLKRWKGNTGSERANFQSFMRDLCTLLDLPHPDPGEGDTTQNAYVFERFIASARVDGNTDNRYIDLYRRDCFVLEGKQTGKELASRSQQNAVNAAVAQAERYIRGLPQEEVEHGRPPFIVIVDVGNAIYTYSEFSRTGGNYVPFPDPRHYEIRLEDLHKPDVQHRLRQLWLEPDQLDPSKHAARVTREVSTKLAELAKSLEHNGYDVERVASFLKRCLFTMFAEDVELLPKASFQNLLIDIKDRNPEAFPHAVKALWETMNAGGYSERLMQTIKRFNGGLFKGIDPIPLNVQQIQLLIDAAKADWRFVEPAIFGTLLERALDPRERHKLGAHYTPRAYVERLVMPTLIEPLREQWGDIRGAAETLLRQGKTDKALQEVQAFHYQLCQTRVLDPACGSANFLYVALEHMKRLEGEVLGFISELTQGQGVLESEGLTVDPHQFLGLEINPRAAQIAELVLWIGYLQWHYRLNDRLDLPEPILRDFKNIECRDALIEYDSREPELNKNGEPVTIWDGISMKVSPTTGELIPDETGRAKVYRYHNPRRAEWPAAEYIIGNPPYIGARRIRSALGDGYLQALRGVYTDIPEHVDFVMYWWAKASENMASGKTKAFGLITTNSLRQSFSRKVVEKTLDINSDCSIKFVIPDHPWVDSADGAAVRVTLISVDSNKAPGIVALIRNEEAEGSGAYKITLDNKSGHITPNLTIGADPGEATCLSSNSSVSCVGYQLTGKGFVLTQSQKEEHENEWPESVIKPLWSGRDITQSPRKNWAIDVCDWGIDALKVSSPSLYQWLLTRVKPEREQNNRASLKERWWIYGEARNTFRPALIGIETAIATSLTAKHRVFVHLDSNSICDSTTVMFALPGAQYLGVLSSRVHVLWSLFAGGTLENRPRYNKTLCFETFPFPKMSSDQSEKISDLAEKIDQVRKGQQAKHPDLTLTGMYNVLEKLRSGEELTNKEKTIHEQGLVSVLRELHDDLDRAVFQAYGWSDLADKLVGRPGATTPLPDKPAEQAEAEDELLMRLLELNKQRAEEESRGIVRWLRPDYQARDAVQTEVDIAPKAAATKTEASTSKGKASFPKAIPDQLRVLREALAERSHTTESLAEMFKRKPMKSVEEGLQSLVAVGVAEYDPETQTWHTV

SEQ ID NO: 39 - DNA, 2787 bp, Parvibaculum lavamentivorans DS-1
atgcggctgagctggaacgagattcgcgcccgcgcagcgcgtttttccgaggaatggaaaggtgtcacgcgcgaacgcgccgagacgcagaccttctataatgagttcttccagattttcgacatcccgcgccgtcgcgtcgcctcttacgaagagccggtaaagggccttggcgacaagcgcggctatatcgaccttttctggaaaggcacgcttcttgtcgagcacaagaccacgggccgcgacctcaaaaaggcaaagattcaggcgctcgattatttcccgggcctgaaggacaaggaactcccacgctacctcctcctctgcgatttccagagcttcgagctttacgatctggacgaagacaccgaggtccgtttccgcctcgccgatctgaaagatcatgtggaagccttcggcttcatgatcggcgtccagaagcgcaccttcaaggatcaggaccccgtcaacatcgaagcctcggagctgatgggcaagctccacgatgcactgaaggaatcgggttacgacggccacgaccttgagcaatatctggtccggcttctcttctgcctctttgccgacgacaccggcattttcgagcccaaggacatccttctcgatttcatccagaaccgcacaagcgcggatggcagcgatctcggctcccgcctcaatgaattgttcgaggtgttgaacacgccggaagacaagcgccagaaaacccttgatgaagacctcggaaatttcccttatgtgaatggcgcgcttttcgccgagcgtctgcgcacgcctgccttcaacgccgccatgcggctgatccttatcgaagcctgcgagttcaaatgggaggcaatctcgcctgccattttcggtgctctgttccagtccgtcatgaacaagacagagcgccgcgccctcggcgcgcattacacgaccgagaaaaacatcctgaaactcattcagccgcttttcctcgacggcctgcatgaagagttcgcgcgcgcaaaggcgctgaagcgcggccgccagcaggcgctggaagccttgcacgagaaactcggccagctcaccttcttcgatcccgcctgcggctgcggtaacttcctcgtcatcgcctatcgcgagctacgcgcgctggaacaggaaattctgcgcgtcctgcacgacggcaaagaccagcgcattttcgacgtggcgcaattgtcgaaagtcaatgtcgatcagttttacggcatcgaaataggcgagtttcccgcccgcatagccgaagtcgcgatgtggatgatggaccacatcatgaataacaggctcggcctctccttcggctccaactatgcgcgcatcccccttcggacctcaccgcacatcctccatgccgacgcgctggaagccgattgggccgctctcctcccgccggaaaaatgctcctatgtcttcggcaatccgcctttcatcggctcaaaattccagacggcggaacagcgtcggcaagtgcgtgacatcgcaaagctcggcggctccggcggcacgcttgatttcgtcaccgcatggttcctgaaggccggcgaatatgtgcagcatggaaaagcggacatcgccttcgtcgccaccaactcaatcacgcagggcgaacaggtcgcccagctctggccgctcctctttcagcgctgcaagctcgaaatcgccttcgcccaccgtaccttcgcctggggctcggacgcgcgcggcgtcgcccatgttcatgtcgtcatcatcggcctcacaaggcgcgaccgcgaatggcccgagaagcgcctcttctcttacgccgacatcaagggcgatccggtcgagacacgccacaaggctctgacggcttatctttttgatgccgtcaatgtagctgacagacatctagtagtcgaagaacgaaacactcctttgtgcgaagcgccgaaactcaaaactggcgttcagatgatcgacaacggcatcctcactttcacgacaatggaaaaggaggaatttcttcgtcaggagccggaagcggaaccgctgttccgcaaatacatcggtggcgatgagtatataaatggatttttccgatggatactctatctcgcagatgccgagccgagttttcttcgacagcttccgcttgttcaagaaagaatacggcaggtacgtcaataccggttatcgagttctcggcccagcacggtgagaatggcggactatccaacgcaggttggtgtggacgagcgattgagcggaccctatttggtgatacccaatacaagctcggagcgacgcgactacgtaccgatcggctggctgactcccgaggtagtagccaatcagaaattgcgcattcttcctgacgcagatccgtggatattcggtttgctgacaagcggcatgcacatggcttggatgcgcgcaatcaccggtcgcatgaaaagcgactacatgtattctgtcggcgtcgtctacaacactttcccttggccggatattaccgaagctcagaaacagaaaatccgtgcgctagcgcaagctgtgctcgacgcccgcgcgctttatcccggtgcaacgctggccgatctctacgatcccgacctgatgaaacgcgaactccgtcaggctcaccgagccctcgatgccgccgtcgacaaactctatcgcggccaagccttcgcaaatgaccgcgagcgtgtcgaacacctcttcggcctatacgaaaaactctcctccccgctgacagcagcaccgaagcccattaagcggaaacgaaagaaagagtag

SEQ ID NO: 40 - protein, 928 aa, Parvibaculum lavamentivorans DS-1
MRLSWNEIRARAARFSEEWKGVTRERAETQTFYNEFFQIFDIPRRRVASYEEPVKGLGDKRGYIDLFWKGTLLVEHKTTGRDLKKAKIQALDYFPGLKDKELPRYLLLCDFQSFELYDLDEDTEVRFRLADLKDHVEAFGFMIGVQKRTFKDQDPVNIEASELMGKLHDALKESGYDGHDLEQYLVRLLFCLFADDTGIFEPKDILLDFIQNRTSADGSDLGSRLNELFEVLNTPEDKRQKTLDEDLGNFPYVNGALFAERLRTPAFNAAMRLILIEACEFKWEAISPAIFGALFQSVMNKTERRALGAHYTTEKNILKLIQPLFLDGLHEEFARAKALKRGRQQALEALHEKLGQLTFFDPACGCGNFLVIAYRELRALEQEILRVLHDGKDQRIFDVAQLSKVNVDQFYGIEIGEFPARIAEVAMWMMDHIMNNRLGLSFGSNYARIPLRTSPHILHADALEADWAALLPPEKCSYVFGNPPFIGSKFQTAEQRRQVRDIAKLGGSGGTLDFVTAWFLKAGEYVQHGKADIAFVATNSITQGEQVAQLWPLLFQRCKLEIAFAHRTFAWGSDARGVAHVHVVIIGLTRRDREWPEKRLFSYADIKGDPVETRHKALTAYLFDAVNVADRHLVVEERNTPLCEAPKLKTGVQMIDNGILTFTTMEKEEFLRQEPEAEPLFRKYIGGDEYINGFFRWILYLADAEPSFLRQLPLVQERIRQVRQYRLSSSRPSTVRMADYPTQVGVDERLSGPYLVIPNTSSERRDYVPIGWLTPEVVANQKLRILPDADPWIFGLLTSGMHMAWMRAITGRMKSDYMYSVGVVYNTFPWPDITEAQKQKIRALAQAVLDARALYPGATLADLYDPDLMKRELRQAHRALDAAVDKLYRGQAFANDRERVEHLFGLYEKLSSPLTAAPKPIKRKRKKE

SEQ ID NO: 41 - DNA, 2754 bp, Agmenellum quadruplicatum PR-6
atgcctttaagttggaatgaaatcaaaagtcgggcgatcgccttctcgaaggagtgggaatttgaggagtcagaaaaatcagaagcacaatcgttttggaatgatttttttcaggtatttggcatttctcgtaagcgaatcgcaacatttgagaagtcagttaacaaattagggaataagaaaggttctattgacctgttatggaagggaaatatccttgttgagcataaatcacgaggcaaaagtttagataaggcgtttgaacaggcaaaagattattttccggggttaaaggagcatgagctacctcgatatattttggtgtcggatttcgctcaattccggctttatgacctcgaaacggatcagacccatgaatttctactaaaagatttcgtcaattatgttcatctgtttgattttattgcgggatatgagcagcgaacctataaggatgaagatccggttaatattcacgcggcggagttgatgggtaagctgcatgaccgtctcagggagattggttatacgggtcatgatctagaagtttacttagtgaggttgttattttgcttatttgcagatgacacaggcatttttgaaaagggaatttttgaggaatatctcgatattcataccaaagaagatggtagtgatttggcgatgcacttggggcatattttccatgtgttgaatacgccaccggagaagcggttaaaaaatctggatgagagtttaggacagtttccctatgtgaatggcaagttatttgaagagcagttagcgcctgcggcttttgatcgcaaaatgcgagaaatgttattagaagcttgtggatttaattgggggaaaatttctccggccatttttgggtcaatgttccaagcggcgatggatcaacagactcgacgaaatttgggggcgcattatacgtctgagaaaaatattcagaaggtgattaagcctttgtttttggatgagttgcacgagaaatttaagaaggcaaaaggcagtccaacggcgttaaagcggctccatgatgagcttggggaattacattttcttgatccggcttgtggctgtggaaattttttgattatttcttatcgggaattgcgagatctagagttattgattctcaaagagctttacaagaagaaggaggggtttattgatattcgtttgttcctaaaggtggatgtggatcagtttgggggcattgaatatgatgagtttccggcacgggtggcagaggtggcgatgtggctcatcgatcatcagatgaatatcaaggtgagtaatgagtttgggcagtattttgtccggttgccgctaaagaaggctgccagaattgtgaatgggaatgcgttacggattgattgggaagaagtgattccaaaggaaaagttaaattacattctcggtaatccaccttttgtgggttcaaagatgatgacgaaagatcagcgagcagatcttttatctgtttttgaaagtgccaagggtgcaggggtaatggattatgtttctgcttggtatgttaaagcggcagattttattcaagagaaaaagataaaaacagcttttgtaagtacaaattctatctctcaaggtgagcaagttggaattttatggggactactttttgaaaaatatcaaattaagattcattttgcacaccgtacttttaaatggtcaaatgaggcaaaagggaaagcggctgtttattgtgtgattattggatttgcaacttttaacattaaaggaaagcgtttattcgagtatgaagatatcaagggagaagcgttagaaatcaaagtaagtaacatcaatccatatttggtaaatggtgatgatttaattattctaagacggcggcaacctttatgtaatgtccctaatattggcattggcaataagcccattgatggcggccattacttgttcaccacagaagaaaaggaggattttttaaaactagagccaaaagcagaaaaatggtttaggaaatggttgggttctagggagtttatcaataaagaagaaagatggtgtttgtggttgggagactgtccacctaacgaactcaaaaaaatgccccatgctttagagcgagtcaaggcagttaaagaaactcgattaaatagcaacagtaaaccgacccaaaagctagcgcaaacaccgacaagatttcatgttgaaaatatgccagaatcagaatatttacttattccaaaagtttctagtgaaaggcgcaactatattcctattgggtttttaaatcaaagtacgttatctagtgacttggtgtttattgttggtaatgccaccttgtttcattttggtatctttacttcagtaatgcacatggcatgggttaaatatgtttgtggaagattaaaaagtgattatcgttattcaaaagatattgtctataataattttccttttccgcagaacgtaactgacaaacaaaaacaaacagttgaaaaagcagcgcagttagttttagacactagagacaaatatcccgatagtagccttgccgatctttacgatcccctcaccatgccccccgacttaatgaaagcccaccaaaaactcgataaagcagtggatctctgttaccgtcctcaagcttttaccagcgaactcaaccgcatcgaatttttatttaacgaatatgagaaactgataacaccactcctacaaagtacaaaacagaaaaaagcccgcaaaaacaaaacatcttaa

SEQ ID NO: 42 - protein, 917 aa, Agmenellum quadruplicatum PR-6
MPLSWNEIKSRAIAFSKEWEFEESEKSEAQSFWNDFFQVFGISRKRIATFEKSVNKLGNKKGSIDLLWKGNILVEHKSRGKSLDKAFEQAKDYFPGLKEHELPRYILVSDFAQFRLYDLETDQTHEFLLKDFVNYVHLFDFIAGYEQRTYKDEDPVNIHAAELMGKLHDRLREIGYTGHDLEVYLVRLLFCLFADDTGIFEKGIFEEYLDIHTKEDGSDLAMHLGHIFHVLNTPPEKRLKNLDESLGQFPYVNGKLFEEQLAPAAFDRKMREMLLEACGFNWGKISPAIFGSMFQAAMDQQTRRNLGAHYTSEKNIQKVIKPLFLDELHEKFKKAKGSPTALKRLHDELGELHFLDPACGCGNFLIISYRELRDLELLILKELYKKKEGFIDIRLFLKVDVDQFGGIEYDEFPARVAEVAMWLIDHQMNIKVSNEFGQYFVRLPLKKAARIVNGNALRIDWEEVIPKEKLNYILGNPPFVGSKMMTKDQRADLLSVFESAKGAGVMDYVSAWYVKAADFIQEKKIKTAFVSTNSISQGEQVGILWGLLFEKYQIKIHFAHRTFKWSNEAKGKAAVYCVIIGFATFNIKGKRLFEYEDIKGEALEIKVSNINPYLVNGDDLIILRRRQPLCNVPNIGIGNKPIDGGHYLFTTEEKEDFLKLEPKAEKWFRKWLGSREFINKEERWCLWLGDCPPNELKKMPHALERVKAVKETRLNSNSKPTQKLAQTPTRFHVENMPESEYLLIPKVSSERRNYIPIGFLNQSTLSSDLVFIVGNATLFHFGIFTSVMHMAWVKYVCGRLKSDYRYSKDIVYNNFPFPQNVTDKQKQTVEKAAQLVLDTRDKYPDSSLADLYDPLTMPPDLMKAHQKLDKAVDLCYRPQAFTSELNRIEFLFNEYEKLITPLLQSTKQKKARKNKTS

SEQ ID NO: 43 - DNA, 2745 bp, Agmenellum quadruplicatum PR-6
atggcagtaacccgtgattctctccaggcgtttgtggattactgtaatgcctacatccaaggggatgagaagtcagaggcacagacatttttaacgcgatttttccaagcctttggccatgctgggatcaaggaagttggggccgagtttgaggagcgggtcaaaaaagcgagcaagaaagataaaacaggttttgcggatttggtctggtcgcccgcccctggggtaaagggggtcgtggtggagatgaaaaagcgcgggacagatctggcgctgcattattctcagctcgaaaaatattggctgcggctcaccccgaaaccacgctattcgattctctgtaattttgatgagttttgggtctatgactttaacaaccaggtcgatgagcctgtagaccgggtcaagctagaagatctcccgaaccgggtagggacattttcgtttatggagatcggtggtcgggagccgatctttcggaacaatcaggtcgaggtgacggaacgcacggccaagcgcatgggggaattttatcggctggtgcgatcgcggggcgaaagggaaaagtttgtttatttcacagaagcgcaactgcaacggtttaccctgcaatgtgtgctagcgatgtttgccgaagaccggaatctcctgccacgggatctgtttgtggggttggtgcaggactgtttagcggggcgggataatgcctatgatgcctttagtggtttgtttcgggcgatgaacttgccggggatcgtgccccagggtcgttacaagggggtggattattttaatgggggtttgtttggggaaattcagccgattcccttagaaaagaacgagctagaaattctcgatgtgtgtgcgcgggataattgggcgaatatccgaccgtcgatttttggaaatatttttgagagtgccattgatgcggatgagcgccatgccaggggaattcattacacttctgagaaggatatccggcagattgtgcgcccgacgatcgccgactattgggaagggaaaatcgacgaggcgacgacctacgaagatctcgaaaagctgaagcaggaattacgggaatatcgggtattggatccggcgtgcggttcgggaaatttcctttatgtggcttatcaggagttgaagcggctggaacgggttttgctcaacaaaatctatgagcggcgcaaacggttccagggggaagttttacagcaggaagaaatcgggattgtgacgccgttgcagttttttgggatggatacgaatccgtttgcggtgcagttggcgcgggtgacgatgatgatcgcccggaagattgcgattgataagtttgggttaactgagcctgctttgccgttggattctttggatcaaaatattgtctgccaagatgcgctatttaatgactggccaaaggctgacgcgattatcggcaatccgccttttcttggtggctcaagagtacgtttagagcttggggataaatatgttgaacgaatttttgaaaagttttctgatgttaaggacaaagtagacttttgcgtttattggtttcgtctagcacacgaaaatcttaataaaactggtcgagctggtttagttgggacaaattcaattagtcaaggctttagcagaagggcaagcttagaatatattgtcaataacggcggaattattcacgatgcaatctctacacaggtttggtctggacaagcgaatgtccacgttagcttggttaattggcaatatttaaagcctccagaatatgtcttagatcatgaaattgtcaaaaatataaattcatctttaaagtctgaaacggatgtttccaatgccgttaagctaaaagttaatctgaatcaatctttcaaaggtgtgcaacccacgggaaaagactttctgatttctgagaaaaaagtagaaaattggatccagaaaaatacaaaaaacaatcaagtcttgaaactatttgtatcagcttcagatttagccagcaataaaaatggtgaacccagtcgatggattattgattttaatgatttttctttagaagacgcatctacatacaaagagccttttgatcatgttaatttttttgttaagcctcagcgtgaaaataacagagatcaaaaaactagggaatactggtggttatttccaagagctaggcctgcaatgcgtcaagcaatcgagttactagctctttactttgcagttcctagacattctaaatggtttatttttattccttgtaaattagattggcttcctgctgactcaacaactgttgtggcttcggatgatttttatgtgttgggaattttgacatcagatgttcatcgccaatgggtcaaagcccaaagctcaaccctaaaaggtgatacccgctacacccacaatacctgttttgaaacttttccctttccccagacggcgatcgcaaaactcacccaacagatccgccaagggatgatcgacctccacgaatatcgcaccgcccaaatggaagccaaacaatgggggatcaccaaactttacaacgcctttttcgacgaacccgccagccaactccataaactccacaaaaagctcgatgcccttgtgctcaaagcctacggcttcaaaaaagacgacgacattctcgaaaaacttttagacttgaaccttgccctggccgaaaaagaaaaaaatggcgaaaatatagttggcccctgggcgatcgataacccaccaaaataa

SEQ ID NO: 44 - protein, 914 aa, Agmenellum quadruplicatum PR-6
MAVTRDSLQAFVDYCNAYIQGDEKSEAQTFLTRFFQAFGHAGIKEVGAEFEERVKKASKKDKTGFADLVWSPAPGVKGVVVEMKKRGTDLALHYSQLEKYWLRLTPKPRYSILCNFDEFWVYDFNNQVDEPVDRVKLEDLPNRVGTFSFMEIGGREPIFRNNQVEVTERTAKRMGEFYRLVRSRGEREKFVYFTEAQLQRFTLQCVLAMFAEDRNLLPRDLFVGLVQDCLAGRDNAYDAFSGLFRAMNLPGIVPQGRYKGVDYFNGGLFGEIQPIPLEKNELEILDVCARDNWANIRPSIFGNIFESAIDADERHARGIHYTSEKDIRQIVRPTIADYWEGKIDEATTYEDLEKLKQELREYRVLDPACGSGNFLYVAYQELKRLERVLLNKIYERRKRFQGEVLQQEEIGIVTPLQFFGMDTNPFAVQLARVTMMIARKIAIDKFGLTEPALPLDSLDQNIVCQDALFNDWPKADAIIGNPPFLGGSRVRLELGDKYVERIFEKFSDVKDKVDFCVYWFRLAHENLNKTGRAGLVGTNSISQGFSRRASLEYIVNNGGIIHDAISTQVWSGQANVHVSLVNWQYLKPPEYVLDHEIVKNINSSLKSETDVSNAVKLKVNLNQSFKGVQPTGKDFLISEKKVENWIQKNTKNNQVLKLFVSASDLASNKNGEPSRWIIDFNDFSLEDASTYKEPFDHVNFFVKPQRENNRDQKTREYWWLFPRARPAMRQAIELLALYFAVPRHSKWFIFIPCKLDWLPADSTTVVASDDFYVLGILTSDVHRQWVKAQSSTLKGDTRYTHNTCFETFPFPQTAIAKLTQQIRQGMIDLHEYRTAQMEAKQWGITKLYNAFFDEPASQLHKLHKKLDALVLKAYGFKKDDDILEKLLDLNLALAEKEKNGENIVGPWAIDNPPK

SEQ ID NO: 45 - protein, 36 aa, Methylophilus methylotrophus
  MISC_FEATURE (1)..(36): 1-36 correspond to 788-823 of seq id no. 2
LLSSTMHNCWMRNVGGRLESRYRYSASLVYNTFPWI

SEQ ID NO: 46 - protein, 36 aa, unknown
  Environmental sample Sargasso Sea
VLNSTMHMAWTRAVCGRLESRYQYSVTIVYNNFPWP

SEQ ID NO: 47 - protein, 36 aa, Arcanobacterium pyogenes
  MISC_FEATURE (1)..(36): 1-36 correspond to 830-865 of seq id no. 18
LISSSMFITWQKMIGGRLESRLRFANTLTWNTFPVP

SEQ ID NO: 48 - protein, 36 aa, Neisseria lactamica ST640
  MISC_FEATURE (1)..(36): 1-36 correspond to 824-859 of seq id no. 8
ILSSTMHNAFMRTVAGRLESRYQYSASIVYNNFPFP

SEQ ID NO: 49 - protein, 36 aa, Deinococcus radiodurans
  MISC_FEATURE (1)..(36): 1-36 correspond to 898-933 of seq id no. 36
VLSSRVHVTWALAQGGTLEDRPRYNKTRCFETFPFP

SEQ ID NO: 50 - protein, 36 aa, Rhodopseudomonas palustris BisB5
  MISC_FEATURE (1)..(36): 1-36 correspond to 878-913 of seq id no. 26
VLSSRLHVRWSLSKGGTLEDRPRYNNSMCFDPFPFP

SEQ ID NO: 51 - protein, 36 aa, Deinococcus radiophilus R1
  MISC_FEATURE (1)..(36): 1-36 correspond to 805-840 of seq id no. 22
VIQSSVHWQWLIARGGTLTARLMYTSDTVFDTFPWP

SEQ ID NO: 52 - protein, 36 aa, Marinobacter aquaeolei VT8
  MISC_FEATURE (1)..(36): 1-36 correspond to 882-917 of seq id no. 38
VLSSRVHVLWSLFAGGTLENRPRYNKTLCFETFPFP

SEQ ID NO: 53 - protein, 36 aa, Nitrobacter hamburgensis X14
  MISC_FEATURE (1)..(36): 1-36 correspond to 815-850 of seq id no. 24
ILQSGIHWEWFINRCSTLKADFRYTSDTVFDSFPWP

SEQ ID NO: 54 - protein, 36 aa, Neisseria meningitidis Z2491
  MISC_FEATURE (1)..(36): 1-36 correspond to 798-833 of seq id no. 14
ILSSTMHNAFMRTVAGRLKSDYRYSNTVVYNNFPFP

SEQ ID NO: 55 - protein, 36 aa, Corynebacterium diphtheriae
  MISC_FEATURE (1)..(36): 1-36 correspond to 807-842 of seq id no. 16
VLVSQFQNAWMRVVAGRLKSDYRYGNTTVYNNFVFP

SEQ ID NO: 56 - protein, 36 aa, Agmenellum quadruplicatum PR-6
  MISC_FEATURE (1)..(36): 1-36 correspond to 807-842 of seq id no. 42
IFTSVMHMAWVKYVCGRLKSDYRYSKDIVYNNFPFP

SEQ ID NO: 57 - protein, 36 aa, Corynebacterium striatum M82B
  MISC_FEATURE (1)..(36): 1-36 correspond to 832-867 of seq id no. 12
LASSSMFITWQKSIGGRLKSDLRFANTLTWNTFPVP

SEQ ID NO: 58 - protein, 36 aa, Sulfurimonas denitrificans
  MISC_FEATURE (1)..(36): 1-36 correspond to 773-808 of seq id no. 6
ILTSKMHMDWVRYVAGRLKSDYRYSNEIVYNNFPFP

SEQ ID NO: 59 - protein, 36 aa, Psychrobacter sp. PRwf-1
  MISC_FEATURE (1)..(36): 1-36 correspond to 804-839 of seq id no. 10
TLSSSMHNAFMRLTAGRMKSDYSYSSTIVYNNFPYP

SEQ ID NO: 60 - protein, 36 aa, Parvibaculum lavamentivorans DS-1
  MISC_FEATURE (1)..(36): 1-36 correspond to 786-831 of seq id no. 40
LLTSGMHMAWMRAITGRMKSDYMYSVGVVYNTFPWP

SEQ ID NO: 61 - protein, 38 aa, Silicibacter pomeroyi DSS-3
  MISC_FEATURE (1)..(36): 1-36 correspond to 797-835 of seq id no. 20
ILHSSFHELWSLRMGTFLGVGNDPRYTPSTTFETFPFP

SEQ ID NO: 62 - protein, 36 aa, Agmenellum quadruplicatum PR-6
  MISC_FEATURE (1)..(36): 1-36 correspond to 776-811 of seq id no. 44
ILTSDVHRQWVKAQSSTLKGDTRYTHNTCFETFPFP

SEQ ID NO: 63 - protein, 39 aa, Pseudomonas species OM2164
  MISC_FEATURE (1)..(39): 1-39 correspond to 912-950 of seq id no. 34
IVSSRIHWVWAIANAAKIGMYDGDAVYPKGQCFDPFPFP

SEQ ID NO: 64 - protein, 79 aa, Agmenellum quadruplicatum PR-6
  MISC_FEATURE (1)..(79): 1-79 correspond to 740-818 of seq id no. 42
EYLLIPKVSSERRNYIPIGFLNQSTLSSDLVFIVGNATLFHFGIFTSVMHMAWVKYVCGRLKSDYRYSKDIVYNNFPFP

SEQ ID NO: 65 - protein, 79 aa, Sulfurimonas denitrificans
  MISC_FEATURE (1)..(79): 1-79 correspond to 730-808 of seq id no. 6
DYIFIPRVSSENRDYIPMEFFTKDFICGDTGLAVPNATLFHFGILTSKMHMDWVRYVAGRLKSDYRYSNEIVYNNFPFP

SEQ ID NO: 66 - protein, 79 aa, Psychrobacter sp. PRwf-1
  MISC_FEATURE (1)..(79): 1-79 correspond to 761-839 of seq id no. 10
PYVAIPVVSSENRRFIPIGFIDGNTVAGNKLFVIVDGNTYQFGTLSSSMHNAFMRLTAGRMKSDYSYSSTIVYNNFPYP

SEQ ID NO: 67 - protein, 79 aa, unknown
  Environmental sample Sargasso Sea
PFMVIPEVSSERREFIPLGYLQPPTLASNKLRLMPDATLYHFAVLNSTMHMAWTRAVCGRLESRYQYSVTIVYNNFPWP

SEQ ID NO: 68 - protein, 79 aa, Methylophilus methylotrophus
  MISC_FEATURE (1)..(79): 1-79 correspond to 745-823 of seq idi no. 2
DYLLIPETSSENRQFIPIGFVDRNVISSNATYHIPSAEPLIFGLLSSTMHNCWMRNVGGRLESRYRYSASLVYNTFPWI

SEQ ID NO: 69 - protein, 79 aa, Parvibaculum lavamentivorans DS-1
  MISC_FEATURE (1)..(79): 1-79 correspond to 753-831 of seq id no. 40
PYLVIPNTSSERRDYVPIGWLTPEVVANQKLRILPDADPWIFGLLTSGMHMAWMRAITGRMKSDYMYSVGVVYNTFPWP

SEQ ID NO: 70 - protein, 79 aa, Neisseria lactamica ST640
  MISC_FEATURE (1)..(79): 1-79 correspond to 781-859 of seq id no. 8
RYLLLPKVSSENRRFLPIGYIEPETIANGSALIIPNATLCHFGILSSTMHNAFMRTVAGRLESRYQYSASIVYNNFPFP

SEQ ID NO: 71 - protein, 79 aa, Neisseria meningitidis Z2491
  MISC_FEATURE (1)..(79): 1-79 correspond to 755-833 of seq id no. 14
NYLIIPSVSSESRRFIPIGYLSFETVVSNLAFILPNATLYHFGILSSTMHNAFMRTVAGRLKSDYRYSNTVVYNNFPFP

SEQ ID NO: 72 - protein, 79 aa, Arcanobacterium pyogenes
  MISC_FEATURE: 1-79 correspond to 787-865 of seq id no. 18
DFLCVPSVVSENRPYFTAADIEEGTVVSSLAFAVEDSDRSQFALISSSMFITWQKMIGGRLESRLRFANTLTWNTFPVP

SEQ ID NO: 73 - protein, 79 aa, Corynebacterium striatum M82B
  MISC_FEATURE (1)..(79): 1-79 correspond to 789-867 of seq id no. 12
DYLCLPKVVSERRSYFTVQRYPSNVIASDLVFHAQDPDGLMFALASSSMFITWQKSIGGRLKSDLRFANTLTWNTFPVP

SEQ ID NO: 74 - protein, 79 aa, Corynebacterium diphtheriae
  MISC_FEATURE (1)..(79): 1-79 correspond to 764-842 of seq id no. 16
TYIGIPKVSSERRKYVPFAFVTDGMIPGDMLYFVPTDSLFVFGVLVSQFQNAWMRVVAGRLKSDYRYGNTTVYNNFVFP

SEQ ID NO: 75 - protein, 75 aa, Rhodopseudomonas palustris BisB5
  MISC_FEATURE (1)..(75): 1-75 correspond to 839-913
RYIGTARTAKHRIFSMLAGHSLPESEVIAVGSDDAFILGVLSSRLHVRWSLSKGGTLEDRPRYNNSMCFDPFPFP

SEQ ID NO: 76 - protein, 78 aa, Pseudomonas species OM2164
  MISC_FEATURE (1)..(78): 1-78 correspond to 873-950 of seq id no. 34
RYIATVETAKHRIFSLLDATILPDNKLIIIALADTWHFSIVSSRIHWVWAIANAAKIGMYDGDAVYPKGQCFDPFPFP

SEQ ID NO: 77 - protein, 75 aa, Marinobacter aquaeolei VT8
  MISC_FEATURE (1)..(75): 1-75 correspond to 843-917 of seq id no. 38
TAIATSLTAKHRVFVHLDSNSICDSTTVMFALPGAQYLGVLSSRVHVLWSLFAGGTLENRPRYNKTLCFETFPFP

SEQ ID NO: 78 - protein, 75 aa, Deinococcus radiodurans
  MISC_FEATURE (1)..(75): 1-75 correspond to 859-933 of seq id no. 36
RYVVTLETAKHQVFQFLDSSIVPDSTIVTFGTEDAFHLGVLSSRVHVTWALAQGGTLEDRPRYNKTRCFETFPFP

SEQ ID NO: 79 - protein, 77 aa, Silicibacter pomeroyi DSS-3
  MISC_FEATURE (1)..(77): 1-77 corresponds to 758-834 of seq id no. 20
RFIVTPRVGKHRIFVWLDSNALADSATFIVARDDETTFGILHSSFHELWSLRMGTFLGVGNDPRYTPSTTFETFPFP

SEQ ID NO: 80 - protein, 75 aa, Agmenellum quadruplicatum PR-6
  MISC_FEATURE (1)..(75): 1-75 correspond to 737-811 of seq id no. 44
LYFAVPRHSKWFIFIPCKLDWLPADSTTVVASDDFYVLGILTSDVHRQWVKAQSSTLKGDTRYTHNTCFETFPFP

SEQ ID NO: 81 - protein, 75 aa, Nitrobacter hamburgensis X14
  MISC_FEATURE (1)..(75): 1-75 correspond to 776-850 of seq id no. 24
RYIVCARVTHRPIFEFVSTAIHPNDALSVFALEDDYSFGILQSGIHWEWFINRCSTLKADFRYTSDTVFDSFPWP

SEQ ID NO: 82 - protein, 75 aa, Deinococcus radiophilus R1
  MISC_FEATURE (1)..(75): 1-75 correspond to 766-840 of seq id no. 22
RYIVCSRVTKRQVFEFLDNGIRPSDGLQIFAFEDDYSFGVIQSSVHWQWLIARGGTLTARLMYTSDTVFDTFPWP

SEQ ID NO: 83 - protein, 48 aa, Bacillus stearothermophilus LV
  MISC_FEATURE (1)..(48): 1-48 corresponds to 434-481 of the protein M. BstLVI
YEIWVPHDPSLWDKPKIIFPDISPEPKFFYEDKGSVVDGNCYWIIPKK

SEQ ID NO: 84 - protein, 48 aa, Bacillus aneurinolyticus
  MISC_FEATURE (1)..(48): 1-48 correspond to 437-484 of the protein M.BanIII
YQIWLPQNPDHWALPKILFPDISPEPKFFYEDEGCCIDGNCYWIIPKE

SEQ ID NO: 85 - protein, 48 aa, Bacillus stearothermophilus V
  MISC_FEATURE (1)..(48): 1-48 correspond to 422-469 of the protein M.BstVI
FRTIDRIYPEIVHQPKLLIPDMKNTNHIVKDDGAFYPHHNLYYILPGN

SEQ ID NO: 86 - protein, 48 aa, Xanthomonas holcicola
  MISC_FEATURE (1)..(48): 1-48 correspond to 434-481 of the protein M.XhoI
FRTIDRIYPALAKTPKLLVPDIKGDAHIVYEEGKLYPHHNLYFITANE

SEQ ID NO: 87 - protein, 48 aa, Pseudomonas aeruginosa
  MISC_FEATURE (1)..(48): 1-48 correspond to 392-439 of the protein M.PaeR7I
YRTIDRITPALAARPKLLIPDIKGESHIVFEGGELYPSHNLYYVTSDD

SEQ ID NO: 88 - protein, 43 aa, Xanthomonas amaranthicola
  MISC_FEATURE (1)..(43): 1-43 correspond to 434-476 of the protein M.XamI
WSVGLKAPAPILCTYMARRPPQFTLNACDARHINIAHGLYPRE

SEQ ID NO: 89 - protein, 43 aa, Acinetobacter calcoaceticus SRW4
  MISC_FEATURE (1)..(43): 1-43 correspond to 384-426 of the protein M.AcuI
FVIPSIKLSDALFIRRNNLFPRLILNEAQAYTTDTMHRVFIKQ

SEQ ID NO: 90 - protein, 47 aa, Xanthomonas campestris pv. vesicatoria
  MISC_FEATURE (1)..(47): 1-47 correspond to 476-522 of the protein M.XveI
KPCVLLQRTTAKEQARRLIAAEMPASFIKRHAGVTIENHLNMMIPTV

SEQ ID NO: 91 - protein, 48 aa, Bacillus subtilis
  MISC_FEATURE (1)..(48): 1-48 correspond to 371-418 of the protein M.BsuBI
PNGHYVVVKRFSSKEEKRRIVAGVLTPESVNDPVVGFENGLNVLHYNK

SEQ ID NO: 92 - protein, 48 aa, Providencia stuartii 164
  MISC_FEATURE (1)..(48): 1-48 correspond to 383-430 of the protein M.PstI
PNGIYVLTRRLTAKEEKRRIVASIYYPDIANVDTVGFDNKINYFHANG

SEQ ID NO: 93 - protein, 47 aa, Rhizobium leguminosarum VF39SM
  MISC_FEATURE (1)..(47): 1-47 correspond to 478-524 of the protein M.Rle39B
VPCVLLQRTTSKEQARRLIAAELPEAFIKAHGRVIVENHLNMVKPTA

SEQ ID NO: 94 - protein, 47 aa, Xanthomonas phaseoli; identical to SEQ ID NO: 90
  MISC_FEATURE (1)..(47): 1-47 correspond to 475-521 of the protein M.XphI

SEQ ID NO: 95 - protein, 43 aa, Bacillus pumilus
  MISC_FEATURE (1)..(43): 1-43 correspond to 398-440 of the protein M.BpmI
YITPSRWVPDAFALRQVDGYPKLILNETDASSTDTIHRVRFKE

SEQ ID NO: 96 - protein, 48 aa, Bacillus species R
  MISC_FEATURE (1)..(48): 1-48 correspond to 533-580 of the protein M.BseRI
YMLPRLTGRHKSELFIPRINNLHPKTLLNSNNTVIDANFSTLWVNKET

SEQ ID NO: 97 - protein, 36 aa, Vibrio species 343
  MISC_FEATURE (1)..(36): 1-36 correspond to 434-469 of the protein M.VspI
AEEKLIYKFISSELVFFHDTKKRFILNSANMLVLQD

SEQ ID NO: 98 - protein, 47 aa, Streptococcus faecalis
  MISC_FEATURE (1)..(47): 1-47 correspond to 505-551 of the protein M.SfeI
YEYGRSQALNSHVPKIIFPTNSLNPNFVYFTDYALFNNGYAIYGVNN

SEQ ID NO: 99 - protein, 43 aa, Acinetobacter calcoaceticus
  MISC_FEATURE (1)..(43): 1-43 correspond to 397-439 of the protein M.AccI
YSLENRKPAPIWVSVFNRSGLRFIRNEANISNLTSYHCIIQNK

SEQ ID NO: 100 - protein, 67 aa, Arcanobacterium pyogenes
  MISC_FEATURE (1)..(67): 1-67 are a portion of the amino acid sequence of the protein ApyPI
DIEEGTVVSSLAFAVEDSDRSQFALISSSMFITWQKMIGGRLESRLRFANTLTWNTFPVPELDEKTR

SEQ ID NO: 101 - protein, 66 aa, Neisseria meningitidis Z2491
  MISC_FEATURE (1)..(66): 1-66 are a portion of the amino acid sequence of the protein NmeAIII
YLSFETVVSNLAFILPNATLYHFGILSSTMHNAFMRTVAGRLKSDYRYSNTVVYNNFPFPESCRLP

SEQ ID NO: 102 - protein, 67 aa, Neisseria lactamica ST640
  MISC_FEATURE (1)..(67): 1-67 are a portion of the amino acid sequence of the protein NlaCI
YIEPETIANGSALIIPNATLCHFGILSSTMHNAFMRTVAGRLESRYQYSASIVYNNFPFPENPCRTA

SEQ ID NO: 103 - protein, 67 aa, Sulfurimonas denitrificans
  MISC_FEATURE (1)..(67): 1-67 aer a portion of the amino acid sequence of the protein SdeAI
FFTKDFICGDTGLAVPNATLFHFGILTSKMHMDWVRYVAGRLKSDYRYSNEIVYNNFPFPLEINDKQ

SEQ ID NO: 104 - protein, 67 aa, Chlorobium chlorochromatii CaD3
  MISC_FEATURE (1)..(67): 1-67 are a portion of the amino acid sequence of the protein CchORF1309P
YFSKDNILHNSCSAVPNATLYHFGILTSTMHMVWMRTVCGRIKSDYRYSNNLVYNNFLFPHDISNKQ

SEQ ID NO: 105 - protein, 67 aa, Gramella forsetii KT0803
  MISC_FEATURE (1)..(67): 1-67 are a portion of the amino acid sequence of the protein GfoORF257P
YLPKEVIVSDSAIALPEANLFTFGILNSLMHMMWMNYTCGRLKSDFRYSNTLVYNNFPFPQEVNQNS

SEQ ID NO: 106 - protein, 66 aa, Methylophilus methylotrophus
  MISC_FEATURE (1)..(66): 1-66 are a portion of the amino acid sequence of the protein MmeI
FVDRNVISSNATYHIPSAEPLIFGLLSSTMHNCWMRNVGGRLESRYRYSASLVYNTFPWIQPNEKQ

SEQ ID NO: 107 - protein, 67 aa, Leptospira biflexa phage LE1
  MISC_FEATURE (1)..(67): 1-67 are a portion of the amino acid sequence of the protein LbiLE1ORFAP
FLSSNVIAANDLQIVPNCDLYTFAFLTSRIHNNWTSLTSGRLKSDIRYSVKLSYNNFPWPENPSDKQ

SEQ ID NO: 108 - protein, 67 aa, Psychrobacter sp. PRwf-1
  MISC_FEATURE (1)..(67): 1-67 are a portion of the amino acid sequence of the protein PsPPRI
FIDGNTVAGNKLEVIVDGNTYQFGTLSSSMHNAFMRLTAGRMKSDYSYSSTIVYNNFPYPFMADDHS

SEQ ID NO: 109 - protein, 66 aa, unknown
  Environmental sample Sargasso Sea
YLQPPTLASNKLRLMPDATLYHFAVLNSTMHMAWTRAVCGRLESRYQYSVTIVYNNFPWPSPSDAQ

SEQ ID NO: 110 - protein, 67 aa, Lactobacillus acidophilus NCFM
  MISC_FEATURE (1)..(67): 1-67 are a portion of the amino acid sequence of the protein LacORF332P
YVSKDVIVNNGASFVPDASLYDLGVLTSNMHMAWMRTVCGYFGPSYRYSNRIVYNNFPWPSATDKQK

SEQ ID NO: 111 - protein, 67 aa, unknown
  Environmental sample Sargasso Sea
FLDNNTISTDLNFIIPEATMYHFAILTSNIHMAWMRAVCGRMKSDYRYSANIVYNNFPWPTPTEQQK

SEQ ID NO: 112 - protein, 67 aa, Lactobacillus fermentum
  MISC_FEATURE (1)..(67): 1-67 are a portion of the amino acid sequence of the protein LfeLORF4P
YLGNDIIPTNLATIIPEADHYAFGVLESIVHMAWMRVVAGRKGTSYRYSKNLVYTNFPWPVVDINQK

SEQ ID NO: 113 - protein, 67 aa, Corynebacterium diphtheriae
  MISC_FEATURE (1)..(67): 1-67 are a portion of the amino acid sequence of the protein CdpI
FVTDGMIPGDMLYFVPTDSLFVFGVLVSQFQNAWMRVVAGRLKSDYRYGNTTVYNNFVFPEVDDSVR

SEQ ID NO: 114 - protein, 66 aa, unknown
  Environmental sample Sargasso Sea
FVPEIFCSNKVRLIPNASLYHYGILQSQFHNAWVRIVTGRLKDDYQYSANIDYNNFVWPEPTESQR

SEQ ID NO: 115 - protein, 67 aa, Chlorobium chlorochromatii CaD3
  MISC_FEATURE (1)..(67): 1-67 are a portion of the amino acid sequence of the protein CchORF759P
FLSSNIIISDAAQAIYEAKPWVFGIISSRMHMTWVRAVAGRLKSDYRYSSAICYNTFPFPPITETQK

SEQ ID NO: 116 - protein, 67 aa, Moraxella osloensis
  MISC_FEATURE (1)..(67): 1-67 are a portion of the amino acid sequence of the protein MslORFHP
FYGKDFKASDSNLIVATSEAYLFGILHSKMHMVWVDAVGGKLKTDYRYSAKLCYNTFPFPDITAKQK

SEQ ID NO: 117 - protein, 67 aa, Bacillus subtilis 168
  MISC_FEATURE (1)..(67): 1-67 aa are a portion of the amino acid sequence of the protein BsuMORF677P
LAGADTILSNLIYVIYDAEIYLLGILMSRMHMTWVKAVAGRLKTDYRYSAGLCYNTFPIPELSTRRK

SEQ ID NO: 118 - protein, 67 aa, nitrosococcus oceani
  MISC_FEATURE (1)..(67): 1-67 aa are a portion fo the amino acid sequence of the protein NocAORF28P
IFEEDVIATNLTLIIPDAGLYDFAILSTQMHMDWLRLVGGRLESRYRYSATIVYNTFPWPNATEAQR

SEQ ID NO: 119 - protein, 67 aa, nitrosococcus oceani
  MISC_FEATURE (1)..(67): 1-67 aa are a portion of the amino acid sequence of the protein NocAORF1465P
FYGVDTISSDANQMVPNATPYEFGILTSEMHNDWMRTVAGRLKSDYRYSATLVYNTFPWPEVTDEQR

SEQ ID NO: 120 - protein, 67 aa, Bordetella parapertussis 12822
  MISC_FEATURE (1)..(67): 1-67 aa are a portion of the amino acid sequence of the protein BpaORF1261P
LIPAGDIITDLNFGLFDAELWNASILMSKLHIVWIATVCGKMKSDFRYSNLMGWNTFPVPTLTEKNK

SEQ ID NO: 121 - protein, 67 aa, Xanthomonas campestris pv. vesicatoria str. 85-10
  MISC_FEATURE (1)..(67): 1-67 aa are a portion of the amino acid sequence of the protein XcaVORF2165P
YEPAGTVVSNLAFALYDAPLWNMALIASRLHLVWIASVCGKMKTDFRYSNTLGWNTFPVPTLTEKNK

SEQ ID NO: 122 - protein, 66 aa, Granulibacter bethesdensis CGDNIH1
  MISC_FEATURE (1)..(66): 1-66 aa are a portion of the amino acid sequence of the protein GbeORF1515P
LLPPRSIVTEAFALYDAPLWNMALIASRLHLVWIATVCGKLETRYRYSNTLGWNTFPVPTLTEKNK

SEQ ID NO: 123 - protein, 67 aa, Novosphingobium aromaticivorans DSM 12444
  MISC_FEATURE (1)..(67): 1-67 aa are a portion of the amino acid sequence of the protein NarDORF261P
LKSSGFVSSHTAYMIYGWHPVEFALLNSRLMLVWTETVGGRLGNGMRFSNTIVYNTFPVPSLTDQNK

SEQ ID NO: 124 - protein, 67 aa, Xanthomonas campestris pv. campestris str. 8004
  MISC_FEATURE (1)..(67): 1-67 aa are a portion of the amino acid sequence of the protein Xca8004ORF2076P
LLSKEAIVHNKAFALYDAPLWNFALIVSKMHLVWVAAVCVRLEMRYSYSNTLGWNTFPVPTLTEQNK

SEQ ID NO: 125 - protein, 67 aa, prochlorococcus marinus SS120
  MISC_FEATURE (1)..(67): 1-67 aa are a portion of the amino acid sequence of the protein PmaSSORF630P
IAENGIIIGDRNFAIHDAPLWNIAIISSRLHWLWIATVCVRMRTDFSYSNTLGWNTFYVPKLTEKNM

SEQ ID NO: 126 - protein, 69 aa, Silicibacter pomeroyi DSS-3
  MISC_FEATURE (1)..(69): 1-69 aa are a portion of the amino acid sequence of the protein SpoDI
WLDSNALADSATFIVARDDETTFGILHSSFHELWSLRMGTFLGVGNDPRYTPSTTFETFPFPEGLTPNI

SEQ ID NO: 127 - protein, 67 aa, Azoarus sp. EbN1
  MISC_FEATURE (1)..(67): 1-67 aa are a portion of the amino acid sequence of the protein AspEBORF295P
WMKPPIIPDKNLVVIARADDVTFGVIHSRLHEVWALRMGTSLEDRPRYTSKSTFRTFPFPAGMTPAD

SEQ ID NO: 128 - protein, 69 aa, Caulobacter crescentus
  MISC_FEATURE (1)..(69): 1-69 aa are a portion of the amino acid sequence of the protein CcrMORF826P
WLDARVLPDHKLQVVTLDDDCSFGVLHSRFHEVWALAAGSWHGSGNDPRYTISTTFETFPFPEGLTPNI

SEQ ID NO: 129 - protein, 63 aa, Deinococcus radiophilus R1
  MISC_FEATURE (1)..(63): 1-63 aa are a portion of the amino acid sequence of the protein DraRORF119P
WLPEGTLPDSQVVVIARDDDFIFGVLASTIHRSWARMQGTYMGVGNDLRYTPSTCFETFPVPA

SEQ ID NO: 130 - protein, 62 aa, Deinococcus radiophilus R1
  MISC_FEATURE (1)..(62): 1-62 aa are a portion of the amino acid sequence of the protein DraRI
FLDNGIRPSDGLQIFAFEDDYSFGVIQSSVHWQWLIARGGTLTARLMYTSDTVFDTFPWPED

SEQ ID NO: 131 - protein, 62 aa, Nitrobacter hamburgensis X14
  MISC_FEATURE (1)..(62): 1-62 aa are a portion of the amino acid sequence of the protein NhaXI
FVSTAIHPNDALSVFALEDDYSFGILQSGIHWEWFINRCSTLKADFRYTSDTVFDSFPWPQE

SEQ ID NO: 132 - protein, 38 aa, Methylophilus methylotrophus
  MISC_FEATURE (1)..(38): 1-38 aa are a portion of the amino acid sequence for MmeI
FGLLSSTMHNCWMRNVGGRLESRYRYSASLVYNTFPWI

SEQ ID NO: 133 - protein, 38 aa, unknown
  Environmental sample Sargasso Sea
FAVLNSTMHMAWTRAVCGRLESRYQYSVTIVYNNFPWP

SEQ ID NO: 134 - protein, 38 aa, Arcanobacterium pyogenes
  MISC_FEATURE (1)..(38): 2-38 aa are a portion of the amino acid sequence of ApyPI
FALISSSMFITWQKMIGGRLESRLRFANTLTWNTFPVP

SEQ ID NO: 135 - protein, 38 aa, Neisseria lactamica ST640
  MISC_FEATURE (1)..(38): 1-38 aa are a portion of the amino acid sequence of NlaCI
FGILSSTMHNAFMRTVAGRLESRYQYSASIVYNNFPFP

SEQ ID NO: 136 - protein, 38 aa, Deinococcus radiodurans
  MISC_FEATURE (1)..(38): 1-38 aa are a portion of the amino acid sequence of DrdIV
LGVLSSRVHVTWALAQGGTLEDRPRYNKTRCFETFPFP

SEQ ID NO: 137 - protein, 38 aa, Rhodopseudomonas palustris BisB5
  MISC_FEATURE (1)..(38): 1-38 aa are a portion of the amino acid sequence of RpaB5I
LGVLSSRLHVRWSLSKGGTLEDRPRYNNSMCFDPFPFP

SEQ ID NO: 138 - protein, 38 aa, Deinococcus radiophilus R1
  MISC_FEATURE (1)..(38): 1-38 aa are a portion of the amino acid sequence for DraRI
FGVIQSSVHWQWLIARGGTLTARLMYTSDTVFDTFPWP

SEQ ID NO: 139 - protein, 38 aa, Marinobacter aquaeolei VT8
  MISC_FEATURE (1)..(38): 1-38 aa are a portion of the amino acid sequence of MaqI
LGVLSSRVHVLWSLFAGGTLENRPRYNKTLCFETFPFP

SEQ ID NO: 140 - protein, 38 aa, Nitrobacter hamburgensis X14
  MISC_FEATURE (1)..(38): 1-38 aa are a portion of the amino acid sequence of NhaXI
FGILQSGIHWEWFINRCSTLKADFRYTSDTVFDSFPWP

SEQ ID NO: 141 - protein, 38 aa, Neisseria meningitidis Z2491
  MISC_FEATURE (1)..(38): 1-38 aa are a portion of the amino acid sequence of NmeAIII
FGILSSTMHNAFMRTVAGRLKSDYRYSNTVVYNNFPFP

SEQ ID NO: 142 - protein, 38 aa, Corynebacterium diphtheriae
  MISC_FEATURE (1)..(38): 1-38 aa are a portion of the amino acid sequence of CdpI
FGVLVSQFQNAWMRVVAGRLKSDYRYGNTTVYNNFVFP

SEQ ID NO: 143 - protein, 38 aa, Agmenellum quadruplicatum PR-6
  MISC_FEATURE (1)..(38): 1-38 aa are a portion of the amino acid sequence of AquIII
FGIFTSVMHMAWVKYVCGRLKSDYRYSKDIVYNNFPFP

SEQ ID NO: 144 - protein, 38 aa, Corynebacterium striatum M82B
  MISC_FEATURE (1)..(38): 1-38 aa are a portion of the amino acid sequence of CstMI
FALASSSMFITWQKSIGGRLKSDLRFANTLTWNTFPVP

SEQ ID NO: 145 - protein, 38 aa, Sulfurimonas denitrificans
  MISC_FEATURE (1)..(38): 1-38 aa are a portion of the amino acid sequence of SdeAI
FGILTSKMHMDWVRYVAGRLKSDYRYSNEIVYNNFPFP

SEQ ID NO: 146 - protein, 38 aa, Psychrobacter sp. PRwf-1
  MISC_FEATURE (1)..(38): 1-38 aa are a portion of the amino acid sequence for PspPRI
FGTLSSSMHNAFMRLTAGRMKSDYSYSSTIVYNNFPYP

SEQ ID NO: 147 - protein, 38 aa, Parvibaculum lavamentivorans DS-1
  MISC_FEATURE (1)..(38): 1-38 aa are a portion of the amino acid sequence for PlaDI
FGLLTSGMHMAWMRAITGRMKSDYMYSVGVVYNTFPWP

SEQ ID NO: 148 - protein, 40 aa, Silicibacter pomeroyi DSS-3
  MISC_FEATURE (1)..(40): 1-40 aa are a portion of the amino acid sequence of SpoDI
FGILHSSFHELWSLRMGTFLGVGNDPRYTPSTTFETFPFP

SEQ ID NO: 149 - protein, 38 aa, Agmenellum quadruplicatum PR-6
  MISC_FEATURE (1)..(38): 1-38 aa are a portion of the amino acid sequence of AquIV
LGILTSDVHRQWVKAQSSTLKGDTRYTHNTCFETFPFP

SEQ ID NO: 150 - protein, 41 aa, Pseudomonas species OM2164
  MISC_FEATURE (1)..(41): 1-41 aa are a portion of the amino acid sequence of PspOMII
FSIVSSRIHWVWAIANAAKIGMYDGDAVYPKGQCFDPFPFP

SEQ ID NO: 151 - DNA, 30 bp, artificial
  primer
ctgacgtatcatattcctagtgctgaacct

SEQ ID NO: 152 - DNA, 32 bp, artificial
  primer
gttacttgaaatgacatttctatcaacaaaac

SEQ ID NO: 153 - DNA, 30 bp, artificial
  primer
aagacgtatcatattcctagtgctgaacct

SEQ ID NO: 154 - DNA, 32 bp, artificial; identical to SEQ ID NO: 152
  primer

SEQ ID NO: 155 - DNA, 25 bp, artificial
  primer
agctattctgccagcctggtttaca

SEQ ID NO: 156 - DNA, 28 bp, artificial
  primer
gtaacgactttctaaccttcctcctaca

SEQ ID NO: 157 - DNA, 63 bp, artificial
  primer
caattggaataaattgtctgttttcagatgatgtgcgaggtatcaacagatagtccgtatccg

SEQ ID NO: 158 - DNA, 60 bp, artificial
  primer
gttttgttgatagaaatgtcatttcaagtgacgcaacgtatcatattcctagtgctgaac

SEQ ID NO: 159 - DNA, 33 bp, artificial
  primer
gctgcctaaccttcctcctacatttctcatcca

SEQ ID NO: 160 - DNA, 31 bp, artificial
  primer
acctatagatattctgccagcctggtttaca

SEQ ID NO: 161 - DNA, 33 bp, artificial
  primer
gtgcctatagatattctgccagcctggtttaca

SEQ ID NO: 162 - DNA, 31 bp, artificial
  primer
tccataaccttcctcctacatttctcatcca

SEQ ID NO: 163 - DNA, 36 bp, artificial
  primer
cgttattcaaatgaaattgtttataacaacttccct

SEQ ID NO: 164 - DNA, 35 bp, artificial
  primer
gtaacgactttctaatcttccagcaacataccgca

SEQ ID NO: 165 - DNA, 29 bp, artificial
  primer
cgatattctgccagcctggtttacaacac

SEQ ID NO: 166 - DNA, 42 bp, artificial
  primer
gtaactagtacctaaccttcctcctacatttctcatccagca

SEQ ID NO: 167 - DNA, 29 bp, artificial
  primer
cgatattctgccagcctggtttacaacac

SEQ ID NO: 168 - DNA, 42 bp, artificial
  primer
gtaaccgttacctaaccttcctcctacatttctcatccagca

(19) United States
(12) Patent Application Publication
(10) Pub. No.: US 2009/0036320 A1
(43) Pub. Date: Feb. 5, 2009

(54) Rational Design of Binding Proteins That Recognize Desired Specific Sequences

(75) Inventor: Richard D. Morgan, Middleton, MA (US)

Correspondence Address: HARRIET M. STRIMPEL, D. Phil., New England Biolabs, Inc., 240 COUNTY ROAD, IPSWICH, MA, 01938-2723, US

(73) Assignee: New England Biolabs, Inc., Ipswich, MA (US)

(21) Appl. No.: 12/143,498

(22) Filed: Jun. 20, 2008

Related U.S. Application Data
(60) Provisional application No. 60/936,504, filed on Jun. 20, 2007.

(51) Int. Cl. C40B 30/02; C12N 9/00; C40B 60/08

(52) U.S. Cl. 506/8; 435/183; 506/37

(57) ABSTRACT

Methods and compositions are provided for creating a binding protein that recognizes a rationally chosen recognition sequence in which a first amino acid has been substituted for a second amino acid using site-directed mutagenesis of a member protein of a set of proteins at an identified position or positions correlated with recognition of a chosen specified target module in the recognition sequence. A system is provided for automating the storage and manipulation of the correlations between positions and types of amino acid residues in the binding protein with specific modules at specified positions in the target recognition sequence and for designing and creating proteins with novel specificities.

### BACKGROUND

A long standing goal of molecular biotechnology has been the ability to design and generate DNA binding proteins that specifically bind at a DNA sequence of choice, rather than rely on the limited set of DNA sequences bound by those proteins identified from nature. To this end, the structures of a number of DNA binding proteins complexed with their DNA target sequence have been determined by crystallography (Lukacs, et al. Nat. Struct. Biol. 7: 134-140 (2000) and the amino acid residues conferring specific DNA base recognition have been determined (Pingoud, et al. Nucleic Acids Res. 29:3705-3727 (2001)). However, to date, rational design experiments in which specific amino acid residues are altered to form DNA binding proteins having new, predetermined specificities have been unsuccessful. For example, attempts to generate restriction endonucleases with new DNA recognition specificities have not achieved their desired goals. As a result, methods have been designed that depend on random alteration of a DNA binding protein, followed by a selection from the pool of randomly altered proteins for those proteins that may bind a differing DNA sequence. Often such attempts result in proteins that bind a relaxed specificity relative to the starting protein or have lowered specificity toward their target DNA binding sequence as compared with similar, non-target DNA sequences.

Nonetheless, an effective method of rational design of binding proteins would permit the expansion of the number of unique recognition sequences that could be bound and acted upon to generate a biological event.

### SUMMARY

Embodiments of the invention provide a method for identifying relationships between selected amino acid residues at specific positions in a binding protein and a module in a recognition sequence to which the binding protein binds. The method involves creating a set of binding proteins using an initial binding protein to query a database in a BLAST search. The properties of each binding protein includes a defined amino acid sequence, the amino acid sequences in the set sharing an expectation value (E) of less than e-20 for sequences of more than 200 amino acids or less than e-10 for sequences of less than 200 amino acids in the BLAST search results. The binding proteins additionally bind to specific target recognition sequences in a substrate that contain position-specific modules. The method further includes aligning the amino acid sequences in the set of proteins. The target recognition sequences recognized by the binding proteins in the set are also aligned where this may occur by means of a position dependent feature in the specific target recognition sequence. Correlations between the aligned position-specific modules in the recognition sequences and one or more position-specific amino acids in the aligned amino acid sequences of the binding proteins are identified.

In an additional embodiment of the invention, a method is provided for expanding the set of binding proteins by using a member of the set of binding proteins to query a database in an additional BLAST search.

In an additional embodiment of the invention, a method is provided for identifying the type and location of an amino acid residue or amino acid residues in a plurality of the binding proteins in the set that determines recognition of one or more position-specific modules in the recognition sequence. The type and location of amino acid residue may be recorded in a catalog along with the association with one or more position-specific modules in one or more aligned recognition sequences of the set of binding proteins. This catalog may be used to rationally modify the amino acid sequence of the aligned binding proteins to recognize an altered specific target recognition sequence. Rational modification of the amino acid sequences may be achieved by mutating non-randomly one or more amino acids at correlated positions in a single binding protein to cause a predictable change in the specific target recognition sequence of the binding protein.

In an additional embodiment of the invention, a method is provided wherein a binding protein member of the set has a known amino acid sequence but an uncharacterized specific target recognition sequence. The method involves the steps of identifying position-specific modules in the recognition sequence by (i) reviewing the alignment of the amino acid sequence of the binding protein member in the aligned set of binding proteins; (ii) reading out amino acid residues at the positions recorded in the catalog; and (iii) comparing the amino acid residues in the binding protein member to the amino acid residues recorded in the catalog so as to determine the specific target recognition sequence of the binding protein member.

In an additional embodiment, each position-specific module is one or more nucleotides in a DNA substrate. Additionally, the set of binding proteins may be a set of DNA binding proteins such as MmeI-like proteins.

In an additional embodiment of the invention, a method is provided for altering the DNA recognition sequence of an MmeI-like DNA binding protein by changing the amino acid residues at a predetermined position or positions in the amino acid sequence of MmeI or an equivalent aligned position or positions in an MmeI-like DNA binding protein. An example of predetermined positions as targets of amino acid modification in Mme I binding protein are any of positions 751+773, 806+808, 774+810, 774, 774+810+809 and 809. Changes in these predetermined positions may further comprise a change in one or more of the nucleotides recognized at one or more of positions at 3, 4 and 6 of the DNA recognition sequence.

An embodiment of the invention provides a method for generating a binding protein, which recognizes a rationally chosen recognition sequence that includes substituting a first amino acid with a second amino acid using site-directed mutagenesis of a member protein of a set of proteins at an identified position or positions correlated with recognition of a chosen specified target module.

An embodiment of the invention provides a method of automating the above that includes: storing amino acid sequences for the binding proteins in a database in a computer-readable memory and performing one or more of the above steps by executing instructions stored in a computer. More particularly, a method is provided for automating one or more functions described in FIG. 25A in boxes 1, 2, 3, 4, 6, and 7B. An additional method is provided for automating one or more steps in FIG. 25B such that steps requiring wet chemistry are performed by a device capable of performing wet chemistry that is linked to a computer.

An embodiment of the invention provides a composition of an MmeI-like enzyme having a mutation resulting in at least one altered amino acid residue at a predetermined position that has a specificity for a DNA recognition sequence that is different by at least one base compared with the DNA recognition sequence of the unaltered enzyme. The difference in at least one base may be a difference in length of the recognition sequence that corresponds to an addition or deletion of a nucleotide from the recognition sequence or corresponds to an alternative recognized nucleotide at a specific position.

An embodiment of the invention provides a system that includes a memory for storing instructions and a computer for executing the instructions, which when executed create a set of binding proteins using an initial binding protein to query a database in a BLAST search, wherein each binding protein has a defined amino acid sequence, the amino acid sequences sharing an expectation value (E) of less than e-20 for sequences of more than 200 amino acids or less than e-10 for sequences of less than 200 amino acids; the binding proteins binding to specific target recognition sequences in a substrate, the target recognition sequences containing position-specific modules. The system may additionally include instructions, which when executed align the specific target recognition sequences recognized by the binding proteins; and align the amino acid sequences of the binding proteins of the set. The system may additionally include instructions which when executed identify correlations between the aligned position-specific modules in the recognition sequences and one or more position-specific amino acids in the aligned amino acid sequences of the binding proteins. The system may further include a means for receiving data from a device for protein synthesis and protein binding analysis and containing instructions, which when executed use the data to validate the correlations by confirming a prediction of binding to a predetermined recognition sequence by a mutated protein; and organize the data into a catalog of validated amino acid or amino acids at identified positions that determine recognition for a position and type of module in the recognition sequence.

In another embodiment of the invention, a system is provided which has a memory for storing instructions and a computer for executing the instructions, which when executed, (a) collect and align a sorted set of amino acid sequences of binding proteins in a first database, and collect and align a sorted set of recognition sequences for at least a subset of the binding proteins in a second database, wherein the first database is obtained from an automated search of a third database of amino acid or nucleotide sequences; (b) identify correlations between amino acids at selected aligned positions in the set of amino acid sequences and modules at selected aligned positions of modules in the recognition sequences; (c) from an instrument for protein synthesis and protein binding analysis receive data on the correlations for using the data to validate the correlations by confirming a prediction of binding to a predetermined recognition sequence by a mutated protein; and (d) organize the data into a catalog of validated amino acid or amino acids at identified positions that determine recognition for a position and type of module in the recognition sequence.

In an additional embodiment of the invention, a system is provided having a memory for storing instructions and a computer for executing the instructions that stores positional information on one or more amino acid residues in a first binding protein for targeted mutation to create a second binding protein having a predicted alteration of a module in a sequence position within a sequence of modules recognized by the protein. An example of such stored instructions is provided in FIG. 7A.

### BRIEF DESCRIPTION OF THE DRAWINGS

FIG. 1 shows the cleavage activity of rationally altered MmeI E806K+R808D.

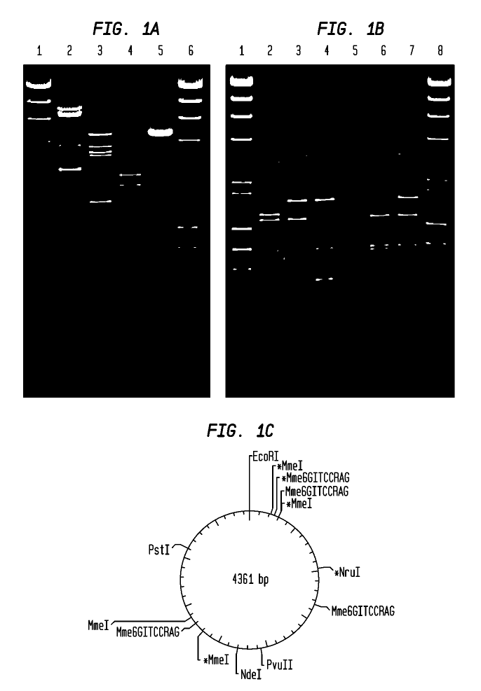

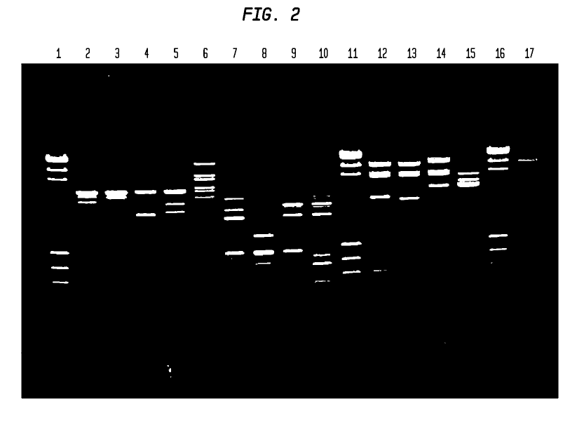

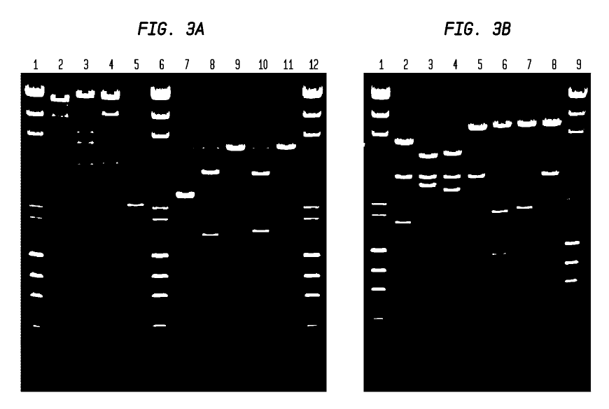

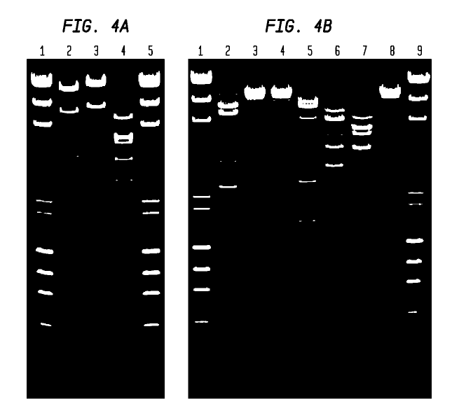

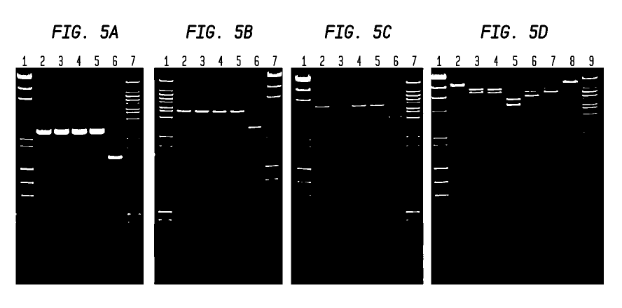

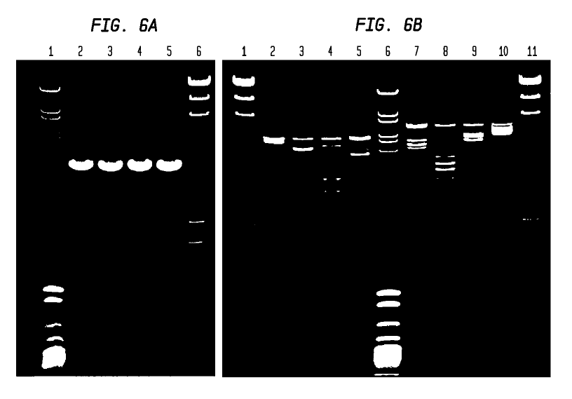

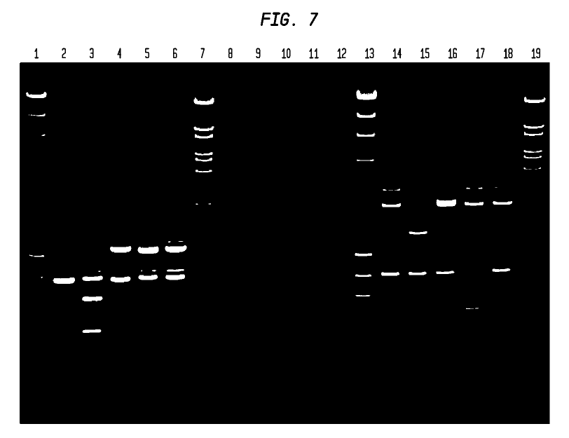

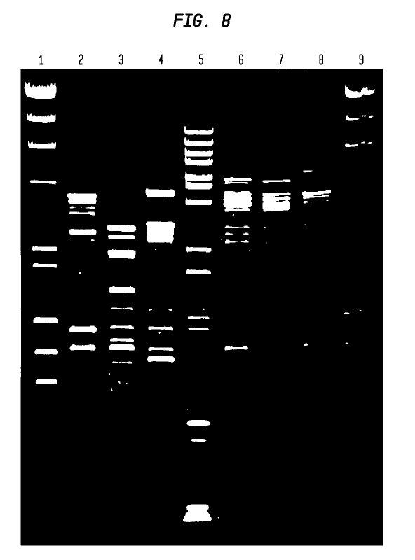

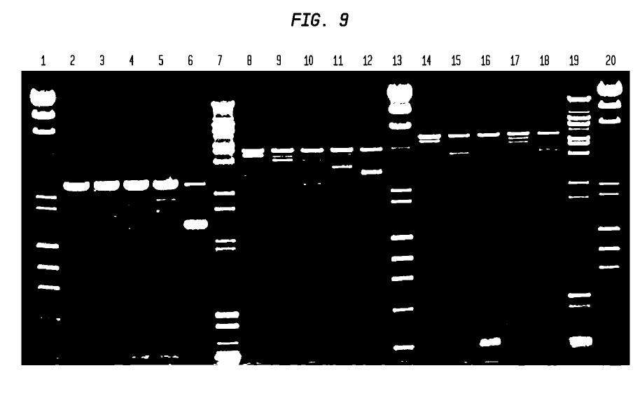

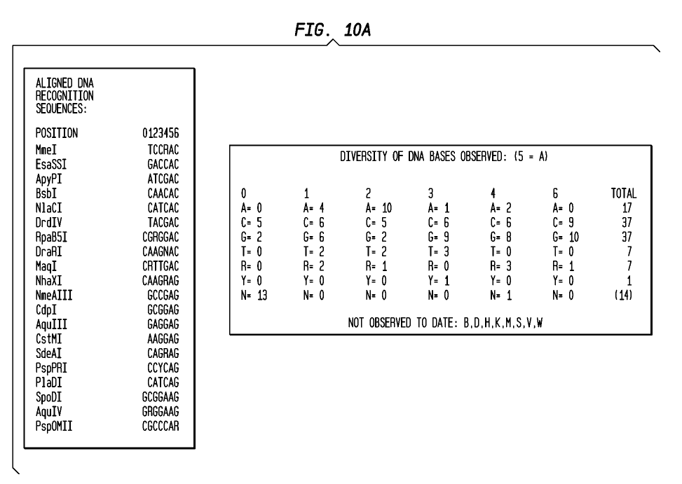

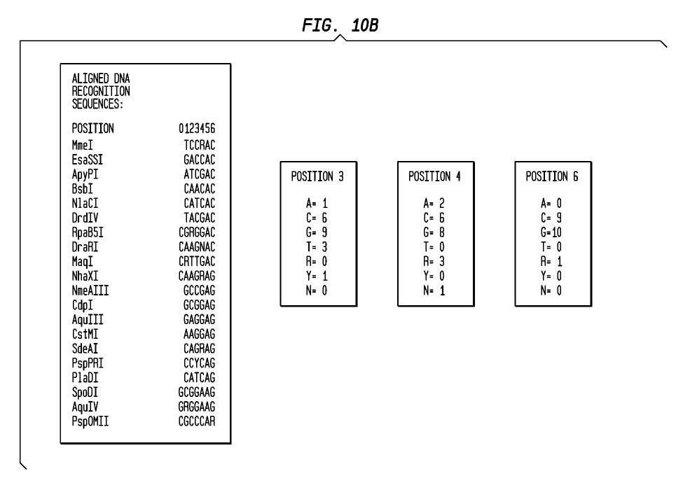

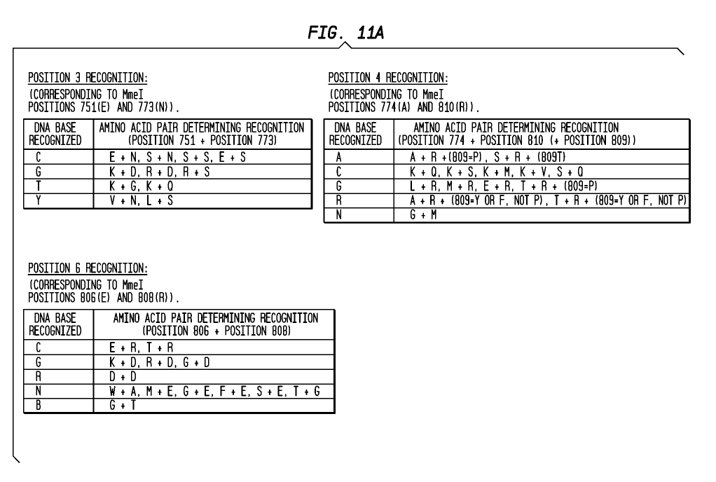

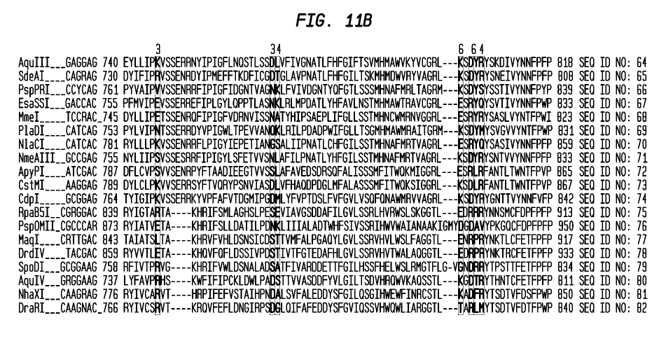

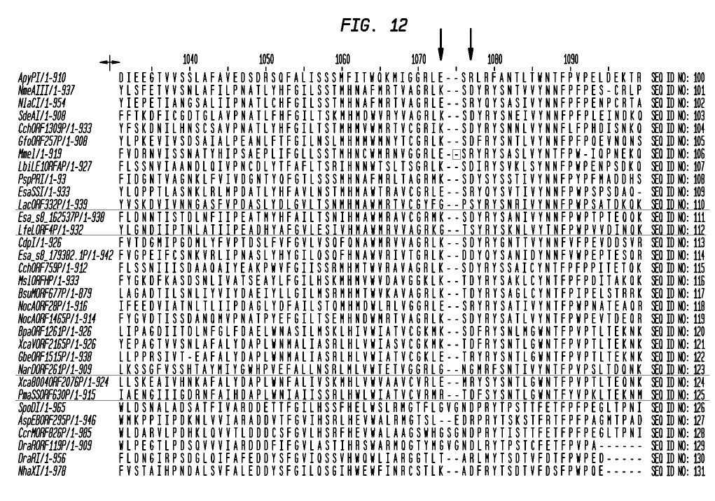

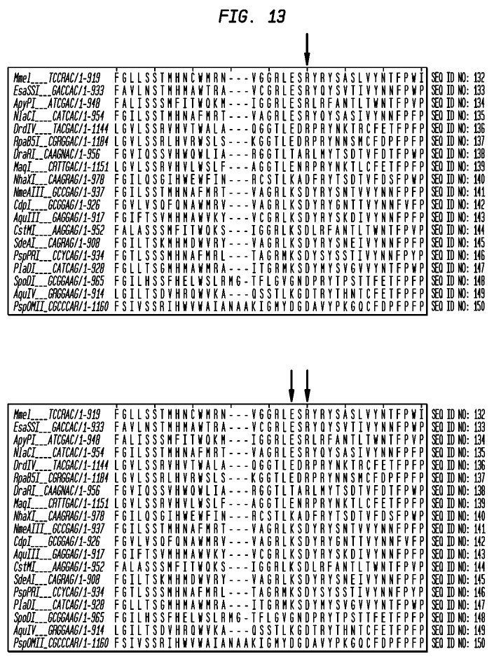

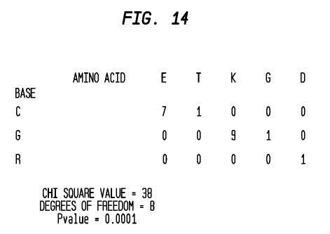

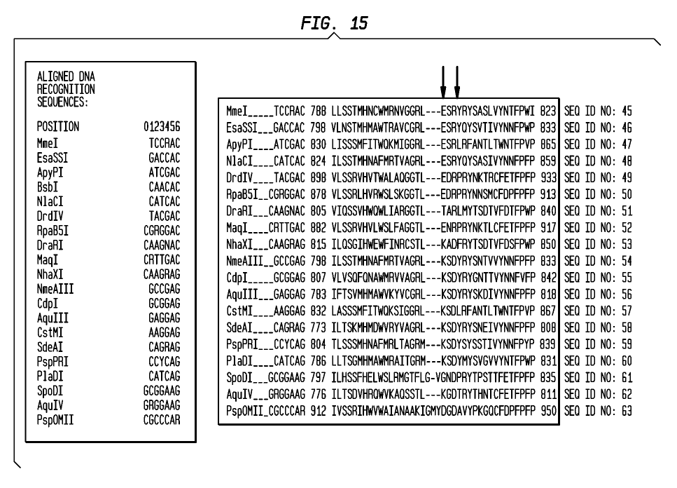

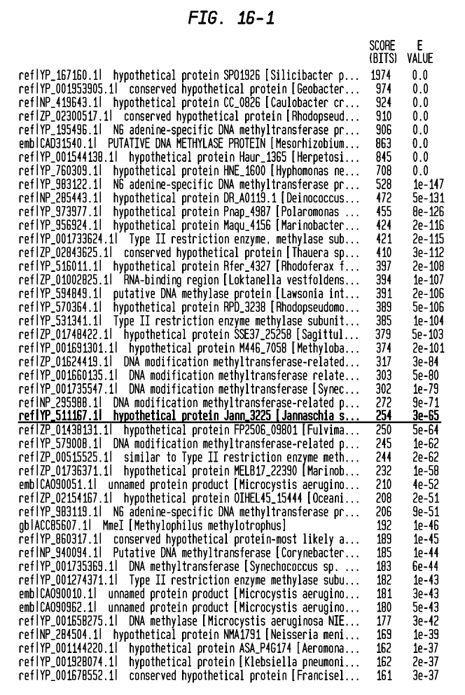

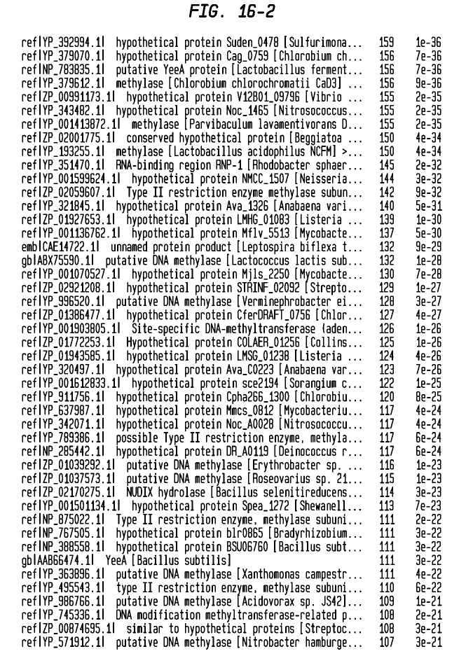

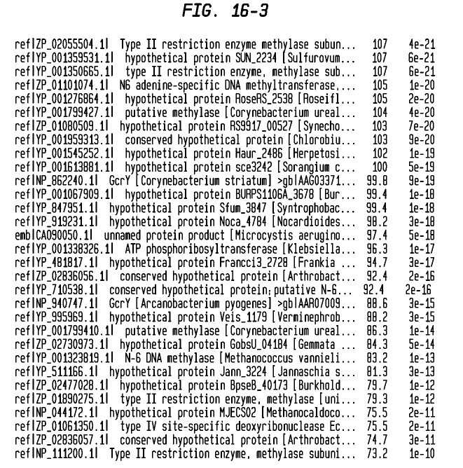

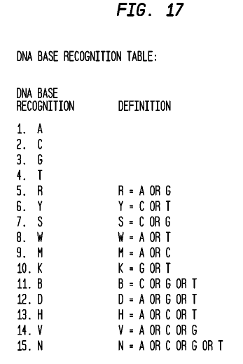

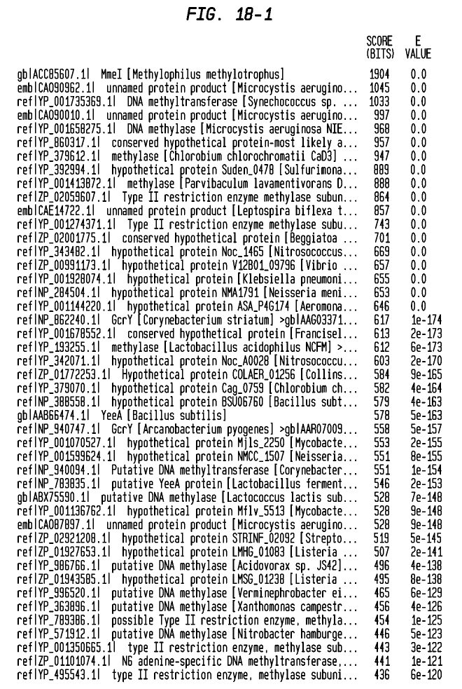

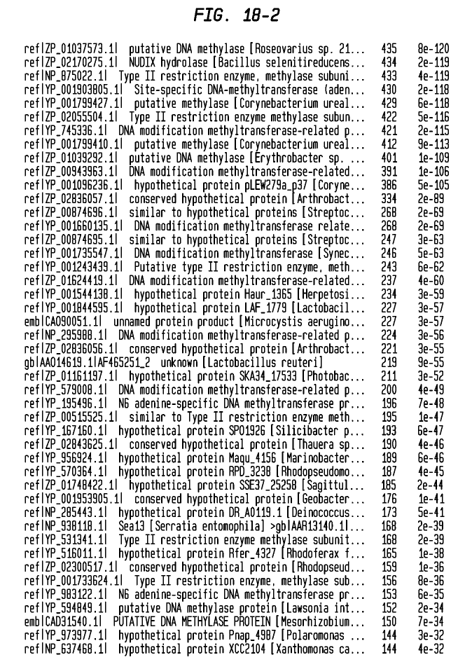

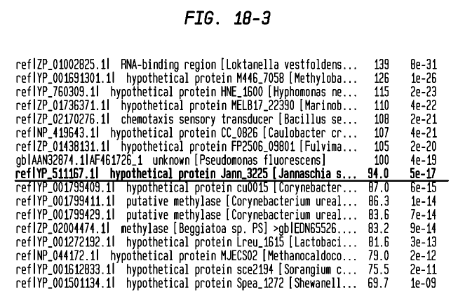

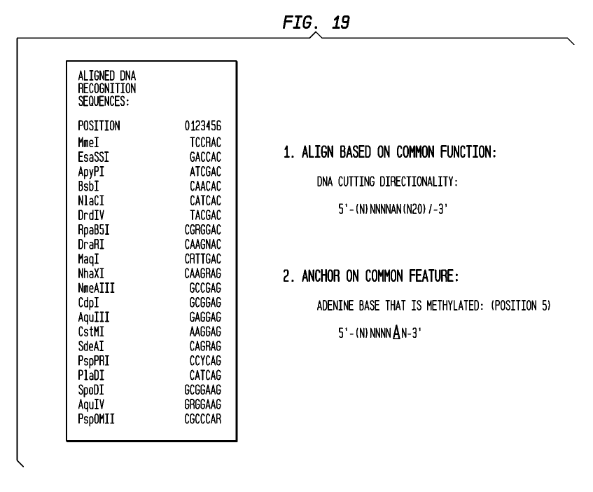

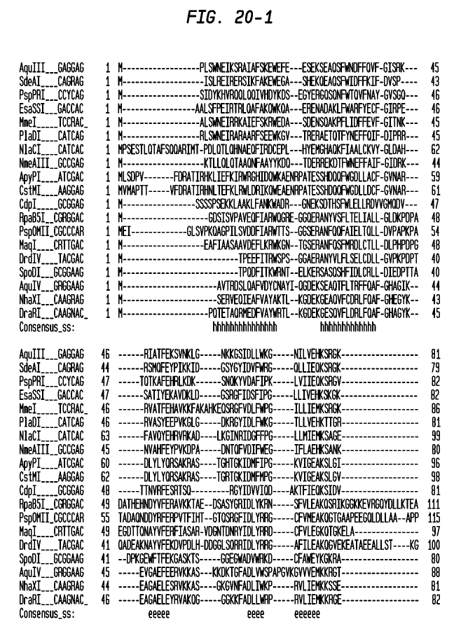

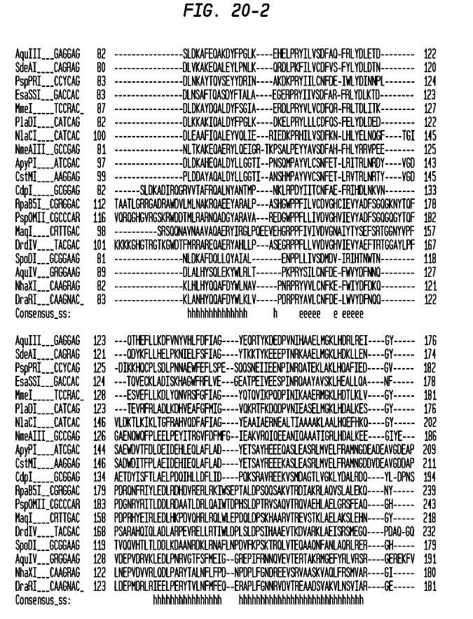

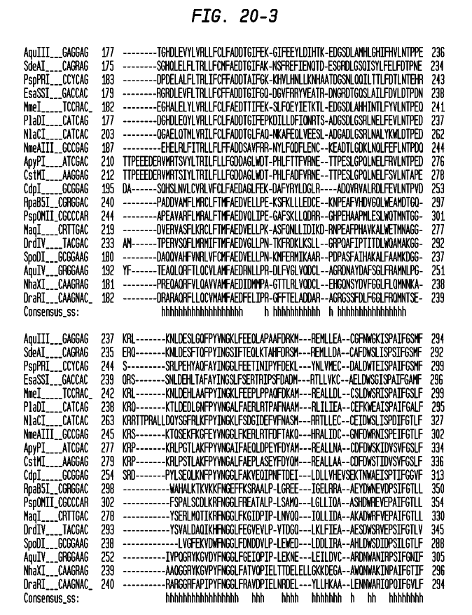

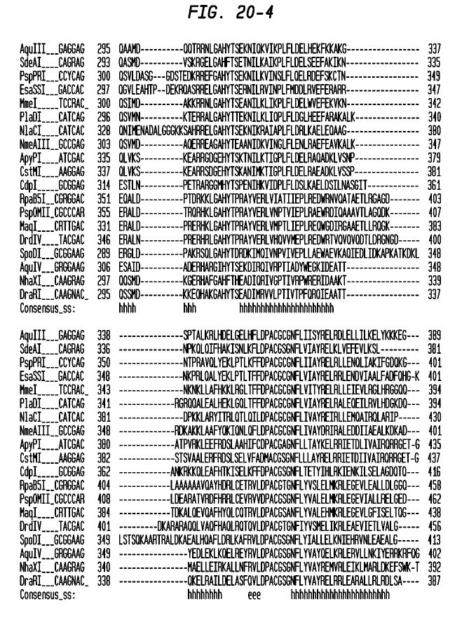

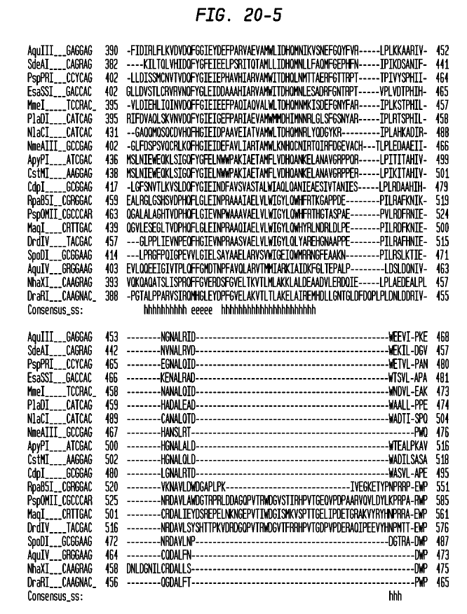

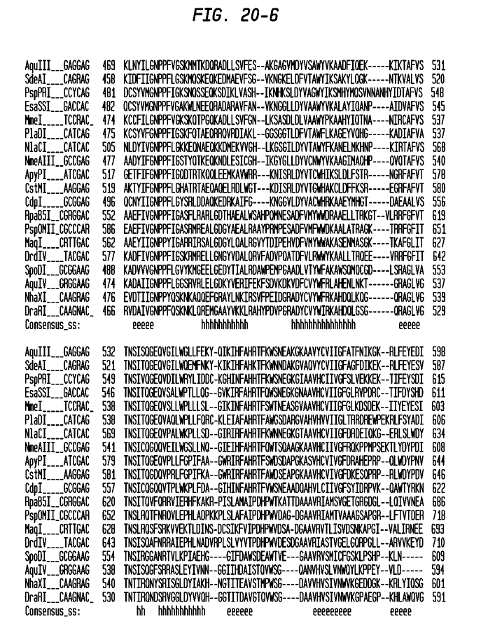

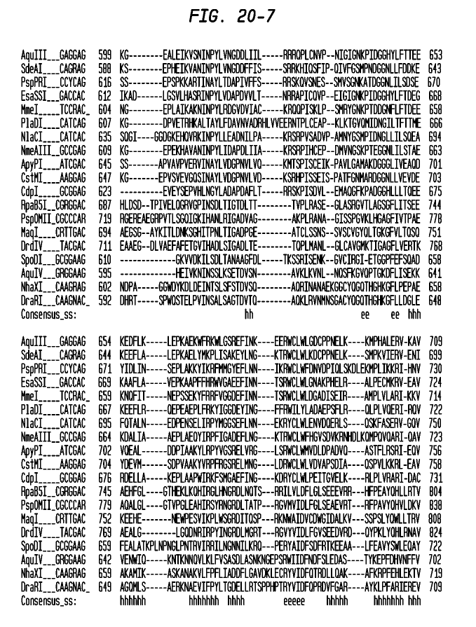

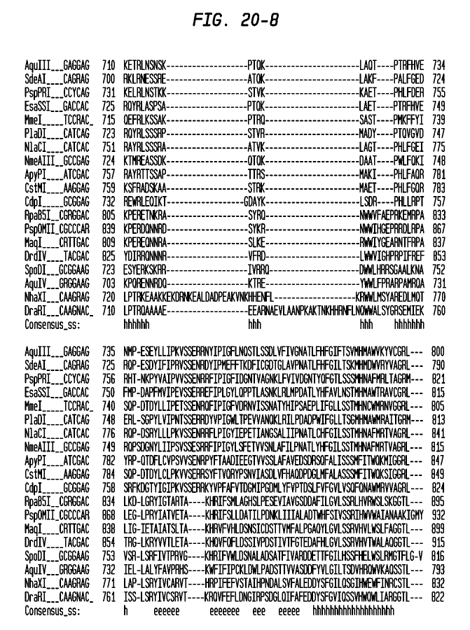

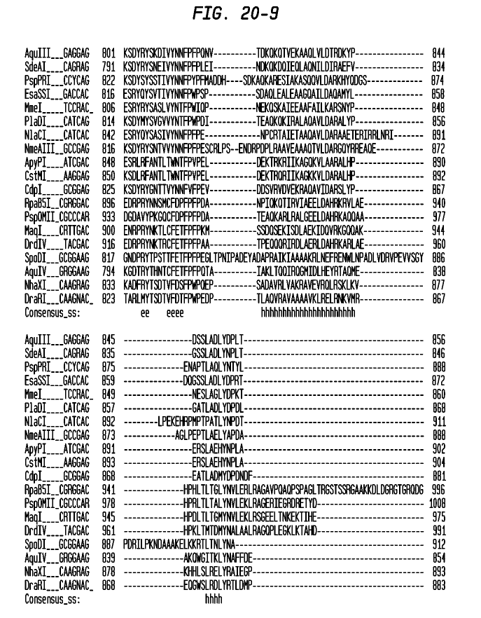

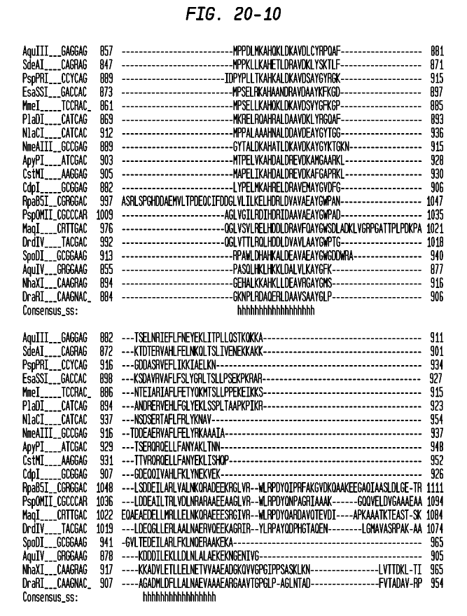

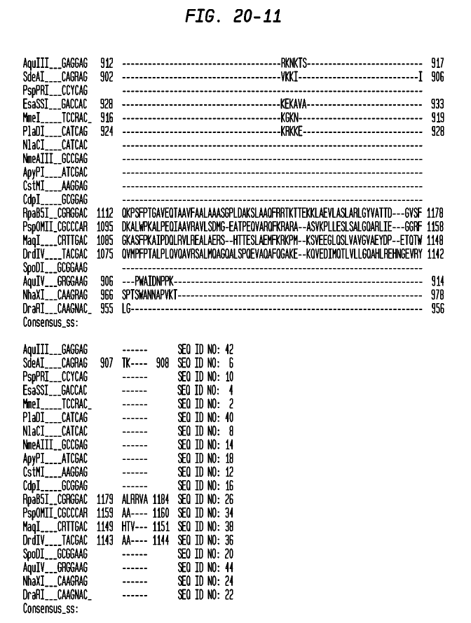

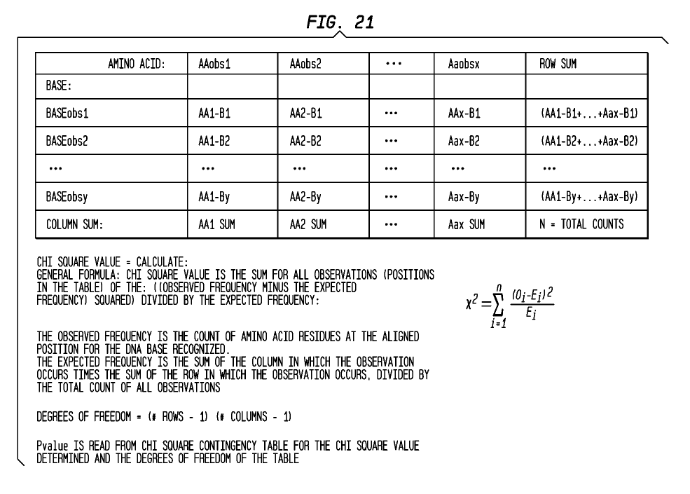

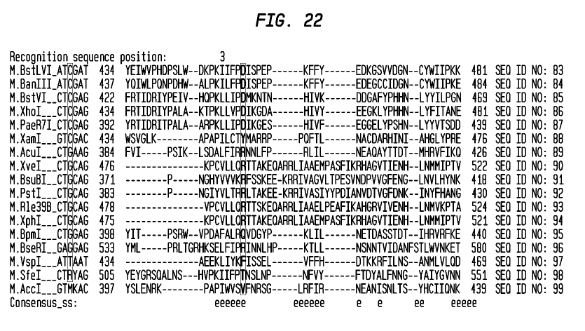

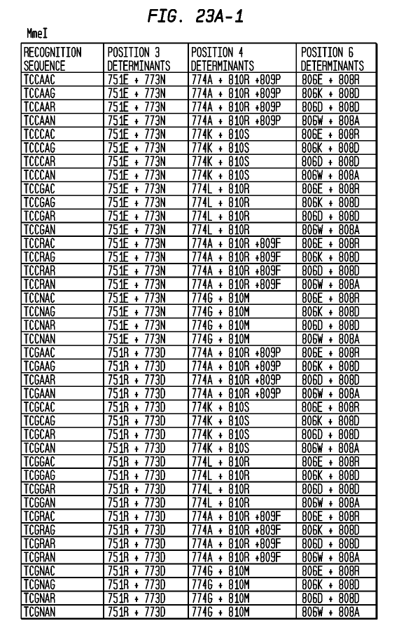

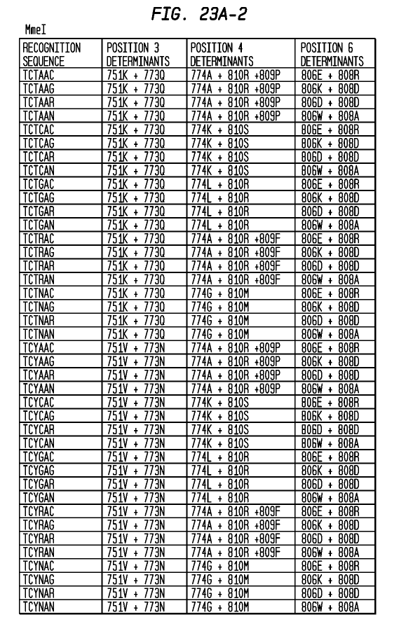

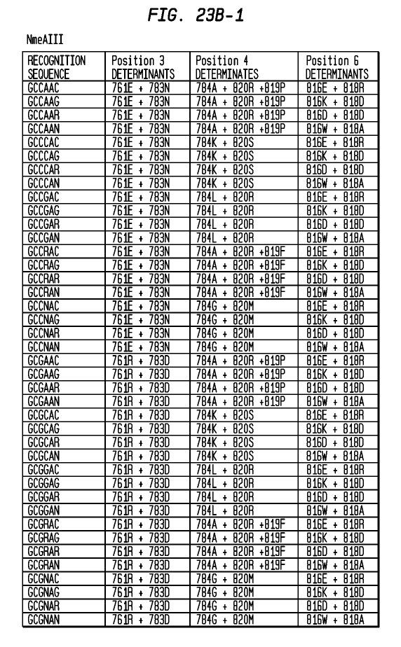

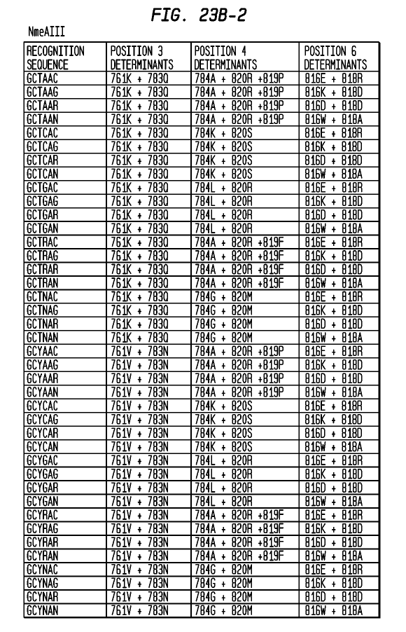

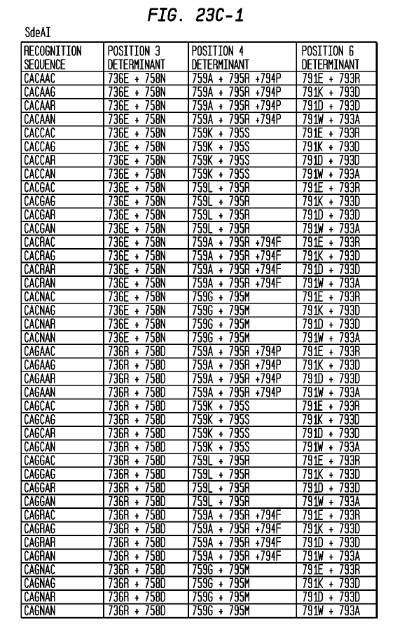

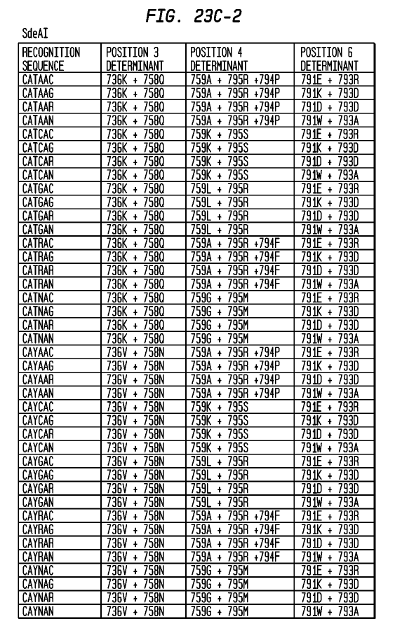

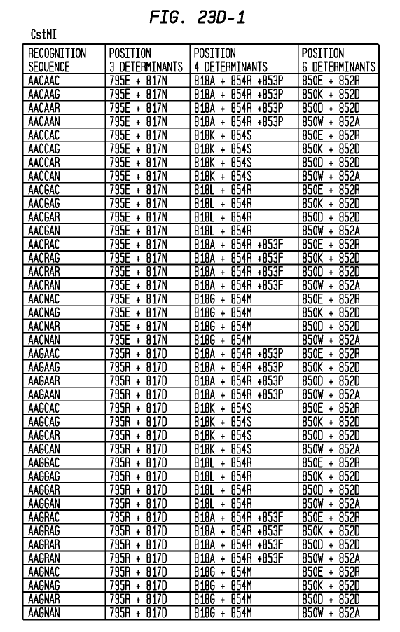

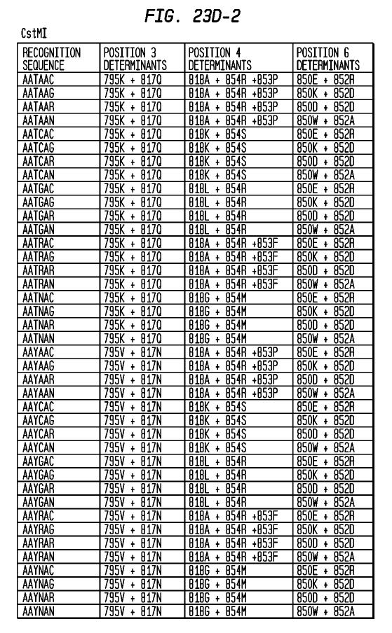

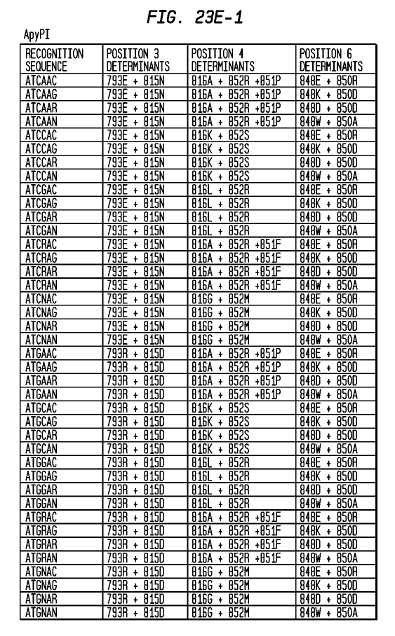

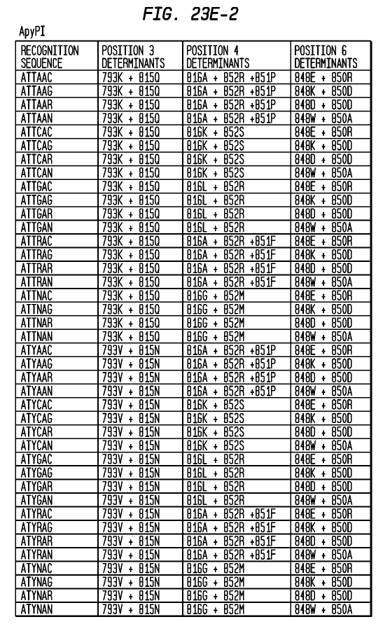

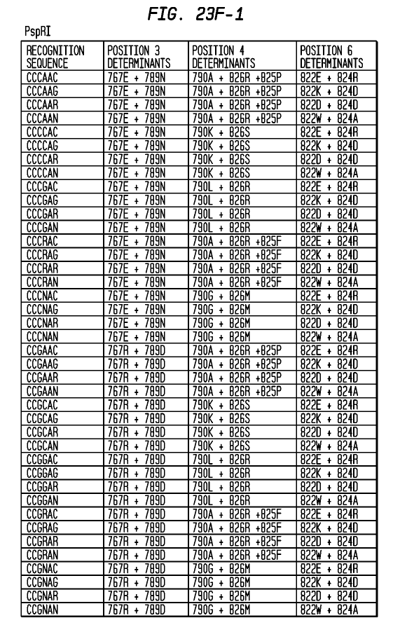

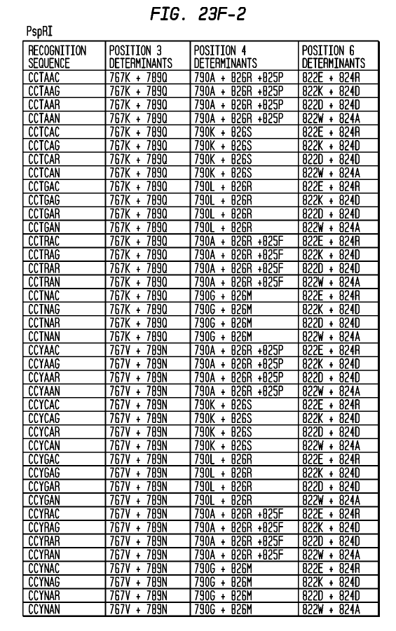

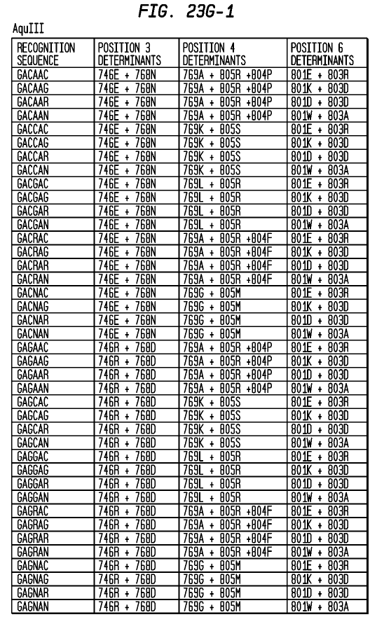

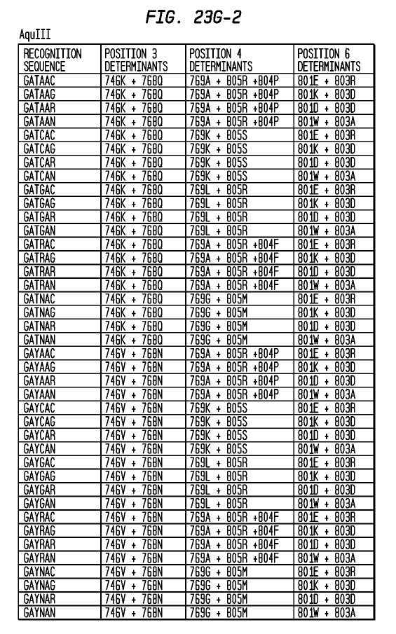

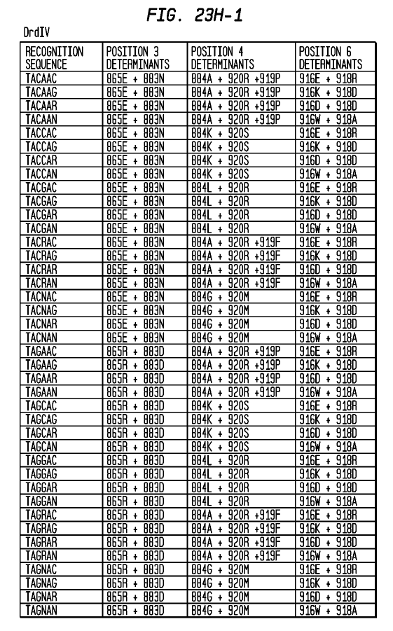

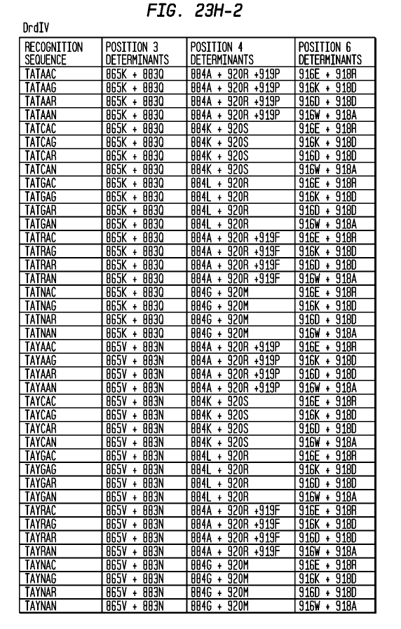

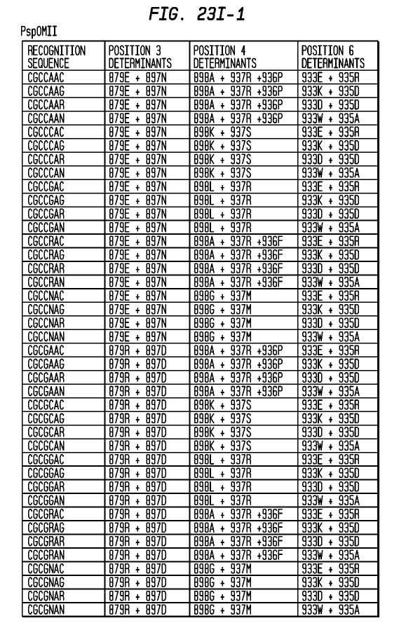

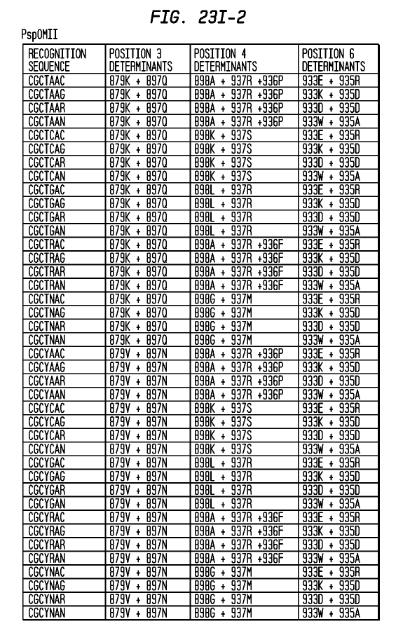

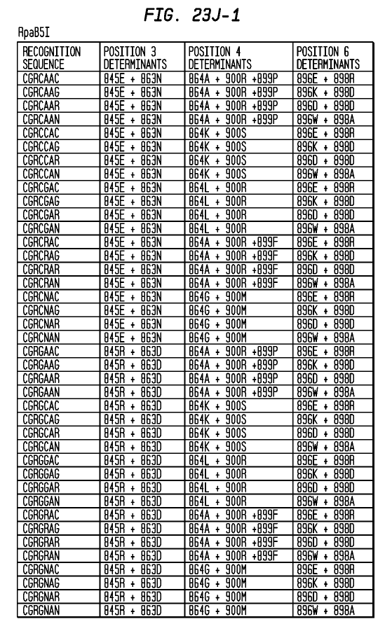

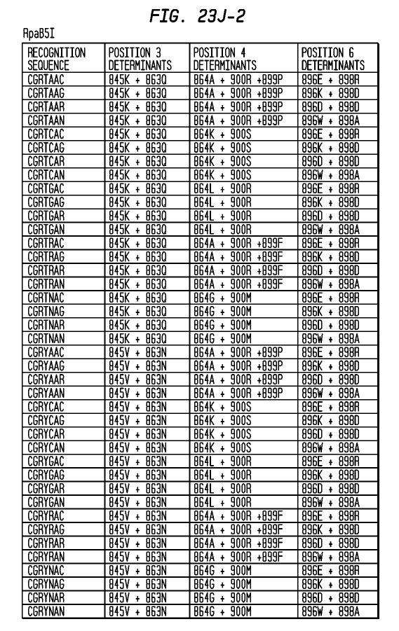

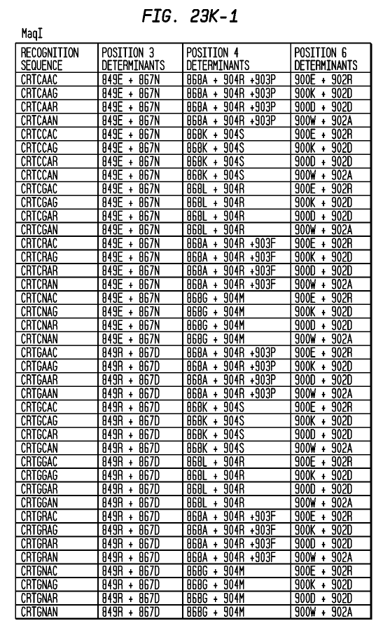

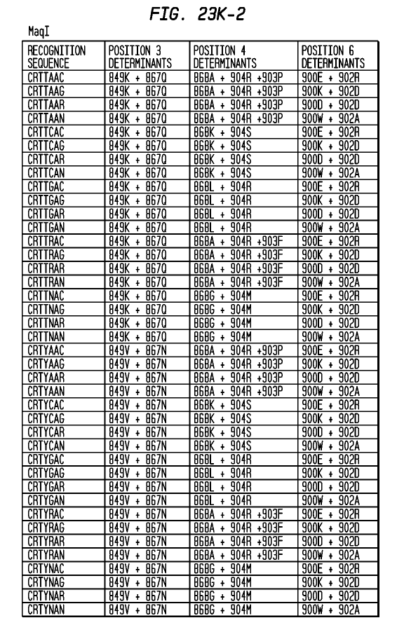

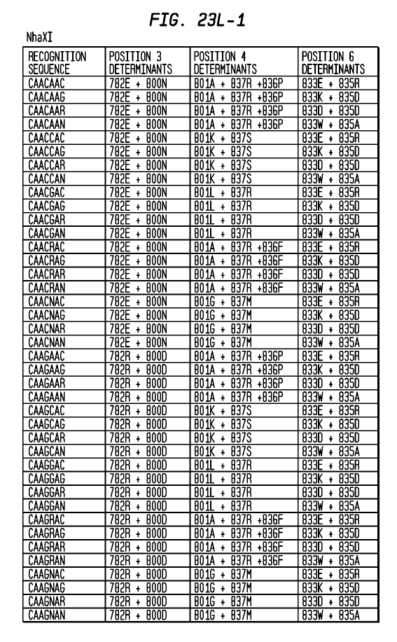

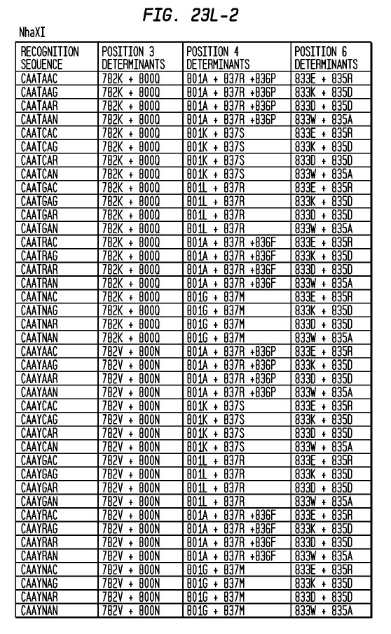

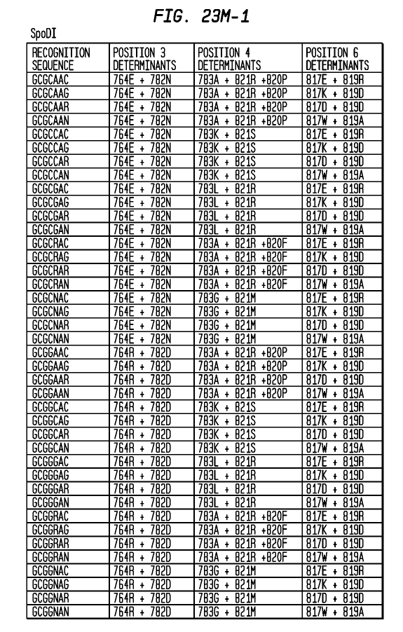

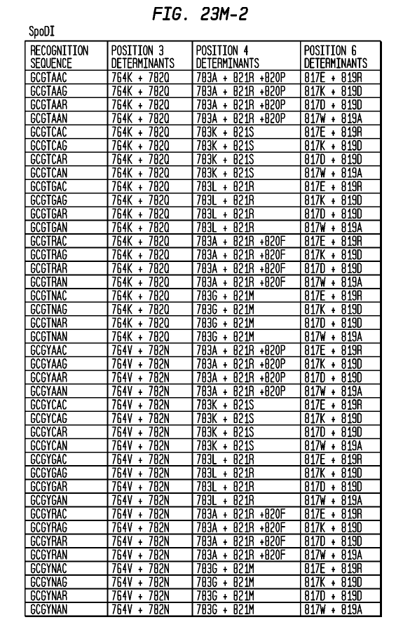

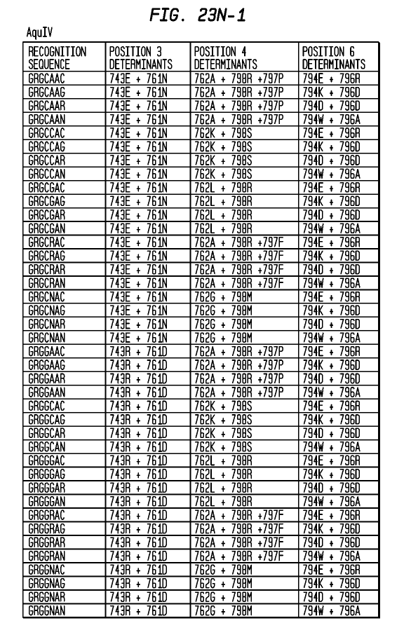

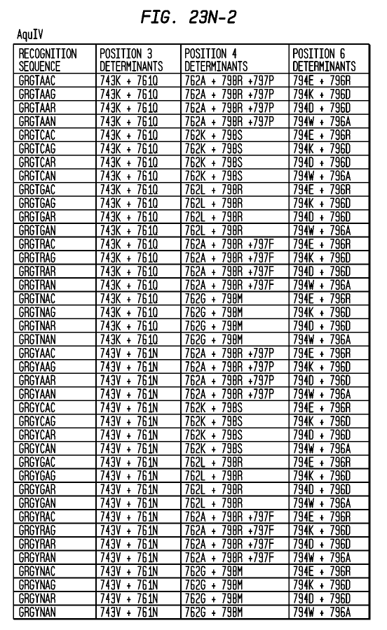

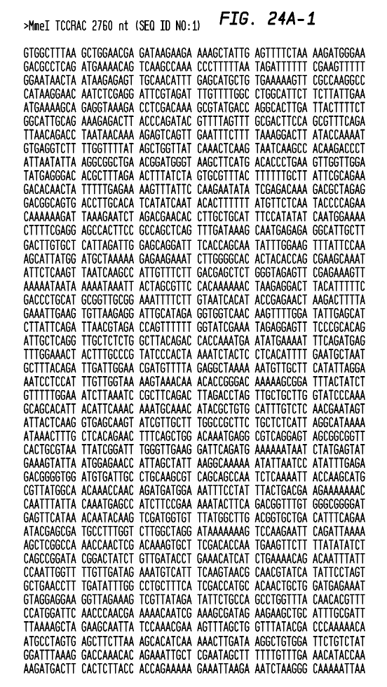

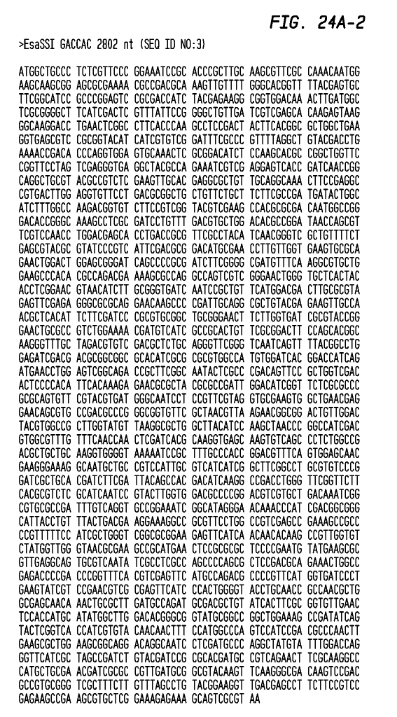

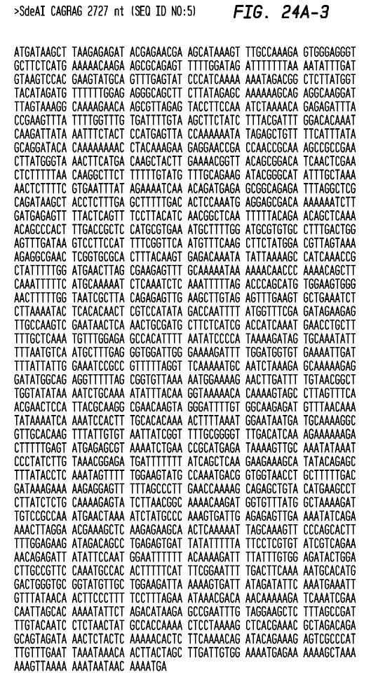

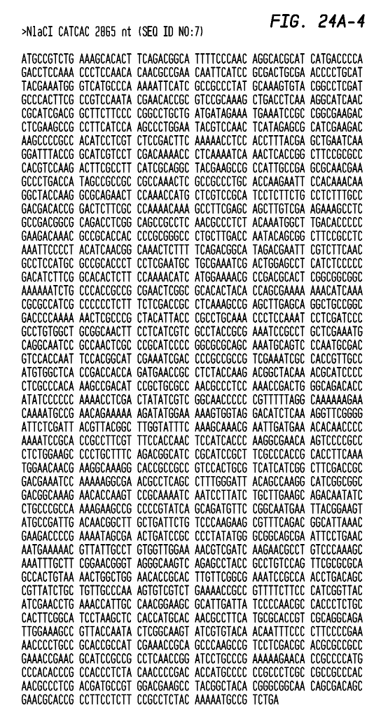

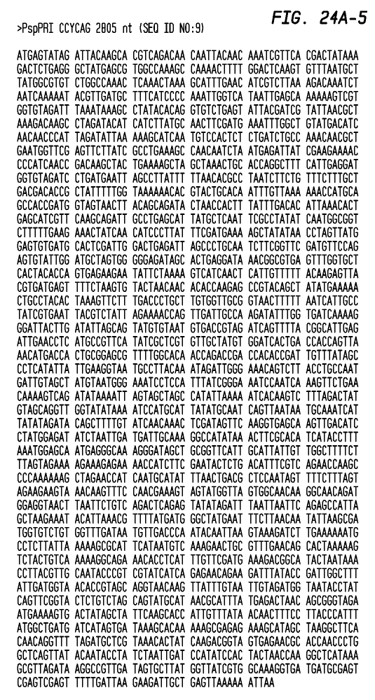

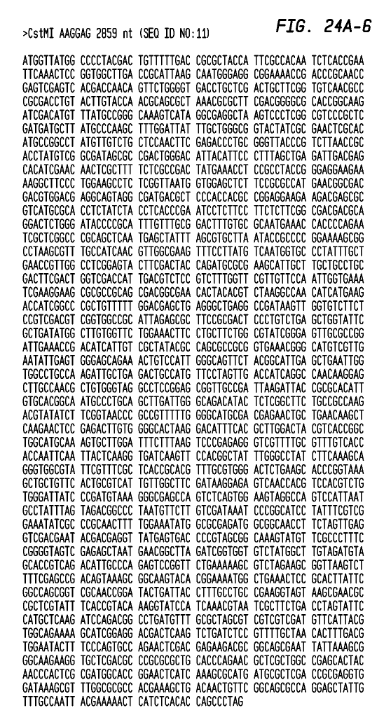

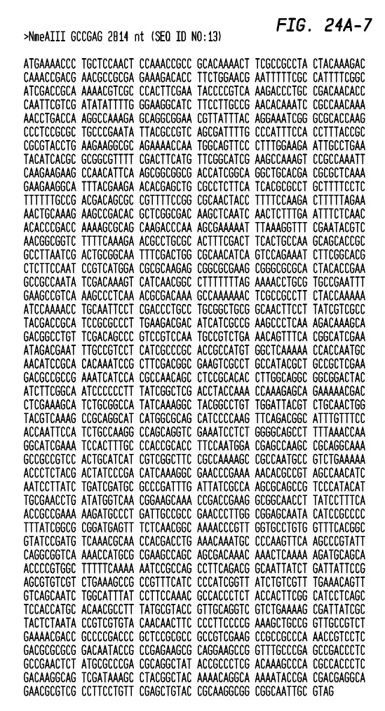

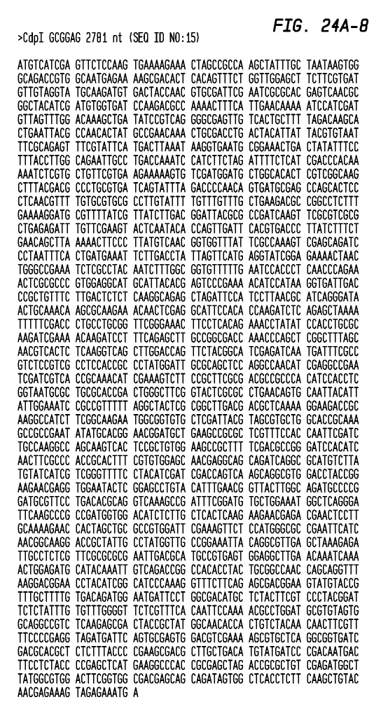

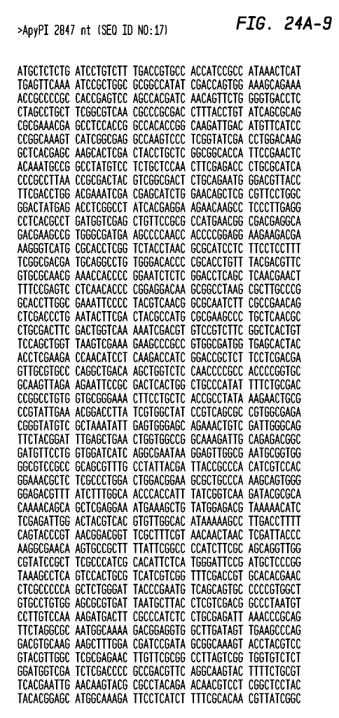

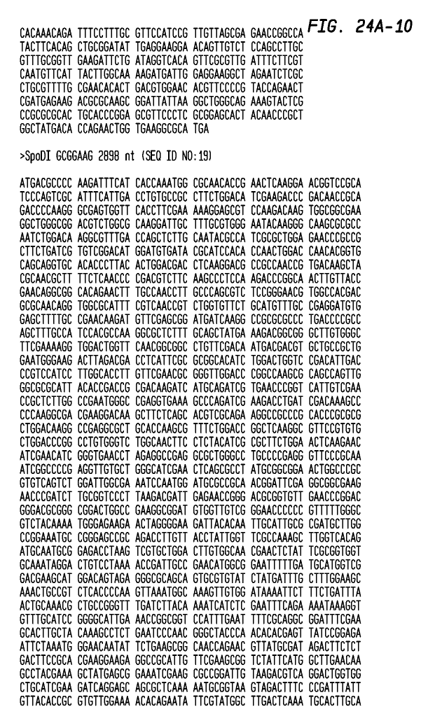

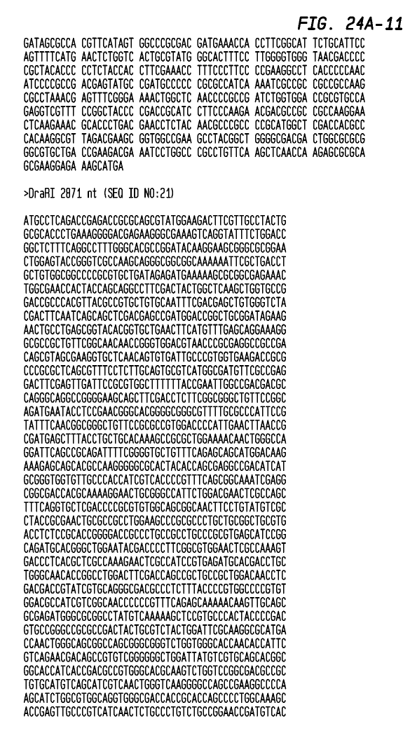

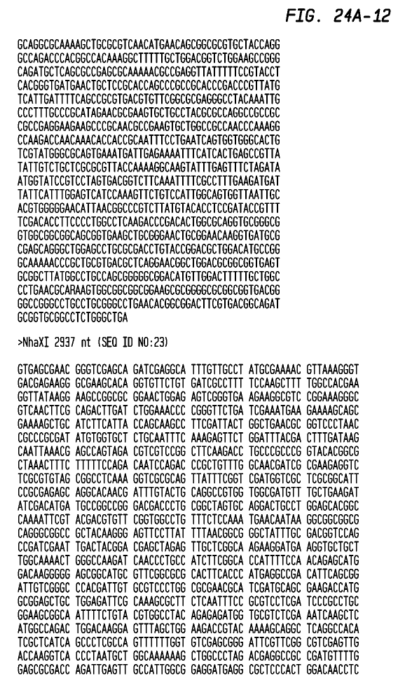

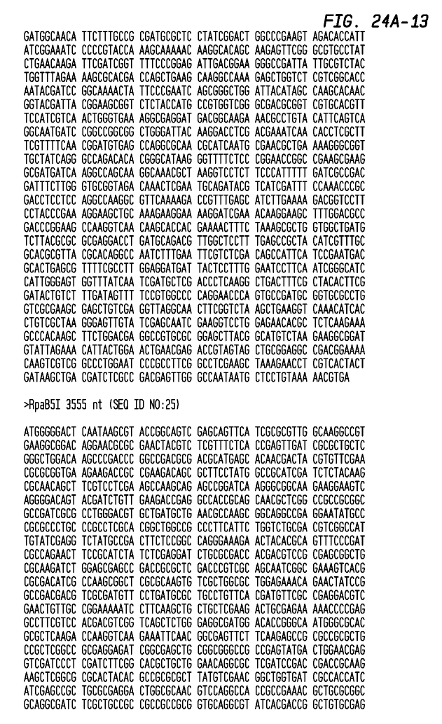

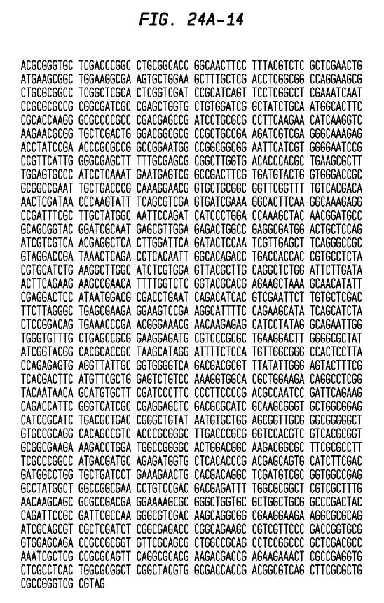

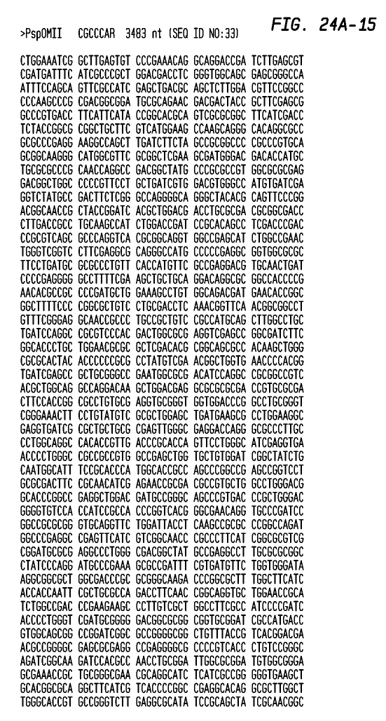

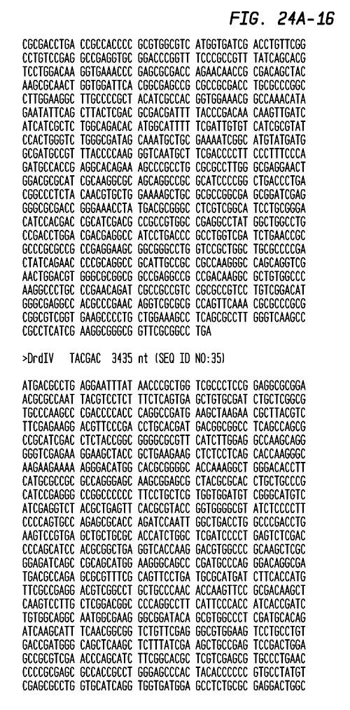

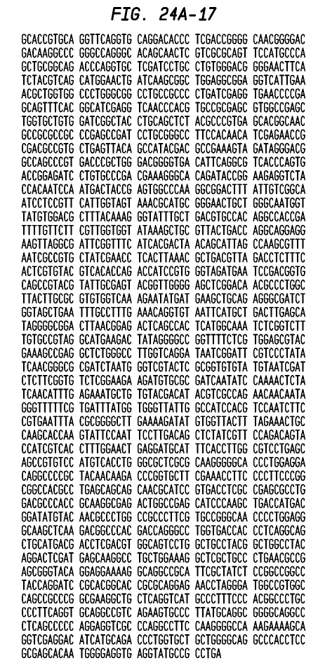

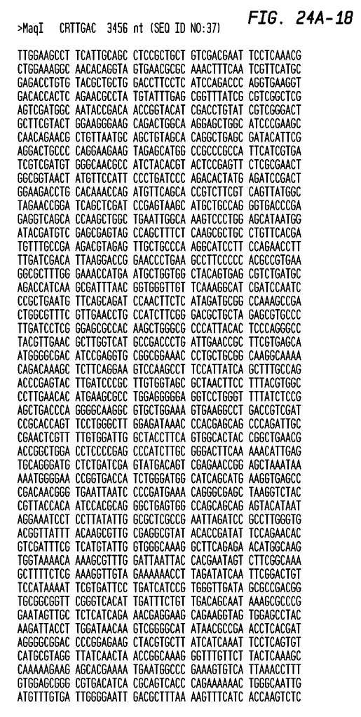

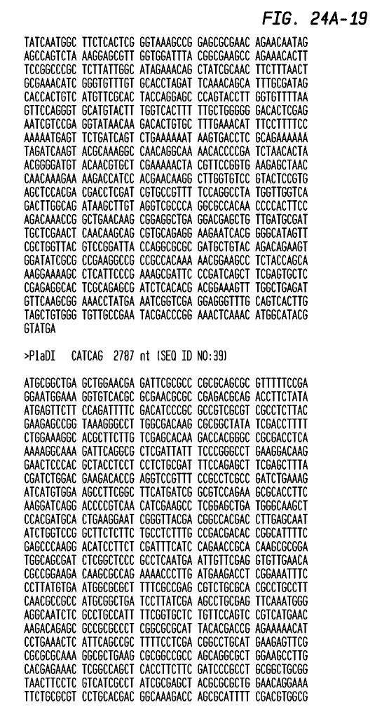

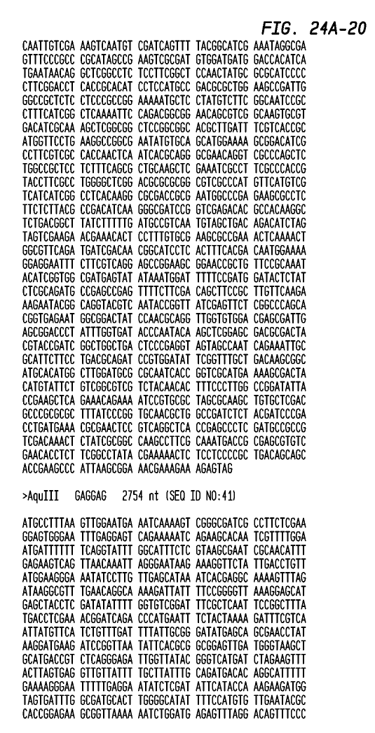

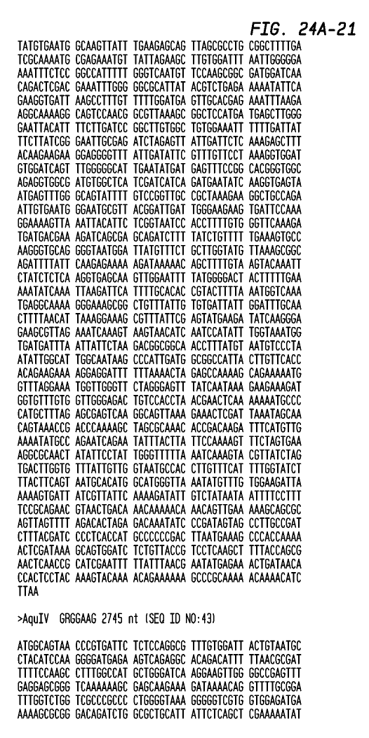

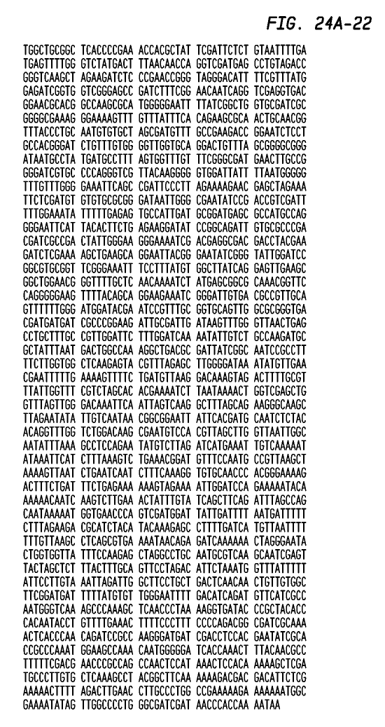

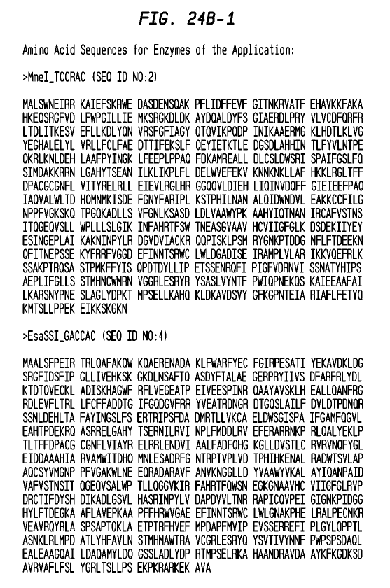

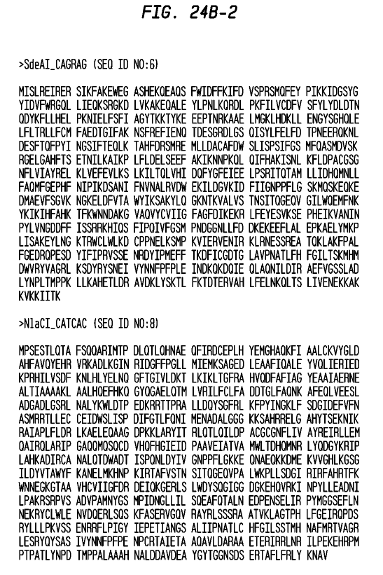

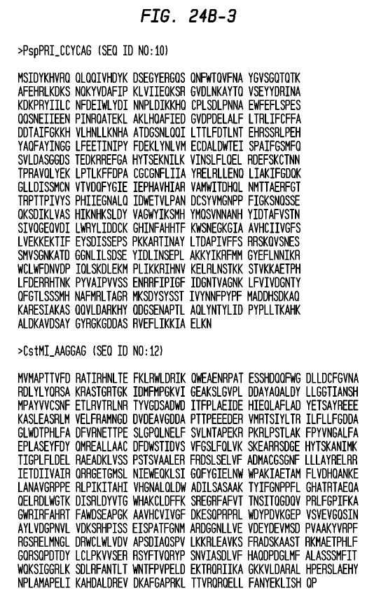

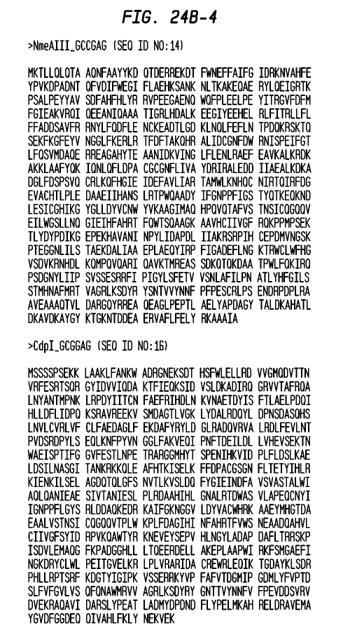

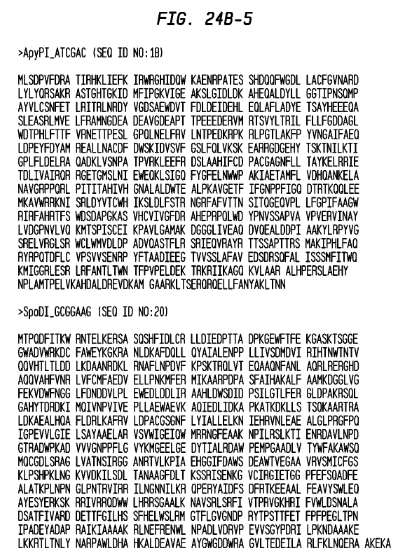

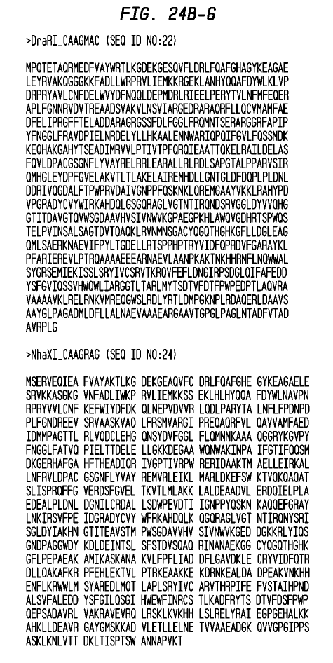

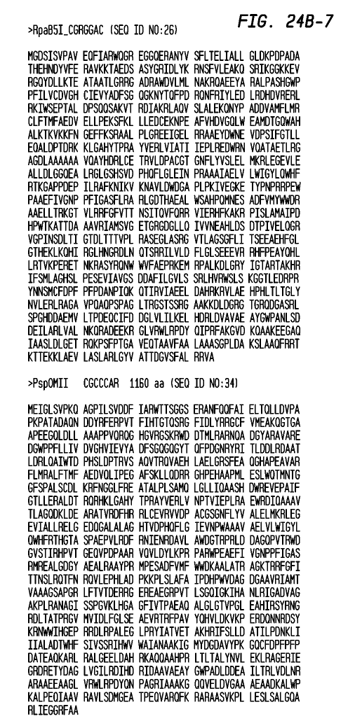

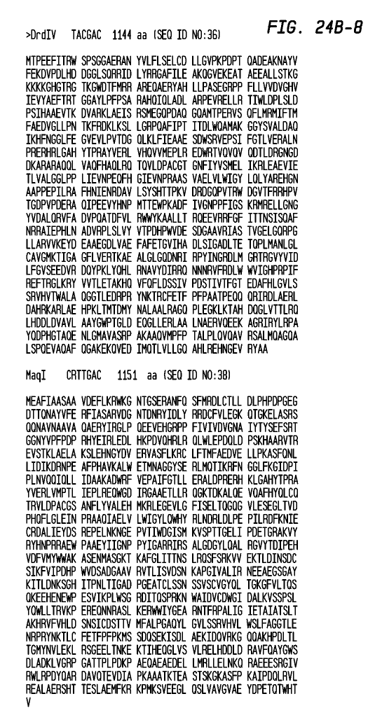

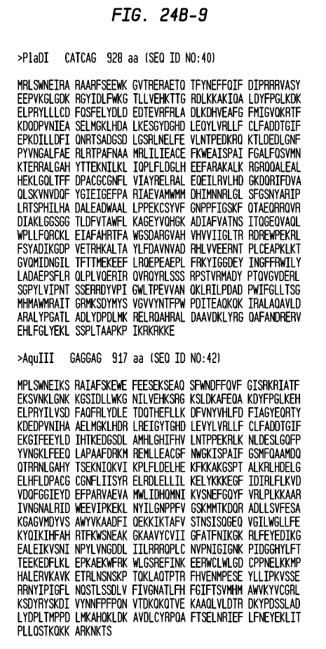

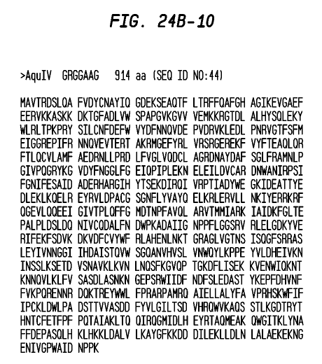

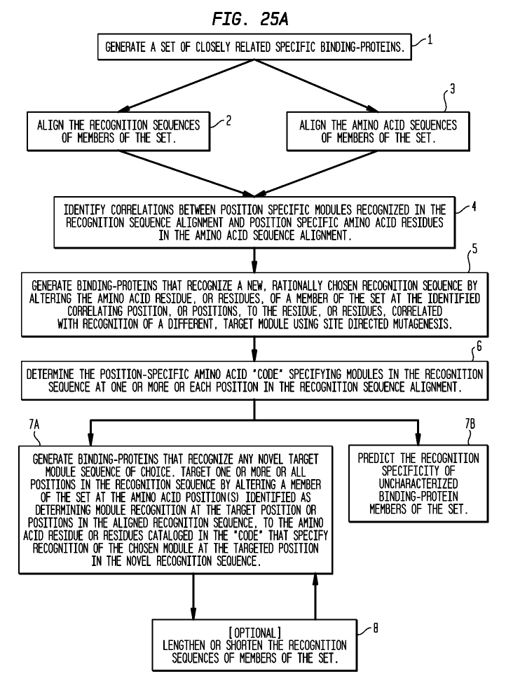

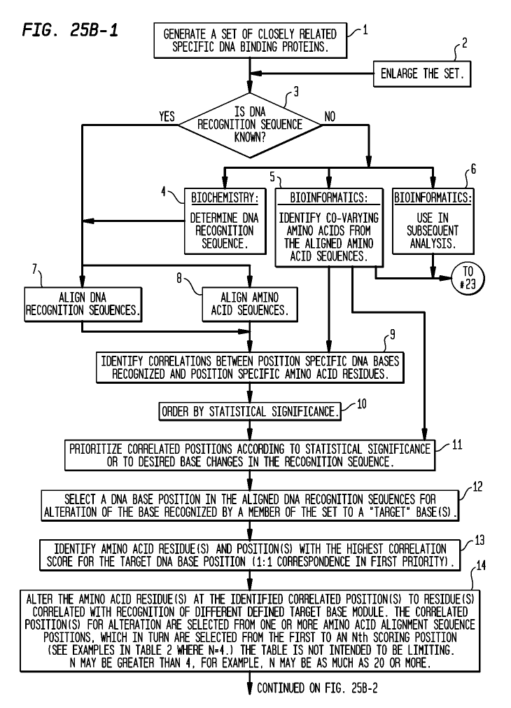

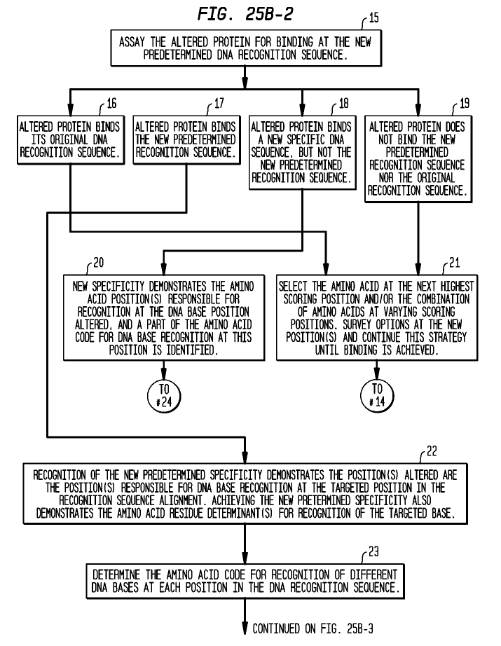

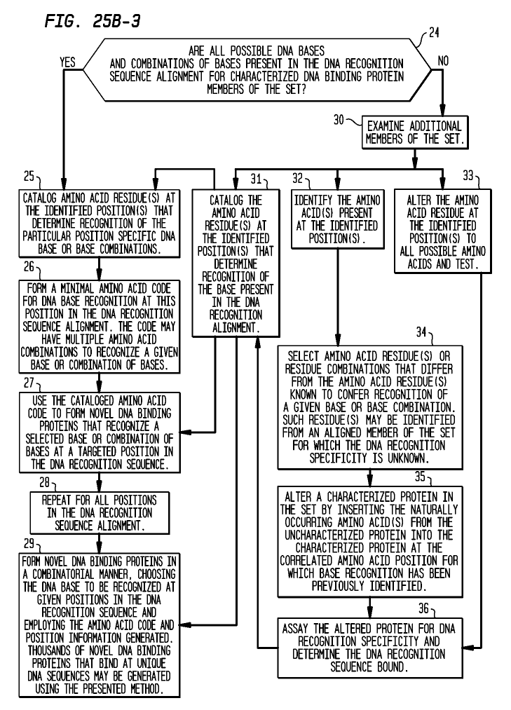

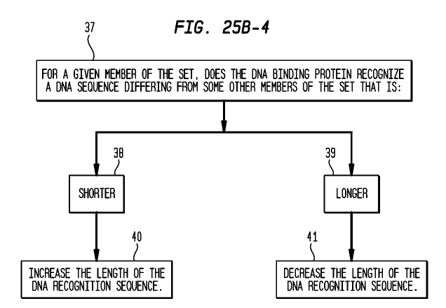

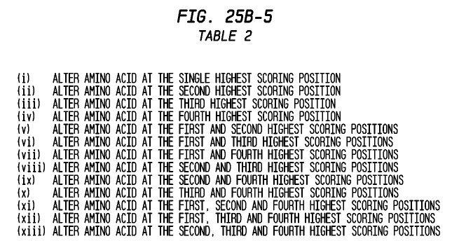

In FIG. 1A, lanes 2-5 show the cleavage pattern produced by the rationally altered MmeI E806K+R808D enzyme on various DNA substrates. The DNA substrate in lane 2 is lambda DNA, in lane 3-T7 DNA, in lane 4-T3 DNA and in lane 5-pBC4 DNA. Lanes 1 and 6 are Lambda-HindIII+PhiX174-HaeIII size standards.

In FIG. 1B, lanes 2-7 show mapping of the cleavage activity of rationally altered MmeI E806K+R808D on pBR322DNA. Lanes 2-7 are pBR322 DNA cut with the rationally altered MmeI E806K+R808D enzyme plus the following single site enzymes: lane 2-EcoRI, lane 3-NruI, lane 4-PvuII, lane 5-NdeI, lane 6-PstI, and lane 7-rationally altered MmeI only. Lanes 1 and 8 are Lambda-HindIII+PhiX174-HaeIII size standards.

In FIG. 1C, the panel shows the location of the wild type MmeI sites, TCCRAC, and of the rationally altered MmeI E806K+R808D sites, TCCRAG, in pBR322DNA, along with the locations of the enzymes used for mapping.

FIG. 2 shows mapping of rationally altered NmeAIII K816E+D818R on pBR322, PhiX and pBC4 DNAs. Lanes 2-5 are pBR322 DNA cut with the rationally altered NmeAIII K816E+D818R enzyme plus the following single site enzymes: lane 2-EcoRI, lane 3-NruI, lane 4-PvuII, and lane 5-PstI. Lanes 7-10 are PhiX174 DNA cut with the rationally altered NmeAIII K816E+D818R enzyme plus the following single site enzymes: lane 7-PstI, lane 8-SspI, lane 9-NciI, and lane 10-StuI. Lanes 12-15 and 17 are pBC4 DNA cut with the rationally altered NmeAIII K816E+D818R enzyme plus the following single site enzymes: lane 12-AvrII, lane 13-PmeI, lane 14-AscI, lane 15-EcoRV, and lane 17-NdeI. Lanes 1, 11 and 16 are Lambda-HindIII+PhiX-HaeIII size standard. Lane 6 is Lambda-BstEII+pBR322-MspI size standard.

FIG. 3 shows the cleavage activity of rationally altered Mme4GI: MmeI A774L.

In FIG. 3A, lanes 2-5 show the cleavage pattern produced by the rationally altered MmeI A774L enzyme on various DNA substrates. Lane 2 is lambda DNA, lane 3-T7 DNA, lane 4-T3 DNA and lane 5-pBR322 DNA. Lanes 7-11 show mapping of the cleavage activity of rationally altered MmeI A774L on PhiX DNA. Lanes 7-11 are PhiX DNA cut with the rationally altered MmeI A774L enzyme plus the following single site enzymes: lane 7-PstI, lane 8-SspI, lane 9-NciI, lane 10-StuI, and lane 11-rationally altered MmeI only. Lanes 1, 6 and 12 are Lambda-HindIII+PhiX174-HaeIII size standards.

In FIG. 3B, lanes 2-8 show mapping of the cleavage activity of rationally altered MmeI A774L on pBC4 DNA. Lanes 2-8 are pBC4 DNA cut with the rationally altered MmeI A774L enzyme plus the following single site enzymes: lane 2-NdeI, lane 3-AvrII, lane 4-PmeI, lane 5-AscI, lane 6-SpeI, lane 7-EcoRV, and lane 8-rationally altered MmeI only. Lanes 1 and 8 are Lambda-HindIII+PhiX174-HaeIII size standards.

FIG. 4 shows the cleavage activity of rationally altered Mme4CI enzyme: MmeI A774K+R801S.

In FIG. 4A, lanes 2-4 show the cleavage pattern produced by the rationally altered MmeI A774K+R801S enzyme on various DNA substrates: lane 2 is lambda DNA, lane 3-T7 DNA and lane 4-T3 DNA. Lanes 1 and 5 are Lambda-HindIII+PhiX174-HaeIII size standards.

FIG. 46 shows mapping of the cleavage activity of rationally altered MmeI A774K+R801S on pBC4 DNA. Lanes 2-8 are pBC4 DNA cut with the rationally altered MmeI A774K+R801S enzyme plus the following single site enzymes: lane 2-NdeI, lane 3-AvrII, lane 4-PmeI, lane 5-AscI, lane 6-SpeI, lane 7-EcoRV, and lane 8-rationally altered MmeI only. Lanes 1 and 8 are Lambda-HindIII+PhiX174-HaeIII size standards.

FIG. 5 shows the cleavage activity of rationally altered Mme3GI enzyme: MmeI E751R+N773D.

FIG. 5A shows mapping of the cleavage activity of rationally altered MmeI E751R+N773D on pUC19 DNA. Lanes 2-6 are pUC19 DNA cut with the rationally altered MmeI E751R+N773D plus the following single site enzymes: lane 2-EcoO109I, lane 3-PstI, lane 4-AlwNI, lane 5-XmnI, and lane 6-MmeI E751R+N773D enzyme alone. Lane 1 is Lambda-HindIII+PhiX-HaeIII size standard. Lane 7 is Lambda-BstEII+pBR322-MspI size standard.

FIG. 5B shows mapping of the cleavage activity of rationally altered MmeI E751R+N773D on pBR322 DNA. Lanes 2-6 are pBR322DNA cut with the rationally altered MmeI E751R+N773D plus the following single site enzymes: lane 2-EcoRI, lane 3-NruI, lane 4-PvuII, lane 5-PstI, and lane 6-MmeI E751R+N773D enzyme alone. Lane 6 is Lambda-HindIII+PhiX-HaeIII size standard. Lane 1 is Lambda-BstEII+pBR322-MspI size standard.

FIG. 5C shows mapping of the cleavage activity of rationally altered MmeI E751R+N773D on PhiX DNA. Lanes 2-6 are PhiX DNA cut with the rationally altered MmeI E751R+N773D plus the following single site enzymes: lane 2-PstI, lane 3-SspI, lane 4-NciI, lane 5-StuI, lane 6-MmeI E751R+N773D enzyme alone. Lane 1 is Lambda-HindIII+PhiX-HaeIII size standard. Lane 7 is Lambda-BstEII+pBR322-MspI size standard.

FIG. 5D shows mapping of the cleavage activity of rationally altered MmeI E751R+N773D on pBC4 DNA. Lanes 2-8 are pBC4 DNA cut with the rationally altered MmeI E751R+N773D enzyme plus the following single site enzymes: lane 2-NdeI, lane 3-AvrII, lane 4-PmeI, lane 5-AscI, lane 6-SpeI, lane 7-EcoRV, and lane 8-rationally altered MmeI only. Lane 1 is Lambda-HindIII+PhiX-HaeIII size standard. Lane 8 is Lambda-BstEII+pBR322-MspI size standard.

FIG. 6 shows the cleavage activity of rationally altered Mme6R1: MmeI E806G+R808G (+S807N).

FIG. 6A shows the cleavage activity of rationally altered MmeI: E806G+R808G (+S807N) on pUC19 DNA. Lanes 2-5 are pUC19 cut with the rationally altered MmeI E806G+R808G (+S807N) plus the following single site enzymes: lane 2-EcoO109I, lane 3-PstI, lane 4-AlwNI, lane 5-XmnI. Lane 1 is Lambda-BstEII+pBR322-MspI size standard. Lane 6 is Lambda-HindIII+PhiX-HaeIII size standard.

FIG. 6B shows the cleavage activity of rationally altered MmeI: E806G+R808G (+S807N) on pBR322 and PhiX174 DNAs. Lanes 2-5 are pBR322 cut with the rationally altered MmeI E806G+R808G (+S807N) plus the following single site enzymes: lane 2-EcoRI, lane 3-NruI, lane 4-PvuII, lane 5-PstI. Lanes 7-10 are PhiX174 cut with the rationally altered MmeI E806G+R808G (+S807N) plus the following single site enzymes: lane 7-PstI, lane 8-SspI, lane 9-NciI, and lane 10-StuI. Lanes 1 and 11 are Lambda-HindIII+PhiX-HaeIII size standard. Lane 7 is Lambda-BstEII+pBR322-MspI size standard.

FIG. 7 shows the cleavage activity of rationally altered Mme6BI enzyme: MmeI E806G+R808T on pUC19, pBR322 and PhiX DNAs. Lanes 2-6 are pUC19 DNA cut with the rationally altered MmeI E806G+R808T enzyme plus the following single site enzymes: lane 2-EcoO109I, lane 3-PstI, lane 4-AlwNI, lane 5-XmnI, and lane 6-MmeI E806G+R808T enzyme alone. Lanes 8-12 are pBR322DNA cut with the rationally altered MmeI E806G+R808T enzyme plus the following single site enzymes: lane 8-ClaI, lane 9-NruI, lane 10-NdeI, lane 11-PstI, and lane 12-MmeI E806G+R808T enzyme alone. Lanes 14-18 are PhiX DNA cut with the rationally altered MmeI E806G+R808T enzyme plus the following single site enzymes: lane 14-PstI, lane 15-SspI, lane 16-NciI, lane 17-StuI, and lane 18-MmeI E806G+R808T enzyme alone. Lanes 1 and 13 are Lambda-HindIII+PhiX-HaeIII size standard. Lanes 7 and 19 are Lambda-BstEII+pBR322-MspI size standard.

FIG. 8 shows the cleavage activity of rationally altered Mme6NI enzyme: MmeI E806W+R808A on phage φX DNA. Lanes 2-4 and 6-8 are phage φX DNA cut with the rationally altered MmeI E806W+R808A enzyme plus the following single site enzymes: lane 2-PstI, Lane 3-SspI, lane 4-NciI, lane 6-StuI, lane 7-BsiEI, and lane 8-MmeI E806W+R808A enzyme alone. Lanes 1 and 9 are Lambda-HindIII+PhiX-HaeIII size standard. Lane 5 is Lambda-BstEII+pBR322-MspI size standard.

FIG. 9 shows the cleavage activity of rationally altered SdeA6CI enzyme: SdeAI K791E+D793R on pUC19, pBR322 and PhiX DNAs. Lanes 2-6 are pUC19 DNA cut with the rationally altered SdeAI K791E+D793R enzyme plus the following single site enzymes: lane 2-EcoO109I, lane 3-PstI, lane 4-AlwNI, lane 5-XmnI, and lane 6-SdeAI K791E+D793R enzyme alone. Lanes 8-12 are pBR322DNA cut with the rationally altered SdeAI K791E+D793R enzyme plus the following single site enzymes: lane 8-EcoRI, lane 9-NruI, lane 10-PvuII, lane 11-PstI, and lane 12-SdeAI K791E+D793R enzyme alone. Lanes 14-18 are PhiX DNA cut with the rationally altered SdeAI K791E+D793R enzyme plus the following single site enzymes: lane 14-PstI, lane 15-SspI, lane 16-NciI, lane 17-StuI, and lane 18-SdeAI K791E+D793R enzyme alone. Lanes 1, 13 and 20 are Lambda-HindIII+PhiX-HaeIII size standard. Lanes 7 and 19 are Lambda-BstEII+pBR322-MspI size standard.

FIG. 10 shows DNA bases observed at each position in the recognition sequence alignment for the characterized members of the set.

FIG. 10A shows in the left panel the DNA recognition sequence alignment of the characterized members of the set containing MmeI as a member (the MmeI-like set). These recognition sequences include BsbI enzyme, for which the DNA recognition sequence and cutting positions are known, but for which the amino acid sequence has not yet been determined. The right panel shows the count for the various DNA bases, or combination of bases, recognized at each position in the DNA recognition sequence alignment.

FIG. 10B shows in the left panel the alignment of the recognition sequence of 20 members of the MmeI-like set. The right panel is a position-defined base frequency chart showing the DNA bases observed at position 3, 4 or 6 in the recognition sequence alignment for the characterized members of the set. Nineteen of twenty enzymes recognize G or C at the sixth position.

FIG. 11A shows a partial code for the amino acids correlated with DNA base recognition at position 3, position 4 or position 6 in the recognition sequence alignment. For example, to alter recognition at position 6 of the aligned recognition sequences in a member of the set, the positions in the amino acid sequence alignment corresponding to MmeI E806 and R808 are the targets for mutating the amino acid to one of the coded alternative amino acid residues to redesign DNA base recognition. For example, inserting the code E+R into a member of the MmeI-like set at these aligned positions would cause the enzyme to recognize a C base at position 6 of that enzyme's recognition sequence. The code can be expanded as the members of the set increase, and their amino acid substitutions are tested for changes in DNA recognition sequence specificities.

FIG. 11B shows the identified positions within the aligned amino acid sequences (SEQ ID NOS:64-82), and the amino acid residues occupying those positions, that determine recognition at position 3, 4 or 6 in the aligned DNA recognition sequences. The number above the alignment indicates the position in the recognition sequence for which that amino acid position determines the DNA base recognized. The enzyme name and the DNA sequence recognized is shown. The number preceding the aligned amino acid sequence indicates the position of the first amino acid residue listed within the amino acid sequence of the enzyme, while the number following the line of amino acid sequence indicates the position of the last amino acid residue listed in the sequence of the enzyme.

FIG. 12 shows an amino acid sequence alignment of SEQ ID NOS:100-131 (an MmeI-like set) in which amino acid residues are identified, at positions characterized as determining recognition at position 6 in the recognition sequence, that differ from known DNA base recognition determinants. Members of the set for which the DNA recognition sequence has not yet been characterized have been included in this alignment. The two arrows indicate the positions identified that determine recognition of the DNA base at position 6 (position 1073 and 1077 in this gapped CLUSTALW alignment). There are four sequences, which are underlined, in which the amino acid residue pairs observed do not match the pairs present in any previously characterized member of the set. These position-specific pairs are naturally occurring variations that are targets for introduction into a characterized enzyme as a means of altering the specificity of the characterized enzyme at the targeted DNA base recognition position. Two of the observed differing pairs, GXS (two occurrences) and G(N)G were introduced into the characterized enzyme MmeI and the DNA recognition specificity of the resulting rationally altered enzyme was investigated (see FIG. 6)

FIG. 13 shows the prioritization of correlated positions for alteration. The first priority for alteration to change the specificity of a member of the set are those positions that exhibit a 1:1 correlation between the amino acid residue present at that position in the alignment and the DNA base recognized at the position in the recognition sequence alignment being interrogated.

The top panel shows the amino acid sequence alignment of SEQ ID NOS:132-150) that is ordered with respect to position 6 of the recognition sequence alignment, in which the residues at the aligned position encompassing MmeI R808 (indicated by the arrow) are correlated one to one with the DNA base recognized at position 6. At this position all enzymes that recognize C, cytosine, have an arginine residue, R, and all enzymes that recognize a G, guanine, have an aspartate residue, D.

The lower panel has two arrows, one to identify the 1:1 correlating position described above, and the second to indicate the second highest scoring position. This second position, while not correlating 1:1, is still statistically significantly correlated with recognition of the DNA base at position 6, as exemplified in FIG. 14. In addition, the amino acid residue at this position co-varies with the residue at the 1:1 correlating position described above in 7 of 8 enzymes that recognize C and 9 of 10 enzymes that recognize G, indicating this position is likely to be partnering with the 1:1 correlating position to recognize the base position in question. This position becomes the second highest priority for change, and may be rationally altered together with the first highest priority position to effect the desired alteration in DNA recognition specificity.

FIG. 14 shows a Chi square calculation for one position in the amino acid alignment that correlates with recognition of the base at position 6 of the aligned recognition sequences. For the Chi square calculation a table is formed consisting of a row for each different DNA base recognized at the position in the recognition sequence alignment under investigation, and a column for each amino acid residue present at the given position in the amino acid sequence alignment. Here such a table consists of three rows, one each for the DNA base patterns, C, G and R, recognized at position 6 of the recognition sequence alignment, and of five columns, one each for the amino acid residues present at the position interrogated in the amino acid sequence alignment. The position interrogated is that which aligns with MmeI position E806. The count of the amino acid residues present at this position is shown. The calculated Chi square value for the table is 38. There are 8 degrees of freedom in the table. The resulting probability value, P, is 0.0001, which is less than the cut off for significance of 0.05. The result indicates this amino acid position is significantly correlated with recognition of the DNA base at position 6 of the DNA recognition sequence alignment.

FIG. 15 shows correlations between aligned DNA recognition sequences at position 6 and two positions in the amino acid sequence alignment.

In the left panel, the aligned DNA recognition sites are grouped into the 9 enzymes, which have a C at position 6, followed by the 10 enzymes, which have a G at this position, followed by the one enzyme that has an R at this position.

In the right panel, a portion of the amino acid sequence for nineteen enzymes from the MmeI-like set is aligned to reveal a region where a correlation is observed between the DNA base recognized at position 6 and the amino acid residue(s) present in the aligned protein sequences. Arrows indicate the two correlating amino acid positions identified. They correspond to E806 and R808 of MmeI. At position R808 of the gapped alignment shown there is a 1:1 correspondence between the amino acid and the DNA base recognized in position 6, such that whenever an enzyme recognizes a C base there is an arginine, R, at this position, while those enzymes recognizing a G base have an aspartic acid residue, D, at this position. The enzyme recognizing R, which is G or A, also has an aspartate, D, at this position. The E806 position does not have complete 1:1 correspondence, due to the biological flexibility allowing more than one amino acid residue to partner with either the arginine of position R808 to recognize a C base, in this case either E, glutamic acid or T, threonine, or with the aspartic acid residue of position R808 to recognize a G base, here either a K, lysine or a G, glycine, or with the arginine of position R808 to recognize R (A or G), which here is a D residue. There is also a three amino acid residue insertion just preceding this aspartic acid residue in the enzyme recognizing R, PspOMII.

FIGS. 16-1, 16-2 and 16-3 show that the set of sequences may be enlarged through a BLAST search initiated from previously identified members of the set. Here, the SpoDI amino acid sequence was used as the query.

The results of a BLAST search demonstrate that a member of the set of related proteins identified through the initial BLAST search can be used as the query sequence for a subsequent BLAST search. In this case a sequence identified in a BLAST search starting with MmeI as the query, ref|YP_{—}167160.1 “hypothetical protein SPO1926,” was used as the query to perform a subsequent BLAST search. The default parameters of the blastp program at the ncbi BLAST server were used: http://www.ncbi.nlm.nih.gov/BLAST/. Use of a different member of the set as the BLAST query resulted in identification of several additional members of the set. For example, the ref|YP_{—}511167.1 “hypothetical protein Jann_{—}3225” sequence was excluded from the set by the stringent threshold of E<e-20 when the search was initiated using the MmeI sequence (E=5e-17, FIGS. 18-1, 18-2 and 18-3), but this Jann_{—}3225 sequence is shown to be a member of the set when the BLAST search is made using as query the “SPO1926” member of the set, for in this case the Expectation value returned is E=3e-65. The set may be enlarged by searches in which the various members of the set serve as the query sequence. Because the Expectation value cut off is stringent, the set will not be enlarged unendingly, but will merely expand to encompass more members of the related set than may be found by searching from a single starting sequence.

FIG. 17 shows a DNA base recognition table listing the 15 different DNA bases or combinations of DNA bases that may be recognized at any given position within a DNA recognition sequence.

FIGS. 18-1, 18-2 and 18-3 show the BLAST search results identifying a set of sequences highly similar to MmeI when the MmeI amino acid sequence was used a the query.

The default parameters of the blastp program at the ncbi BLAST server http)://www.ncbi.nlm.nih.gov/BLAST/. Ninety-seven protein sequences are identified that have Expectation Values, E, of E<e-20. One such sequence, ref|YP_{—}167160.1 “hypothetical protein SPO1926,” returns an E value in this search of E=6e-47. As an example, this member of the set may be used in a subsequent BLAST search to enlarge the set of related proteins. Such a search may enlarge the set by identifying proteins that are related to the family as a whole, but which happen to be just distant enough from the sequence used for the first BLAST search that they return Expectation values just outside of the cut off threshold in the initial search. Such a sequence, ref|YP_{—}511167.1 “hypothetical protein Jann_{—}3225,” that falls just outside of the cut off threshold in the search using the MmeI amino acid sequence, but that is included in the set (FIGS. 16-1, 16-2 an 16-3) when enlarged by a search using a different member of the set, the “SPO1926” sequence, is underlined.

FIG. 19 shows the alignment of DNA recognition sequences recognized by 20 characterized members of the MmeI-like set of related DNA binding proteins. The alignment was made in relation to a common function. The single strand chosen for alignment from the double stranded DNA that is recognized by the enzyme is the strand that is cut 3′ to the recognition sequence. The alignment is then anchored about the common adenine base at position 5 that is functionally conserved, in that it is the base modified by the methyltransferase activity of the enzymes.

FIGS. 20-1 to 20-11 show an amino acid sequence alignment of SEQ ID NOS:42, 6, 10, 4, 2, 40, 8, 14, 18, 12, 16, 26, 34, 38, 36, 20, 44, 24, and 22, formed using the algorithm PROMALS, for 19 characterized members of the set of related DNA binding proteins whose recognition sequences are shown in FIG. 19.

FIG. 21 shows a Chi square calculation for aligned positions in an amino acid sequence alignment. Chi square value is the sum for all observations (positions in the table) of the: ((observed frequency minus the expected frequency) squared) divided by the expected frequency). A contingency table is constructed where one row is utilized for each DNA base recognized at the position within the DNA recognition sequence alignment being interrogated. The rows are the DNA base observed (Bobs1) through as many different DNA bases as are observed at the position in the recognition sequence alignment being examined. One column is utilized for each amino acid residue observed at the given position in the amino acid sequence alignment being examined. The columns are labeled from the first amino acid residue observed (AA-obs1) through as many different amino acid residues observed at the aligned position.

The observed frequency is the count of amino acid residues at the aligned position for the DNA base recognized. The expected frequency is the sum of the column in which the observation occurs times the sum of the row in which the observation occurs, divided by the total count of all observations.

The table is then populated with the observed counts for the amino acid residues present at the given position in the amino acid sequence alignment, placing the amino acid residue counts within their particular columns in the row corresponding to the DNA base recognized by the binding protein in which that amino acid residue occurs.

The Chi square value for the observed counts is calculated from the table. The statistical significance (P-value) of the Chi square value is obtained by comparing the Chi square value to a Chi square statistics table, where the degrees of freedom equal [(the number of columns minus one) times (the number of rows minus 1)]. If the P-value is less than the preset threshold (0.05 is the default), the algorithm reports this amino acid alignment position as significantly correlated to the interrogated position of the DNA recognition sequence.

The analysis is repeated for each position in the DNA recognition alignment together with each position in the amino acid recognition alignment.

FIG. 22 shows identification of a position in an amino acid sequence alignment, and the specific amino acids at that position, that participates in recognition of the third position in the aligned DNA recognition sequences of a set of gamma-class N6A DNA methyltransferases. The figure shows an alignment of the DNA recognition sequences of the members of the set, anchored about the adenine target of methylation at position 5. A portion of the aligned amino acid sequences of the proteins is shown (SEQ ID NOS:83-99). The particular amino acid coordinates for each protein are indicated before and following the sequence for each enzyme. A position in the alignment that correlates significantly with the DNA base recognized by the enzymes at position 3 is indicated by a box and labeled with a “3” above the alignment.

FIGS. 23A-23N show a partial list of enzymes having differing DNA recognition sequences. The position-specific amino acids required to generate these enzymes within the sequence context of the starting enzyme are listed for each recognition sequence. Specifically, the positions within the amino acid sequence of the starting protein and the amino acids required at those positions for recognition of the listed DNA recognition sequence are described. To create using chemistry any of the specificities provided in the left column, the columns to the right are consulted and, if an alteration in the amino acid at the listed position is required, this is introduced by rationally altering the starting protein listed at the top of the figure at the specified position. FIGS. 23A-23N provide starting enzymes having the listed recognition sequences: MmeI (SEQ ID NO: 2), NmeAIII (SEQ ID NO: 14), SdeAI (SEQ ID NO: 6), CstMI (SEQ ID NO: 12), ApyPI (SEQ ID NO: 18), PspRI (SEQ ID NO: 10), AquIII, (SEQ ID NO: 42), DrdIV (SEQ ID NO: 36), PspOMII (SEQ ID NO: 34) RpaB5I (SEQ ID NO: 26), MaqI (SEQ ID NO: 38), NhaXI (SEQ ID NO: 24), SpoDI (SEQ ID NO: 20) and AquIV (SEQ ID NO: 44). These enzymes may be modified at the specified positions by a targeted mutation to provide the desired amino acid residues at the specified positions to generate an enzyme recognizing the listed DNA sequence.

FIGS. 24A-1 to 24A-22 and 24B-1 to 24B-10 contain the DNA sequences (SEQ ID NOS:1, 3, 5, 7, 9, 11, 13, 15, 17, 19, 21, 23, 25, 33, 35, 37, 39, 41 and 43) and corresponding amino acid sequences (2, 4, 6, 8, 10, 12, 14, 16, 18, 20, 22, 24, 26, 34, 36, 38, 40, 42 and 44) for the 19 characterized proteins in the MmeI-like set in FIGS. 20-1 to 20-11.

FIGS. 25A and 25B-1 to 25B-5 show a summary flow diagram and a detailed example describing the methods.

FIG. 25A describes the generation of a set of closely related specific binding proteins capable of recognizing localized position-specific defined modules in a specific substrate (recognition sequence) (1) where the module recognition sequences of members of the set are aligned (2) and the amino acid sequences of the members of the set are separately aligned (3). Correlations are identified between position-specific modules in the recognition sequence alignment and position-specific amino acid residues in the amino acid sequence alignment (4). Binding proteins are generated that recognize new rationally chosen module sequences by altering amino acid residue(s) of a member of the set at the identified correlating position(s) to the residue(s) correlated with recognition of a different target module using site-directed mutagenesis (5). The ability to create a specific amino acid “code” specifying a particular module recognition at one or more or each position in the recognition alignment is thus improved using the steps of 1-5 (6). Binding proteins are generated with a novel recognition sequence by determining the position of the module in a recognition sequence to be rationally altered. The amino acid(s) in the binding protein correlated with the binding specificity for that position-specific module is rationally altered according to amino acid residue(s) in the cataloged code (7A). Alternatively, the module recognition specificity of uncharacterized or new binding protein members of a set can be predicted using the cataloged code (7B). Optionally, additionally, the recognition sequences can be lengthened or shortened for members of the set of binding proteins (8).

FIGS. 25B-1 to 25B-4 show a multi-step approach to analyzing correlations between amino acid sequences in binding proteins that bind position-specific modules in specific recognition sequences to which the binding protein binds. In this Figure, the method is illustrated by means of a DNA binding protein but the method can be equally applied to any binding protein that recognizes a substrate defined by position specific modules in a specific recognition sequence. The information obtained in steps 1-23 is stored as a cataloged code and used to rationally design novel binding proteins (steps 24-30) or to characterize specific recognition sequences for binding proteins whose amino acid sequence already exists in sequence databases (steps 24-37). In addition, steps are provided to generate binding proteins with increased or decreased base pairs in the DNA recognition sequence (steps 38-41).

The text in the numbered boxes is as follows:
1. Generate a set of closely related specific DNA binding proteins. 2. Enlarge the set, 3. Is DNA recognition sequence known?4. Biochemistry: Determine DNA recognition sequence. 5. Bioinformatics: Identify co-varying amino acids from the aligned amino acid sequences. 6. Bioinformatics: Use in subsequent analysis. 7. Align DNA recognition sequences. 8. Align amino acid sequences. 9. Identify correlations between position specific DNA bases recognized and position specific amino acid residues. 10. Order by statistical significance. 11. Prioritize correlated positions according to statistical significance or to desired base changes in the recognition sequence. 12. Select a DNA base position in the aligned DNA recognition sequences for alteration of the base recognized by a member of the set to a “target” base(s). 13. Identify amino acid residue(s) and position(s) with the highest correlation score for the target DNA base position (1:1 correspondence in first priority). 14. Alter the amino acid residue(s) at the identified correlated position(s) to residue(s) correlated with recognition of a different defined target base module. The correlated position(s) for alteration are selected from one or more amino acid alignment sequence positions, which in turn are selected from the first to an Nth scoring position (see examples in Table 1 where N=4.) The Table is not intended to be limiting. N may be greater than 4, for example, N may be as much as 20 or more.). 15. Assay the rationally altered protein for binding at the new predetermined DNA recognition sequence. 16. Rationally altered protein binds its original DNA recognition sequence. 17. Altered protein binds the new predetermined recognition sequence. 18. Altered protein binds a new specific DNA sequence, but not the new predetermined recognition sequence. 19. Altered protein does not bind the new predetermined recognition sequence nor the original recognition sequence. 20. New specificity demonstrates the amino acid position(s) responsible for recognition at the DNA base position altered, and a part of the amino acid code for DNA base recognition at this position is identified. 21. Select the amino acid at the next highest scoring position and/or the combination of amino acids at varying scoring positions. Survey options at the new position(s) and continue this strategy until binding is achieved. 22. Recognition of the new predetermined specificity demonstrates the position(s) altered are the position(s) responsible for DNA base recognition at the targeted position in the recognition sequence alignment. Achieving the new predetermined specificity also demonstrates the amino acid residue determinant(s) for recognition of the targeted base. 23. Determine the amino acid code for recognition of different DNA bases at each position in the DNA recognition sequence. 24. Are all possible DNA bases and combinations of bases present in the DNA recognition sequence alignment for characterized DNA binding protein members of the set? 25. Catalog amino acid residue(s) at the identified position(s) that determine recognition of the particular position specific DNA base or base combinations. 26. Form a minimal amino acid code for DNA base recognition at this position in the DNA recognition sequence alignment. The code may have multiple amino acid combinations to recognize a given base or combination of bases. 27. Use the cataloged amino acid code to form novel DNA binding proteins that recognize a selected base or combination of bases at a targeted position in the DNA recognition sequence. 28. Repeat for all positions in the DNA recognition sequence alignment. 29. Form novel DNA binding proteins in a combinatorial manner, choosing the DNA base to be recognized at given positions in the DNA recognition sequence and employing the amino acid code and position information generated. Thousands of novel DNA binding proteins that bind at unique DNA sequences may be generated using the presented method. 30. Examine additional members of the set. 31. Catalog the amino acid residue(s) at the identified position(s) that determine recognition of the base present in the DNA recognition alignment. 32. Identify the amino acid(s) present at the identified position(s). 33. Alter the amino acid residue at the identified position(s) to all possible amino acids and test. 34. Select amino acid residue(s) or residue combinations that differ from the amino acid residue(s) known to confer recognition of a given base or base combination. Such residue(s) may be identified from an aligned member of the set for which the DNA recognition specificity is unknown. 35. Alter a characterized protein in the set by inserting the naturally occurring amino acid(s) from the uncharacterized protein into the characterized protein at the correlated amino acid position for which base recognition has been previously identified. 36. Assay the altered protein for DNA recognition specificity and determine the DNA recognition sequence bound. 37. For a given member of the set, does the DNA binding protein recognize a DNA sequence differing from some other members of the set that is: 38. Shorter, 39. Longer?40. Increase the length of the DNA recognition sequence. 41. Decrease the length of the DNA recognition sequence

FIG. 25B-5 shows a scheme for prioritizing the amino acid position or positions at which to alter the amino acid residue or residues to residues correlated with recognition of a differing module in the recognition sequence alignment in order to determine the positions that determine recognition of the module at the position in the recognition sequence being investigated. The position in the amino acid sequence alignment that produces the highest correlation score, i.e., the lowest P value, is the first position to test, followed by the second highest correlation scoring position, etc. Since recognition of a module may require more than one amino acid residue in the protein, the two positions having the highest correlation score are the first priority for alteration of two residues together. If alteration at the first two highest scoring positions fails to produce an alteration in recognition, the first and third highest scoring positions may be altered, and the process repeated if necessary as indicated in Table 2 until the positions specifying recognition of the position-specific module are determined. In some cases it may be necessary to alter three or more positions to achieve alteration of the module recognized.

### DETAILED DESCRIPTION OF THE EMBODIMENTS

Present embodiments of the invention provide methods for rationally designing and making enzymes with novel recognition specificities, which have been selected or reliably predicted in advance. Catalogs based on correlations between position-specific amino acids in aligned binding proteins and position-specific modules in their recognition sequences in a substrate can be created. The catalog can be expanded by analyzing additional members of the set of binding proteins that recognize new combinations of modules in the recognition sequence or that contain an unexpected amino acid at a correlated position within the amino acid sequence. Using the catalog, large numbers of novel DNA binding proteins may be created based on various combinations of position-specific amino acid mutations.

Although the examples describe DNA binding proteins, the methods and compositions described herein are broadly applicable to any binding protein that recognizes a substrate that contains a characteristic position-specific sequence of modules recognized by the binding protein.

An overview of steps of an embodiment of the method is described in the flow diagram in FIG. 25A. A detailed description of multiple method steps of an analysis as executed for a set of DNA binding proteins is provided in FIG. 25B. Embodiments of the method may utilize one or more of the individual method steps described in each of boxes 1-8 in FIG. 25A and in each of boxes 1-41 in FIG. 25B and are not restricted to execution of the entire described set of method steps in FIG. 25A or 25B.

As described generally in the flow diagram in FIG. 25A and more particularly for a specific DNA binding protein in FIG. 25B, a polynucleotide may be generated that encodes a binding protein having an altered substrate specificity following steps that include: (a) identifying a set of closely related binding proteins having known amino acid sequences and preferably also having known module recognition specificity; (b) aligning the recognition sequences of the set of closely related binding proteins; (c) aligning the amino acid sequences of the set of closely related binding proteins; (d) identifying the position-specific amino acid residues that correlate with the position-specific module recognized by the members of the set of binding proteins; and (e) forming a novel binding protein that specifically recognizes a new, rationally chosen recognition sequence by changing the amino acid residue(s) of that protein identified by correlation as recognizing the module at a given position in the recognition sequence alignment. The identified amino acids can be changed to those amino acid residue(s) identified by correlation among members of the set that recognize a different module at the given position in the recognition sequence alignment. The exchange of amino acid residues may be accomplished by site-directed mutagenesis. By rationally altering the amino acid residues that confer specificity at the various positions within the recognition sequence, a very large number of proteins having specificity for novel recognition sequences may be created.

Embodiments of the method may be executed by a computer having been programmed to accomplish at least one of the steps outlined in either or both of FIGS. 25A and 25B. The predictions provided by computer analysis may be tested using high-throughput techniques that facilitate examination of large numbers of mutated proteins or by laboratory techniques that examine a small number of rationally designed proteins or examine single proteins.

The systems and methods described herein are amenable to complete automation using established devices for accomplishing the wet chemistry component can communicate with a computer for prior instructions as well as post-chemistry computation.

The computer would calculate steps 1-4, 6 and 7A in FIG. 25A. The device would perform the chemistry necessary for Boxes 5 and 7A in FIG. 25A sending data about binding of a mutated protein to a predetermined recognition sequence back to the computer, which could then process that data to confirm novel specificity, build iteratively the catalog and analyze novel binding proteins for hypothetical recognition sequences.

The instrument or device for conducting the wet chemistry steps might perform DNA synthesis and in vitro transcription and translation steps or alternatively directly synthesize a protein by programmed amino acid synthesis and then provide a high-throughput assay format known within the art (Kawahashi, et al. J Biochem 141:19-24 (2007)) for determining binding of multiple mutants to preselected recognition sequences such that the bound molecules emit a signal for detection, digitization and storage in a memory of a computer.

The method described herein is applicable to any protein that is capable of recognizing a specific sequence containing position-specific modules where the sequence or module may be represented for example by a nucleic acid, a monosaccharide, an amino acid or a chemical group. The methods described herein may be most broadly applied to any binding protein of which a DNA binding protein is a subset.

A “binding protein” as used herein may refer to a protein that binds to position-specific modules in a binding protein-specific recognition sequence. “Binding” means having an electrochemical attraction to or forming a covalent bond with the specific substrate sufficient to favor association in a disordered environment. Examples of binding proteins include those that bind biological macromolecules such as nucleic acid binding proteins for example, restriction endonucleases, homing endonucleases, and zinc finger proteins; RNA-binding proteins; carbohydrate-binding proteins; glycoprotein-binding proteins; glycolipid-binding proteins; lipid-binding proteins; and binding proteins that bind small molecules that contain a range of chemical groups or a single chemical group arranged in a specific predetermined order.

The term “module” is used generally to describe individual position-specific components in a specific recognition sequence, which forms a substrate for the binding protein.

A “substrate” as used herein refers to a molecule that has a number of modules having specific positions in a sequence, some or all of which are capable of having an electrochemical attraction to or forming a covalent bond with one or more specific amino acids in the binding protein. The number of different modules in a substrate may vary from 1 to as many as 20 modules or more, while a substrate may be composed of a few to millions or more modules.

“One or more specific amino acids” refers to a target of rational design where one or more optional changes of the target causes a change in the specificity of the protein to at least one module in the substrate. The one or more amino acids are likely to be a subset of the protein sequence required for binding the substrate.

“Prediction” as used herein refers to obtaining an improved approximation of accuracy of reproduction of alignment patterns.

“Correlation” may be used herein to mean an indication of the strength and direction of a linear relationship between two random variables. In general statistical usage, correlation or co-relation refers to the departure of two variables from independence. A statistically significant correlation may be calculated within the context of creating a catalog by using any one of a variety of tests such as a Chi square test, a mutual information analysis that for two random variables provides a quantity that measures the mutual dependence of the two (Gloor, et al. Biochemistry 44:7156-7165 (2005)) and a Pearson product-moment correlation coefficient (Spiegel, M. R. “Correlation Theory.” Ch. 14 in Theory and Problems of Probability and Statistics, 2nd ed. New York: McGraw-Hill, pp. 294-323, 1992).

“Set” is used herein as a related group of molecules of two or more members.

“Catalog” is a list of positionally defined amino acids that determine recognition of specific modules in a recognition sequence in a substrate.

“Recognition sequence” is a sequence of modules in a substrate, which is bound specifically by a binding protein.

“MmeI-like proteins” are proteins that belong to a set of amino acid sequences wherein each amino acid sequence in the set consists of part or all of a binding protein wherein the amino acid sequences (i) share an expectation value (E) of less than e-20 in a BLAST Search using MmeI as a query; and (ii) bind to specific DNA recognition sequences in a substrate, the DNA recognition sequences containing position-specific DNA bases.

Embodiments of the method may include one or more of the following steps:

1) Identify and collect a set or sets of closely related binding proteins for which both the sequence recognized by the protein and the amino acid sequence of the protein are known. Such a set of sequences may be identified in various ways. For example, a BLAST search of all sequences available in a database, such as Genbank, may be performed. Typically the query sequence is the amino acid sequence of a binding protein of interest, for example, in one such embodiment, a DNA binding protein exemplified here by MmeI restriction endonuclease may be used for the query. Alternatively, an amino acid sequence that is closely related to MmeI can be used to conduct a BLAST search. FIG. 16 shows the results of a Blast search using SpoDI which is closely related to MmeI which is used for a Blast search in FIG. 18. The Figures show that the results of the search are not identical. Performing multiple searches using different related proteins can result in the expansion of the set of aligned amino acid sequences.

The standard BLAST search blastp may be performed, although the parameters of the search may be varied by those skilled in the art. Because the method utilizes only closely related amino acid sequences, the standard blastp program search will identify sequences that can be usefully employed in the method. Alternative forms of the BLAST search may be performed, such as tblastn using the amino acid sequence of the starting query binding protein to search against translated nucleotide sequences in the database. This tblastn search is particularly useful for searching databases containing environmental DNA, and it is also useful to identify extended regions of similarity to the query binding protein when there are frameshifts or stop codons in the putative binding protein that cause the amino acid sequence reported in the database to be shortened relative to the full length query sequence. In another form of the BLAST search, the DNA sequence of the binding protein may be used to search either against protein sequences in the database (tblastp program), or against nucleotide sequences in the database (blastn program). The Expectation value from the BLAST search may be used to determine inclusion or exclusion of sequences from the set. Proteins that are only distantly related are unlikely to share enough sequence similarity to reliably align their sequences in order to observe residues and positions that correlate with module recognition. Requiring a relatively stringent BLAST E value threshold for inclusion in the chosen set of sequences ensures that distantly related sequences will be excluded.

The Expectation value chosen for inclusion in the set of related sequences is influenced by the length of the input sequence. For binding proteins having amino acid sequences longer than 200 amino acids, such as the majority of restriction endonucleases, an Expectation value of E<e-20 is employed. For shorter sequences, a larger E value is employed, such as E<e-10 for sequences between 100 and 200 amino acids in length.

The set of protein sequences employed may be further divided into subsets during the analysis in cases where this allows better alignment of the sequences within the subsets (fewer gaps and higher alignment scores), as this will reflect closer evolutionary and structural relationships between the members of the subsets, which will increase the likelihood that statistically significant correlations can be observed between amino acid residues and position-specific modules (e.g., DNA bases).

The sequences identified through the BLAST search may be sorted into those that have a known recognition sequence and those for which the sequence recognized is unknown. If there are sufficient protein sequences having known recognition sequences to produce statistically significant results, the analysis may be performed using these sequences. However, if there are not enough protein sequences for which the recognition sequence is known, then some of the identified putative binding proteins may have their recognition sequence determined biochemically (WO 2007/097778). This was the case for Example I, in which MmeI was used to identify homolog peptides in Genbank. The majority of the proteins identified in this search were uncharacterized as to their function, including their DNA recognition sequence specificity at the start of analysis. Therefore, a number of these peptides were characterized to determine their respective DNA recognition sequences, after which they were employed in the method described to create novel DNA binding proteins. For identified members of the binding protein set wherein the recognition sequence is not known, the recognition sequence may be determined biochemically. For example, a DNA recognition sequence for an uncharacterized member of the MmeI-like family of binding proteins may be determined by analyzing the location of DNA cutting and the size of the DNA fragments produced from various DNA substrates (Schildkraut Genet. Eng. 6:117-140 (1984)) or alternatively by analyzing the location of DNA modification in various DNA substrates.

An example of determining the DNA recognition sequence by characterizing the activity of the binding protein has been demonstrated for two related restriction endonucleases—CstMI and NmeAIII (see U.S. Pat. No. 7,186,538 and International Application No. PCT/US07/88522, respectively).

2) Align the recognition sequences of the binding proteins. The recognition sequences are preferably aligned to accurately reflect the nature of the interaction between the binding protein and the sequence recognized. To do this, the recognition sequence alignment is anchored about a common function.

For example, with respect to DNA binding proteins, the DNA recognition sequence will often consist of a different linear sequence of bases on each strand of the two strands in the DNA double helix. The exception to this is the case of DNA binding proteins that recognize symmetrical DNA sequences, in which the linear sequence of DNA bases recognized is the same from 5′ to 3′ in both DNA strands. It is important to choose the correct DNA strand to be aligned, since the two strands of the recognition sequence may have a different linear sequence of bases. The correct DNA strand is determined by the functional attribute(s) chosen to guide the alignment. For example, for restriction endonucleases, the functional attributes that enable accurate alignment of the DNA recognition sequences may consist of the methylation of a conserved adenine or cytosine base, and/or the direction of DNA cleavage downstream from the targeted specific DNA sequence recognized. In Example 1, the DNA recognition sequences were aligned using the strand containing the adenine base that is methylated, and which has the position of cleavage located 3′ to the recognition sequence on this strand. The alignment was fixed about this methylation target adenine. The linear sequence of bases in the second DNA strand is defined by the sequence of the strand employed in the alignment.

The position of methylation may be determined by incorporating a labeled methyl group such as radioactive tritium methyl group into various DNAs and mapping where the labeled methyl groups are located in the DNAs. Methylation can also be analyzed by protection against restriction endonucleases whose recognition sequences overlap the methylated base produced by the enzyme being characterized.

3) Align the amino acid sequences of the set of highly similar binding proteins. This may be done using any of a number of sequence alignment programs, such as ClustalW (http://www.ebi.ac.uk/clustalw/), PROMALS (http:prodata.swmed.edu/promals), MUSCLE (http://phylogenomics.berkeley.edu/cgi-bin/muscle/input_muscle.py), or T-Coffee (http://www.ebi.ac.uk/t-coffee/), or other similar programs. Generally the default alignment values of programs such as ClustalW or PROMALS algorithm may be used. The PROMALS algorithm is slower but provides improved alignment results. It should be understood that the skilled artisan may vary the parameters of the alignment programs to produce optimal alignment results, or the alignments may be refined manually by the skilled artisan. Since the method uses a set of closely related binding proteins, suitable alignments may be produced with the default settings of most widely used alignment programs. When one or more of the input binding protein sequences are less similar to the others, there may be a benefit to adjusting the alignment parameters or, if one or more sequences fails to align closely with the majority, or if it produces numerous gaps or otherwise degrades the alignment of the majority of sequences, such sequences may be excluded from the initial alignment in order to preserve the overall correctness of the amino acid sequence alignment produced.

4) Information contained in the recognition sequence alignment and the amino acid protein sequence alignment is combined to identify the amino acid positions, and the amino acids occurring at those positions, responsible for specific-sequence recognition.

The amino acid sequence alignment is interrogated to identify positions in which the amino acid residues present correlate with the module recognized by the binding proteins at a given position within the aligned DNA recognition sequences. A statistically significant, for example P<0.01, correlation indicates that specific module recognition is accomplished by the particular amino acid residue present at this position in the amino acid sequence of the binding protein. Recognition of a given base pair may require two or more amino acid residues located at different positions within the linear amino acid sequence of the protein. Such correlations may be identified using the computer program described in the examples, other similar programs. The skilled artisan may also identify such correlations by eye.

Embodiments of the method presented have the advantage of identifying amino acid positions that interact to recognize a given module even when the positions are widely separated in the primary amino acid sequence. Such widely separated positions are predicted to be spatially close in the three dimensional structure of the binding protein in order to recognize the given module.

Once correlations are observed, the respective amino acid residues are altered so as to recognize a different base pair at the position interrogated, and the altered proteins are tested for binding at the expected new recognition sequence. Successful identification of the amino acid residues conferring module specificity is confirmed by the altered binding protein, specifically binding the new, predicted recognition sequence (see for example FIGS. 1-9).

5) Rationally alter binding proteins such that they recognize novel recognition sequences. Once the amino acid residue positions and the individual amino acid residues that confer specificity for a given module at a given position within the recognition sequence are identified, novel binding proteins may be created by site-directed mutagenesis of the polynucleotide sequence encoding the identified amino acid residues. The amino acid residues at the positions conferring recognition specificity are specifically changed to those residues identified that specify recognition of the different, desired module in the recognition sequence. Such changes result in the creation of a binding protein that now predictably recognizes a new recognition sequence containing the position-specific module recognized by the altered residues. By employing combinatorial methods to change various combinations of the amino acid residues responsible for position-specific module recognition at different positions within the recognition sequence, large numbers of binding proteins that recognize novel recognition sequences may be synthesized (see FIG. 23).

### Uses of the Method

Embodiments of the method are powerful tools for using sequence data that is either new or already in sequence databases for: mining for enzymes with particular functions; analyzing functions of existing proteins; designing and creating novel enzymes with a desired specificity; and providing a rational means to increase the length of the specific recognition sequence for certain binding proteins, thereby conferring an increased specificity.

Rational design methodology can provide predictions of: the DNA recognition sequence of uncharacterized binding proteins in a set of proteins; a position-specific portion of the recognition sequence of uncharacterized binding protein sequences that match a set of characterized binding proteins with a defined relationship (E value); and/or rational design and creation of a binding protein with a desired recognition sequence.

New restriction endonucleases that recognize novel sequences provide greater opportunities and ability for genetic manipulation. Each new unique endonuclease enables scientists to precisely cleave DNA at new positions within the DNA molecule, with all the opportunities this offers. Such novel restriction endonucleases may enable detection of single nucleotide polymorphisms that previous restriction endonucleases could not differentiate. New recognition specificities enable new restriction fragment-linked polymorphism analysis as well as offer increased flexibility in cloning techniques that require specific DNA cutting and reassembly. The methyltransferase activity of the altered enzymes may also be used to introduce methyl or other chemical groups into DNA at the new specific recognition sequences. DNA may thus be specifically labeled at the various recognition sequences by the action of the novel enzymes. The introduction of methyl groups can also be used to block the action of restriction endonucleases where the site-modified overlaps the recognition sequence of the restriction endonuclease. Engineered methyl transferases may provide a useful resource for cloning naturally occurring restriction endonucleases for which no methylase is known to exist to protect the transformed host cells.

Methyl transferases with altered binding specificities may be used to introduce labels into DNA at specific sites. These labels may depend on the introduction of a methyl group or alternatively another chemical group.

### Prediction of Binding Specificity for Uncharacterized Proteins

There are often numerous uncharacterized homologs to a given set of characterized proteins in public databases, such as Genbank. The recognition sequences of the homologs are generally unknown. Without knowledge of the specific sequence recognized, these proteins cannot participate in the method described herein. However, once the position(s) within the set of amino acid sequences that determine recognition become known along with the module specificity determined by particular amino acid residues at these position(s), then the recognition specificity of these uncharacterized homologs can be predicted when their position-specific amino acid sequence matches residues conferring known module recognition at these positions.

### Identification in Naturally Occurring Protein Sequences of Likely Novel Position-Specific Module Recognition Sequences

Where the amino acid residues of the uncharacterized homologs do not match amino acid residues known to recognize certain modules, these homologs are identified as likely candidates to recognize a different module at these positions in the recognition sequence. Thus, the position-specific amino acid residues of those uncharacterized homolog proteins may be exchanged for the position-specific amino acid residues of a characterized binding protein, and the altered protein can then be characterized for binding specificity, with the expectation that it will likely bind to the recognition sequence with an altered module specificity at that particular position within the recognition sequence.

Position-specific amino acid residues known to confer specific recognition of a given module can be changed to alternative residues observed at these aligned positions in homologous protein sequences in the databases having an unknown recognition sequence. Such substitutions reflect the variety of naturally occurring binding proteins without requiring the foreknowledge of the specific recognition specificity of each such protein sequence. In this manner, recognition of modules not observed in the currently known recognition sequence may be obtained. An example of this embodiment is presented in Example 2, wherein the MmeI restriction endonuclease/methyltransferase is altered to generate an enzyme recognizing a novel DNA sequence. The amino acids that confer recognition of the DNA base pair at position 6 of the recognition sequence (E₈₀₆(S)R₈₀₈) were altered to those residues observed in several naturally occurring but uncharacterized sequences that align with the known position-specific residues, (G(N)G), which results in the creation of a restriction enzyme that recognizes a novel DNA binding sequence, 5′-TCCRAR-3′ (see FIGS. 6 and 23).

Generation of Novel Position-Specific Module Recognition Sequences by Random Mutagenesis of Identified Amino Acid Positions that Confer Position-Specific Module Specificity

The identification of positions within the binding protein sequence that confer DNA binding specificity allows for the alteration of the amino acid residues at these positions to all possible amino acid residues (see for example FIG. 23). This represents a rational, targeted mutation of those residues identified as conferring specificity. The proteins thus altered may then be tested biochemically to determine their recognition specificity to identify novel binding proteins. A major benefit of this approach is that it is easily tractable to change a few amino acid positions, such as the two positions conferring DNA base pair specificity at position 6 of MmeI restriction endonuclease (Example 1), whereas random mutagenesis of an entire protein sequence, or even a relatively small subset of that sequence, quickly becomes intractable due to the exponential number of mutations required. For example, randomly changing the two amino acid residue positions identified for MmeI position 6 would require 20×20, or 400 different sequences. In the case of zinc finger protein mutagenesis, randomly altering all seven amino acid positions believed to interact with DNA to form the recognition of the three base pair triplet recognized would require 20⁷, or 1.28×10⁹ different mutations (Durai, S. et al. NAR 33(18):5978-5990 (2005)). For combinations of zinc fingers to recognize longer DNA base pair sequences, such as 6 or 9 base pairs, the number of mutations required quickly becomes intractable (˜10¹⁸ for 6 base pairs, or ˜10²⁷ for 9 base pairs). Identifying those few amino acid positions that interact with the DNA to confer base specificity using the method presented herein allows the alteration of these identified residues to be performed, allowing identification of new DNA binding proteins that recognize novel DNA sequences.

### Generation of Binding Proteins Having Increased Module-Binding Specificity

When some members of the set of closely related binding-proteins specifically recognize more modules than other members of the set, the aligned recognition sequences and aligned amino acid sequences are examined to identify correlations between the position-specific amino acid sequence alignment and those recognition sequences that specify a particular module at a position where other recognition sequences do not recognize a specific module. In the example of the MmeI restriction endonuclease family, several of the members recognize a seven base pair sequence, while others recognize only six base pairs. For example, MmeI recognizes specific DNA bases in the four positions 5′ to the adenine that is methylated, as well as one base 3′ to that adenine, but does not recognize a specific base in the fifth position 5′ to the methylation target adenine, whereas SpoDI recognizes a specific DNA base, “G”, in the fifth position 5′ to the methylation target adenine in addition to recognizing specific bases in the four positions immediately 5′ to the methylation target adenine and one base 3′ to that adenine. The amino acid position(s) and position-specific amino acid residue(s) that confer specificity at this extended position are identified by the method of correlation described, wherein the correlation will consist of significant identities among those sequences that recognize a given DNA base at the extended position, while those sequences that do not specify any DNA base at the extended position will not exhibit such correlations. Using the method described herein, once the amino acid position(s) and residue(s) responsible for the specific recognition of the additional extra DNA base(s) are identified, the amino acid sequence responsible for this extra base recognition may be introduced by site-directed mutagenesis into the genes of the related DNA binding proteins recognizing a shorter recognition sequence to extend their specificity to include the additional base pair(s).

All references cited above and below, as well as U.S. provisional application No. 60/936,504 filed Jun. 20, 2007, are herein incorporated by reference.

### EXAMPLES

### Example 1

### Rational Generation of Novel Functional Type IIG Restriction Endonucleases that Specifically Recognize Novel DNA Sequences from MmeI, NmeAIII, SdeAI and Related Type IIG Restriction Endonucleases

MmeI is a DNA binding protein that specifically binds to the double-stranded DNA sequence 5′-TCCRAC-3′/5-GTYGGA-3′. MmeI functions to methylate the adenine base in the DNA strand 5′-TCCRAC-3′. MmeI also functions as an endonuclease, cleaving the double-stranded DNA 20 nucleotides 3′ to the TCCRAC strand and 18 nucleotides 5′ to the GTYGGA strand to leave a two base 3′ extension (1,2).

A set of polypeptides having members with a high degree of similarity to the Type IIG restriction endonuclease MmeI was identified through performing a BLAST search of the Genbank non-redundant database employing the blastp program (Altschul et al. J. Mol. Biol. 215:403-410 (1990); Altschul et al. Nucleic Acids Res. 25:3389-3402 (1997); and Madden et al. Methods Enzymol. 266:131-141 (1996)) (FIG. 18 and #1 in FIG. 25B-1). The MmeI amino acid sequence (U.S. Pat. No. 7,115,407) was used as query and a cut-off value for inclusion in the dataset of an Expectation score, E, of E<e-20 was employed. The default parameters of the NCBI web based blastp program were utilized (http://www.ncbi.nlm.nih.gov/BLAST/). A number of polypeptide sequences were identified as highly similar to MmeI; however, none of these sequences was characterized as to function, particularly regarding the specific DNA sequence recognized by the given polypeptide. Therefore, a number of these hypothetical sequences were cloned and expressed. The expressed proteins were tested for endonuclease activity, and the specific DNA sequence at which they bound DNA was characterized (U.S. Pat. No. 7,186,538). Among the set of sequences identified through the BLAST search as highly similar to MmeI, the specific DNA recognition sequence of the following active Type II endonucleases were identified. These enzymes also possess DNA methyltransferase activity.

CstMI, from Genbank Accession number GI:32479387, recognizes the DNA sequence 5′-AAGGAG-3′ and cuts 20 nucleotides 3′ to this sequence on this strand, and 18 nucleotides 5′ to the complement on the opposite DNA strand, to give a 2 base, 3′ extension: AAGGAGN20/N18(7).

NmeAIII, from Genbank accession number NC_{—}003116, peptide accession GI:15794682, was made active by correcting a stop codon within the reading frame identified as highly significantly similar to MmeI. NmeAIII was found to recognize 5′-GCCGAG-3′ and cut downstream: GCCGAGN21/N19 (international application no. PCT/US07/88522).

SdeAI, (formerly known as TdeAI) from Genbank accession number: NC_{—}007575.1, peptide accession YP_{—}392994.1, was cloned, expressed and characterized. SdeAI recognizes the DNA sequence 5′-CAGRAG-3′ and cuts downstream: CAGRAGN21/N19.

EsaSSI, from Genbank accession number AACY01071935.1, is an environmental DNA sequence from the Sargasso Sea, which meant that there was no available template DNA from which to amplify and clone the gene. Therefore, the gene encoding EsaSSI was made synthetically, and the amino acid codons for the peptide sequence were optimized to commonly used E. coli codons. The synthesized gene was assembled and cloned into E. coli, expressed and the enzyme activity characterized. EsaSSI was found to recognize the DNA sequence 5′-GACCAC-3′.

SpoDI, from Genbank accession number NC_{—}003911.11, peptide accession YP_{—}167160, was cloned, expressed and characterized to recognize the DNA sequence 5′-GCGGMG-3 and cut downstream GCGGAAGN20/N18.

DraRI, from Genbank accession number NC_{—}001264.1, peptide accession NP_{—}285443, was cloned; a false stop error in the gene was corrected by changing a TAA stop codon at position 2521 (amino acid position 841) to a GAA codon. The gene was expressed and the protein product characterized. DraRI was found to recognize the DNA sequence 5′-CAAGNAC-3′ and to cut downstream CAAGNACN20/N18.

ApyPI, from Genbank accession locus NC_{—}005206.1, protein accession NP_{—}940747, was cloned. A frameshift near the C-terminus of the protein was corrected using similarity to the CstMI protein to guide the correction position. The active, full-length protein and the corrected DNA sequence encoding this polypeptide were reported. The corrected ApyPI enzyme was expressed and characterized to recognize 5′-ATCGAC-3′ and to cut downstream ATCGACN20/N18.

PspPRI, from Genbank accession locus YP_{—}001274371, peptide accession NC_{—}009516.1, was cloned, expressed and characterized to recognize 5′-CCYCAG-3′ and to cut downstream CCYCAGN21/N19 or CCYCAGN20/N18.

NhaXI, from Genbank accession locus CP000319.1, peptide accession YP_{—}579008, was cloned, expressed and characterized to recognize 5′-CAAGRAG-3′ and to cut downstream CAAGRAGN20/N18.

CdpI, from Genbank accession locus NC_{—}002935.2, peptide accession: NP_{—}940094, was cloned, expressed and characterized to recognize 5′-GCGGAG-3′ and to cut downstream GCGGAGN20/N18.

RpaB5I, from Genbank accession locus NC_{—}007958.1, peptide accession YP_{—}570364, was cloned, expressed and characterized to recognize the DNA sequence 5′-CGRGGAC-3′ and cut downstream CGRGGACN20/N18.

NlaCI, from Neisseria lactamica ST640, was cloned, expressed and characterized to recognize 5′-CATCAC-3′, and to cut downstream CATCACN19/N17 or CATCACN20/N18.

DrdIV, from Deinococcus radiodurans NEB479, was cloned, expressed and characterized to recognize 5′-GCGGAG-3′ and to cut downstream GCGGAGN20/N18.

PspOMII, from Pseudomonas species OM2164, was cloned, expressed and characterized to recognize 5′-GCGGAG-3′ and to cut downstream GCGGAGN20/N18.

MaqI, from Genbank accession locus NC_{—}008738.2, peptide accession: YP_{—}956924, was cloned, expressed and characterized to recognize 5′-CRTTGAC-3′ and to cut downstream CRTTGACN20/N18.

PlaDI, from Genbank accession locus NC 009719.1, peptide accession: YP_{—}001413872, was cloned, expressed and characterized to recognize 5′-CATCAG-3′ and to cut downstream CATCAGN20/N18.

AquIII, from Genbank accession locus NC_{—}010475, peptide accession: YP_{—}001735369, was cloned, expressed and characterized to recognize 5′-GAGGAG-3′ and to cut downstream GAGGAGN20/N18.

AquIV, from Genbank accession locus NC_{—}010475, peptide accession: YP_{—}001735547, was cloned, expressed and characterized to recognize 5′-GRGGAAG-3′ and to cut downstream GRGGAAGN20/N18.

The DNA recognition sequences of MmeI and these newly characterized homolog enzymes were aligned. The alignment was made using the DNA strand that contains the adenine base, that is, modified by the DNA methyltransferase activity of these enzymes, and that is also the strand that is cleaved 3′ to the DNA recognition sequence. The DNA sequences were aligned so that the adenine base that is methylated is aligned for each enzyme. The DNA recognition sequence alignment is given in FIGS. 10 and 15 and #7 in FIG. 25B.

A multiple sequence alignment was constructed from the primary amino acid sequences of the highly similar restriction endonuclease polypeptide sequences having the known DNA recognition sequences described in FIG. 10. The alignment program ClustalW was used: http://www.ebi.ac.uk/clustalw/. The default settings were employed in the algorithm, except that the alignment was returned with the sequences in the input order, rather than the alignment score order. A portion of the multiple sequence alignment obtained is presented in FIG. 13 and #8 in FIG. 25B). A multiple sequence alignment for the entire amino acid sequences of the enzymes formed using the more rigorous alignment program PROMALS, http://prodata.swmed.edu/promals/promals.php, is shown in FIG. 20.

The polypeptide sequences were grouped according to the function of the DNA base recognized in the position 3′ to the methylation target adenine. The enzymes recognizing cytosine, “C”, are MmeI, EsaSS217I, ApyPI, NlaCI, DrdIV, RpaB5I, DraRI and MaqI. The enzymes recognizing guanine, “G”, at this position, are NhaXI, NmeAIII, CdpI, AquIII, CstMI, SdeAI, PspPRI, PlaDI, SpoDI and AquIV. PspOMII recognizes “R” at this position. The alignment was interrogated for amino acid residues at a given position in the alignment that were the same within the C and within the G group but which differed between the groups. For a small group of sequences such as this, the alignment can be examined manually, or interrogated by a computer program that can identify when there is a statistically significant correlation between the position-specific amino acid residues and the DNA base recognition. An example of such an algorithm is presented in FIG. 21. Upon examination of the alignment, one position was observed in which there was a 100% correlation between the amino acid residue present at this position and the DNA base recognized at this position within the DNA recognition sequence alignment. At this position, the cytosine is recognized by a group of amino acid sequences that has an Arginine residue, “R”, while the guanine recognizing group has an Aspartate residue, “D.” Both of these residues are charged and can readily form hydrogen bonds with DNA bases. The position of this residue in the MmeI sequence is R808, while in NmeAIII the residue is D818.

The candidate amino acid residue for recognizing cytosine, R808 in MmeI, and the equivalent position residue for recognizing guanine, D818 in NmeAIII, were changed to the amino acid residue expected to confer recognition of the other DNA base (R808 to D for MmeI and D818 to R for NmeAIII) by site-directed mutagenesis. For each enzyme, two oligonucleotide primers were synthesized for use according to the Phusion™ site-directed mutagenesis kit procedure (New England Biolabs, Ipswich, Mass.). For MmeI, the primers were: forward: 5′-pGATTATAGATATTCTGCCAGCCTGGTT-3′ (SEQ ID NO:27), where p is a phosphate, and reverse: 5′-pACTTTCTAACCTTCCTCCTACATTTCTC-3′ (SEQ ID NO:28). The first three nucleotides of the forward primer changed the amino acid codon for the arginine, “R808” of MmeI to a codon, “GAT” coding for aspartic acid, “D”.

The oligonucleotide primers to change NmeAIII were: forward: 5′-pCGCTATCGCTACTCTAATACCGTCGT-3′ (SEQ ID NO:29) and reverse: 5′-p GCTTTTCAGACGACCTGCAAC-3′ (SEQ ID NO:30). The first three nucleotides of the forward primer changed the coding of this position, D818, in NmeAIII from “D” to “R”. Mutagenesis was performed according to the manufacturer's directions and polynucleotides expressing the desired altered amino acid residue polypeptides were obtained. The altered MmeI polynucleotide, R808D, and the altered NmeAIII polynucleotide, D818R, were cloned into E. coli and expressed, but the polypeptides did not exhibit any restriction endonuclease activity. From this we concluded that they do not specifically bind the desired new recognition sequence, nor do they bind their original DNA recognition sequence, nor a different, unpredicted sequence. However, this position is likely to be involved in DNA recognition or some critical function or fold, since the altered proteins have lost the function of specific DNA binding.

Because it has been observed in other DNA binding proteins that specific base pairs are often recognized by two amino acid residues working cooperatively, the sequences were further examined for a second residue that would correlate with the recognition of the G or C base at the position immediately 3′ to the methylation target adenine. It was observed that the amino acid residue two positions toward the amino terminus of the polypeptides from the R or D position correlated, albeit with some variability, with the G or C base recognition. For those sequences recognizing the C base, this residue was most commonly a glutamic acid, “E”, while for those recognizing a G base, this residue was most often a lysine, “K”. This position thus has a charge opposite that of the “R” or “D” position identified as correlating 100% with the DNA base recognized, i.e., for the positive “R” residue correlating with the C base there is a negative charge “E” at this position, while for the negative “D” residue correlating with the G base there is a positive charged “K”. The two most diverged sequences, SpoDI and DraRI, both had different residues than the other members of their group at this position, with DraRI having a threonine residue, “T” rather than the “E”, while SpoDI has an insertion of two additional residues, glycine-valine, “GV”, immediately preceding the glycine “G” residue at this position. PspOMII had a “D” at this position, which forms a unique combination with the “D” residue at the 1:1 correlating position, which is consistent with the unique base recognition for PspOMII, “R”. Thus while the residues at this position (MmeI E806) were not the same within each base recognition grouping, they exhibited significant correlation with the DNA base recognized, and there was no example of the same residue present in more than one base recognition group. The amino acid residues at this second position identified (MmeI E806) were then altered in conjunction with that of the first position identified (MmeI R808) in order to change the DNA recognition at the base position following the methylation target adenine from C to G for MmeI, and from G to C for NmeAIII.

The correlated amino acid residues E806 and R808 in MmeI, and the equivalent position K816 and D818 in NmeAIII, were changed to the amino acid residue of the group recognizing the differing base by site-directed mutagenesis to generate the MmeI double mutant E806K, R808D, and the NmeAIII double mutant K816E and D818R. For each enzyme, two oligonucleotide primers were synthesized and used in the Phusion™ site-directed mutagenesis kit procedure. The MmeI primers were: forward: 5′-pGATTATAGATATTCTGCCAGCCTGGTT-3′ (SEQ ID NO:27), where p is a phosphate, and reverse: 5′-p ACTTTTTAACCTTCCTGCTACAGTTCTCATCCAGCAGTTGTGCA-3′ (SEQ ID NO:31). The primers to change NmeAIII were: forward: 5′-pCGCTATCGCTACTCTAATACCGTCGT-3′ (SEQ ID NO:29) and reverse: 5′-p GCTTTCCAGACGACCTCCAACGTTACGCATAAAGGCGTTGTG-3′ (SEQ ID NO:32).

Mutagenesis was performed according to the manufacturer's directions. The altered polynucleotides encoding the desired altered polypeptide sequences in their respective expression vectors were transformed into E. coli host cells. Two individual transformants of the altered MmeI and the altered NmeAIII were each inoculated into 30 ml of LB containing 100 micrograms/ml ampicillin and grown to mid-log phase, then IPTG was added to 0.4 mM and the cells were grown for two hours to induce expression of the altered protein. The cells were harvested by centrifugation, resuspended in 1.5 ml of sonication buffer SB (20 mM Tris, pH7.5, 1 mM DTT, 0.1 mM EDTA) and lysed by sonication. The extract was clarified by centrifugation. To test for endonuclease activity, serial dilutions of the extract were performed in NEBuffer 4, using pBC4 DNA (New England Biolabs, Inc., Ipswich, Mass.) linearized with NdeI as the DNA substrate. Discrete banding was observed for the altered MmeI, E806K and R808D, and the altered NmeAIII, K816E and D818R, indicating that the altered polynucleotide sequences encoded active endonucleases (FIGS. 1 and 2, and #14 and #17 in FIG. 25B).

### Characterization of the Altered MmeI DNA Recognition Sequence

The crude extract for the altered MmeI was purified over a 1 ml Heparin HiTrap column (GE Healthcare, Piscataway, N.J.). The 1.5 ml crude extract was applied to the column, which had been previously equilibrated in buffer A (20 mM Tris pH7.5, 1 mM DTT, 0.1 mM EDTA) containing 50 mM NaCl. The column was washed with 5 column volumes of buffer A containing 50 mM NaCl, then a 30 ml linear gradient in buffer A from 0.05M NaCl to 1M NaCl was applied and 1 ml fractions were collected. The altered MmeI was eluted at approximately 0.48M NaCl. It was expected that the rationally changed MmeI enzyme would recognize 5′-TCCRAG-3′. To determine the DNA recognition sequence for the altered polypeptide, the positions of cleavage for the purified enzyme were mapped on pBR322 DNA (FIG. 1 and #17 in FIG. 25B). The DNA was cut with the purified MmeI mutant, purified, and then were cut with an enzyme that cleaves once at a known position. The size of the unique fragments produced by the double digestion of the DNA showed the distance from the location of the known enzyme cutting position to the position of cutting by the MmeI mutant enzyme. The altered MmeI enzyme cutting positions on pBR322 were mapped to approximate positions 260, 310, 1340 and 2790. The sequence TCCRAG occurs in pBR322 at positions 276, 330, 1314 and 2772, which matches the observed cutting positions. The wild type MmeI recognition sequence, TCCRAC, occurs in pBR322 at positions 197, 283, 2662 and 2846, which did not match the observed cutting positions. The pattern of DNA fragments produced from endonuclease cleavage of phage lambda DNA, phage T3 DNA, pBC4 (Schildkraut Genet. Eng. 6:117-140 (1984)).) DNA and phage PhiX DNA was determined to match cleavage at the new recognition sequence TCCRAG (FIG. 1). These results indicate that the DNA base recognized by the altered MmeI at position six has been changed from C to G, as predicted by the rational, site-directed change of the amino acid residues at the positions identified as correlating with recognition of the DNA base at the 3′-most position in the recognition sequence alignment. The altered MmeI restriction endonuclease binds at the novel DNA sequence 5′-TCCRAG-3′ and cleaves the DNA 20 nucleotides 3′ to this sequence on this strand, and 18 nucleotides 5′ to the complementary sequence of the opposite strand 5′-CTYGGA-3′ to leave a two base, 3′ overhang. Application of the method resulted in the creation of a novel restriction enodnuclease.

### Characterization of the Altered NmeAIII DNA Recognition Sequence

The crude extract for the altered NmeAIII was used directly to map the cutting positions of this endonuclease in various DNAs. It was predicted that the rationally altered NmeAIII would recognize 5′-GCCGAC-3′. To determine the DNA recognition sequence for the altered polypeptide, the positions of cleavage for the altered enzyme were mapped on pBR322, PhiX174 and pBC4 DNAs (FIG. 2 and #17 in FIG. 19B). DNA was digested with the altered NmeAIII enzyme, purified on a spin column. The size of the unique fragments produced by the double digestion of the DNA indicated the distance from the location of the known enzyme cutting position to the position of cutting by the NmeAIII mutant enzyme.

The altered NmeAIII enzyme cut pBR322 at positions approximately 450 and 950. The sequence GCCGAC occurs in pBR322 at positions 446 and 941, which matches the observed cutting positions. The wild type NmeAIII recognition sequence, GCCGAG, occurs in pBR322 at positions 120, 1172 and 3489, which differed from altered NmeAIII recognition sequence. Similarly for phiX174 DNA, altered NmeAIII-cut positions in PhiX174 were mapped to approximately 2300, 2675, 3435, 4740 and 5335. The expected NmeAIII-altered recognition sequence, GCCGAC, occurs at positions 2251, 2641, 3474, 4710 and 5298, which matched the observed position of cutting. The wild type NmeAIII recognition sequence occurred in PhiX174 at positions 1022, 3426 and 4680, which differed from the recognition sequence of the altered NmeAIII. Similar results were obtained for pBC4 DNA mapping. These results indicated that the recognition sequence of NmeAIII was altered from G to C at the final base position as predicted by our rational, site-directed change of the amino acid residues found to correlate to the DNA base recognized at this position. These results are examples of how a directed change of the recognition sequence of a restriction endonuclease can be achieved where the amino acid residues confer specificity for a DNA base altered in a rational way to generate a predictable new DNA recognition specificity. The recognition specificity of SdeAI has also been changed through application of the same method from 5′-CAGRAG-3′ to 5′-CAGRAC-3′ (FIG. 9).

### Example 2

### Position-Specific Mutagenesis to Create a Novel DNA Recognition Sequence

Identification of the two positions within the amino acid sequence alignment of the set of proteins that determine recognition of the first base at the 3′ end in the aligned recognition sequences enabled the creation of novel restriction endonucleases using two approaches. In the first approach, the amino acid residues for all members of the set, including those for which the recognition sequence has not yet been determined, were aligned. The alignment was examined at the identified positions responsible for recognition to see if there were any naturally occurring variations that did not match the amino acids known to specify recognition of a given base (FIG. 12 and #32 in FIG. 25B). In the case of the characterized enzymes in Example 1, the amino acids at the alignment positions determining recognition at the position of the first base at the 3′ end of the DNA recognition sequence for nucleotide “C” were ExR and TxR. Those amino acids determining recognition of a G were KxD and GxD. The aligned members of the set were examined and several amino acid combinations that were not one of these C or G determining combinations were observed. Two of these amino acid residue combinations, GxS observed in Genbank accession number gi|28373198, and GxG, observed in Genbank accession number gi|87198286, were introduced into the MmeI polypeptide by site-directed mutagenesis, using the same procedure as in Example 1.

To introduce coding for the GxS amino acid combination into the polynucleotide encoding the MmeI protein, two oligonucleotide primers were synthesized and used in the Phusion™ site-directed mutagenesis kit procedure. The primers utilized were forward: 5′-pCGATATTCTGCCAGCCTGGTTTACAACAC-3′ (SEQ ID NO:165), where p is a phosphate, and reverse: 5′-pGTAACTAGTACCTAACCTTCCTCCTACATTTCTCATCCAGCA-3′ (SEQ ID NO:166). The reverse primer introduced the directed mutations into the MmeI gene. Mutagenesis was performed according to the manufacturer's directions. The same procedure was followed to introduce the GxG combination of position-specific amino acid residues into MmeI, using as primers: forward: 5′-pCGATATTCTGCCAGCCTGGTTTACAACAC-3′ (SEQ ID NO: 167), where p is a phosphate, and reverse: 5′-pGTAACCGTTACCTAACCTTCCTCCTACATTTCTCATCCAGCA-3′ (SEQ ID NO:168). The altered polynucleotides in the expression vector pRRS, encoding the desired altered polypeptide sequences, were transformed into E. coli host cells. One individual transformant of each altered MmeI were each inoculated into 30 ml of LB containing 100 micrograms/ml ampicillin and grown to mid-log phase, then IPTG was added to 0.4 mM and the cells were grown for two hours to induce expression of the altered protein. The cells were harvested by centrifugation, resuspended in 1.5 ml of sonication buffer SB (20 mM Tris, pH7.5, 1 mM DTT, 0.1 mM EDTA) and lysed by sonication. The extract was clarified by centrifugation. To test for endonuclease activity, the crude extract was used to cut PhiX174 DNA in NEBuffer 4 (New England Biolabs, Inc., Ipswich, Mass.) supplemented with SAM (80 micromolar). The cleaved DNA was purified over a Zymo Research “DNA Clean and Concentrate” spin column according to the manufacturer's instructions (Zymo Research, Orange, Calif.). The purified cut DNA was then used for mapping by cutting with four different known endonucleases. Discrete banding was observed for both the altered MmeI, E806G plus R808S, and the E806G plus R808G constructs, indicating that the altered polynucleotide sequences encoded active endonucleases.

The altered MmeI E806G plus R808G enzyme cut pUC19 at positions approximately 1135 and 1335 (FIG. 6A and #36 in FIG. 25B). The sequence TCCRAR occurs in pUC19 at positions 1105 (TCCRAG) and 1352 (TCCRAA), which matches the observed cutting positions. The wild type MmeI recognition sequence, TCCRAC, occurs in pUC19 at positions 996 and 1180, which did not match the positions observed for the altered enzyme. For pBR322 and phiX174 DNA, similar results were obtained (FIG. 6B). The altered enzyme cut positions in PhiX174 were mapped to approximately 25, 500, 3600, 3835 and 4135. The TCCRAR sequence occurs near these positions at 41, 471, 518, 3588, 3606, 3857 and 4143, which matches the observed position of cutting. The TCCRAR sequence also occurs at additional positions, 1510, 1671, 2998, 3959 and 3970. While cutting was not observed at these positions, the amount of enzyme available for cutting was limited and thus the digestion of the DNA was incomplete. The sites mapped were consistent with the altered enzyme cutting at TCCRAR, and were not consistent with cutting at the wild type unaltered specificity, TCCRAC, indicating the altered enzyme cleaves at a new specificity, namely TCCRAR.

### Example 3

### Creation of Enzymes that Recognize Novel DNA Recognition Sequences

Further enzymes that specifically recognize new DNA sequences were formed and characterized using the methods exemplified in Example 1 and 2 above. The oligonucleotide primers used for site-directed mutagenesis are shown in Table 1.

One such enzyme recognizing 5′-TCCGAC-3′ was formed by site-directed mutagenesis of MmeI, changing alanine 774 to leucine, using primers SEQ ID NO:151 and SEQ ID NO:152. The recognition specificity of this altered enzyme is demonstrated in FIG. 3.

Another such enzyme recognizing 5′-TCCCAC-3′ was formed by site-directed mutagenesis of MmeI, changing alanine 774 to lysine using primers SEQ ID NO:153 and SEQ ID NO:154, followed by altering arginine 810 to serine using primers SEQ ID NO: 155 and SEQ ID NO:156. The recognition specificity of this altered enzyme is demonstrated in FIG. 4.

Another new enzyme recognizing 5′-TCGRAC-3′ was formed by site-directed mutagenesis of MmeI, changing glutamate 751 to arginine and asparagine 773 to aspartate, using primers SEQ ID NO:157 and SEQ ID NO:158. The recognition specificity of this altered enzyme is demonstrated in FIG. 5.

Another new enzyme recognizing 5′-TCCRAB-3′ was formed by site-directed mutagenesis of MmeI, changing glutamate 806 to glycine and arginine 808 to threonine, using primers SEQ ID NO:159 and SEQ ID NO:160. The recognition specificity of this altered enzyme is demonstrated in FIG. 7.

Another new enzyme recognizing 5′-TCCRAN-3′ was formed by site-directed mutagenesis of MmeI, changing glutamate 806 to trytophan and arginine 808 to alanine, using primers SEQ ID NO:161 and SEQ ID NO:162. The recognition specificity of this altered enzyme is demonstrated in FIG. 8.

Another new enzyme recognizing 5′-CAGRAC-3′ was formed by site-directed mutagenesis of SdeAI, changing lysine 791 to glutamate and aspartate 793 to arginine, using primers SEQ ID NO:163 and SEQ ID:164 The recognition specificity of this altered enzyme is demonstrated in FIG. 9.

TABLE 1
List of oligonucleotide primers

| | | | |
|---|---|---|---|
| Mme4GI | A774L | CTGACGTATCATATTCCTAGTGCTGAAC | FIG. 3 |
| | | CT | |
| | | (SEQ ID NO:151) | |
| | | and | |
| | A774L | GTTACTTGAAATGACATTTCTATCAACAA | |
| | | AAC | |
| | | (SEQ ID NO:152)) | |
| Mme4CI | A774K | AAGACGTATCATATTCCTAGTGCTGAAC | FIG. 4 |
| | | CT | |
| | | (SEQ ID NO:153) | |
| | | and | |
| | A774K | GTTACTTGAAATGACATTTCTATCAACAA | |
| | | AAC | |
| | | (SEQ ID NO:154) | |
| | R810S | AGCTATTCTGCCAGCCTGGTTTACA | |
| | | (SEQ ID NO:155) | |
| | | and | |
| | R810S | GTAACGACTTTCTAACCTTCCTCCTACA | |
| | | (SEQ ID NO:156) | |
| Mme3GI | E751R | CAATTGGAATAAATTGTCTGTTTTCAGAT | FIG. 5 |
| | | GATGTGCGAGGTATCAACAGATAGTCCGT | |
| | | ATCCG | |
| | | (SEQ ID NO:157) | |
| | | and | |
| | N773D | GTTTTGTTGATAGAAATGTCATTTCAAGT | |
| | | GACGCAACGTATCATATTCCTAGTGCTGA | |
| | | AC | |
| | | (SEQ ID NO:158) | |
| Mme6BI | E806G | GCTGCCTAACCTTCCTCCTACATTTCTCA | FIG. 7 |
| | | TCCA | |
| | | (SEQ ID NO:159) | |
| | | and | |
| | R808T | ACCTATAGATATTCTGCCAGCCTGGTTTA | |
| | | CA | |
| | | (SEQ ID NO:160) | |
| Mme6NI | R808A | GTGCCTATAGATATTCTGCCAGCCTGGTT | FIG. 8 |
| | | TACA | |
| | | (SEQ ID NO:161) | |
| | | and | |
| | E806W | TCCATAACCTTCCTCCTACATTTCTCATC | |
| | | CA | |
| | | (SEQ ID NO:162) | |
| SdeA6CI | D793R | CGTTATTCAAATGAAATTGTTTATAACAA | FIG. 9 |
| | | CTTCCCT | |
| | | (SEQ ID NO:163) | |
| | | and | |
| | K791E | GTAACGACTTTCTAATCTTCCAGCAACAT | |
| | | ACCGCA | |
| | | (SEQ ID NO:164) | |

In summary, Examples 1, 2 and 3 demonstrate alteration of a DNA binding protein to recognize a novel DNA sequence through identifying the positions in the DNA binding protein that determine position-specific DNA base recognition and alteration of those positions to differing amino acid residues observed in uncharacterized naturally occurring sequences.

### Example 4

### Prediction of DNA Recognition Specificity for Uncharacterized DNA Binding Proteins

Once the position(s) within an amino acid alignment and the specific amino acid residues at those position(s) that confer position-specific DNA base recognition were identified, the DNA recognition specificity of uncharacterized polypeptides homologs could be accurately predicted. We have shown that the amino acids ExR corresponding to positions E806-(S)-R808 in MmeI specify recognition of a “C” in the DNA recognition sequence position immediately 3′ to the methylation target adenine in the family of homolog sequences related to MmeI. Any homolog found in a database, such as Genbank, that has the same amino acid residues, ExR at this position in the amino acid sequence alignment within the MmeI family of polypeptides is predicted with a high degree of certainty to recognize a “C” at this position. Similarly, the presence of the residues “KxD” at this position predicted that the polypeptide would recognize a “G” at this position. Variations in correlation of amino acids with type and position of nucleotide in the recognition sequence could be factored into the prediction. For example, residues “TxR” (from DraRI) had a predicted recognition of “C”, while “GVGND” (from SpoDI) had a predicted recognition of “G.” This prediction scheme has provided accurate predictions of DNA bases that are recognized for all members of the set characterized to date, such as EsaSSI where the DNA recognition sequence was found experimentally to be 5′-GACCAC-3′, and in which C was correctly predicted at the 3′-most position (FIG. 10A).

### Example 5

### Assembly of the Methyltransferase Family

The gamma-class N6A DNA methyltransferases shown in FIG. 22 were assembled by collecting sequences of enzymes for which the specific DNA recognition sequence was known and that recognized six DNA bases from the list of gamma class adenine methyltransferases in the REBASE database. The collected amino acid sequences were aligned using the PROMALS algorithm (http://prodata.swmed.edu/promals/promals.php). The DNA recognition sequences were aligned, placing the adenine that is presumed to be the modified adenine at position 5 of the alignment. The position in the aligned amino acid sequences identified by the box is significantly correlated with the DNA base recognized at position 3 of the recognition sequence alignment (Chi square P value <0.001). This is an example of using the method described to identify recognition sequence determinants in a family of proteins other than the MmeI-like family.

What is claimed is:

1. A method, comprising:
(a) creating a set of binding proteins using an initial binding protein to query a database in a BLAST search, wherein each binding protein has a defined amino acid sequence, such that the set of amino acid sequences share an expectation value (E) of less than e-20 for sequences of more than 200 amino acids or less than e-10 for sequences of less than 200 amino acids in the BLAST search; each binding protein binding to a specific target recognition sequence in a substrate, the target recognition sequences containing position-specific modules;
(b) aligning the target recognition sequences recognized by the binding proteins in the set;
(c) aligning the amino acid sequences of the binding proteins of the set; and
(d) identifying correlations between the aligned position-specific modules in the recognition sequences and one or more position-specific amino acids in the aligned amino acid sequences of the binding proteins.

2. A method according to claim 1, wherein step (b) further comprises: aligning by means of a position dependent feature in the specific target recognition sequence.

3. A method according to claim 1, further comprising: expanding the set of binding proteins by using a member of the set of binding proteins to query the database in an additional BLAST search.

4. A method according to claim 1, further comprising: identifying, in a plurality of the binding proteins in the set, the position and type of an amino acid residue or amino acid residues that determine recognition of one or more position-specific modules in the recognition sequence.

5. A method according to claim 4, further comprising: the step of creating a catalog for recording the positions of the amino acids in the aligned amino acid sequences and the amino acid residues at those positions that determine recognition of the specific types of modules at specific positions in the aligned recognition sequences of the set of binding proteins.

6. A method according to claim 5, further comprising: the step of using the catalog to rationally modify the amino acid sequence of one or more of the aligned binding proteins to recognize an altered specific target recognition sequence.

7. A method according to claim 4, further comprising: mutating non-randomly one or more amino acids at correlated positions in a single binding protein to cause a predictable change in the specific target recognition sequence of the binding protein.

8. A method, according to claim 1, wherein a binding protein member of the set has a known amino acid sequence but an uncharacterized specific target recognition sequence, further comprising the steps of:
(a) identifying position-specific modules in the recognition sequence by:
(i) reviewing the alignment of the amino acid sequence of the binding protein member in the aligned set of binding proteins;
(ii) reading out amino acid residues at the positions recorded in the catalog; and
(iii) comparing the amino acid residues in the binding protein member to the amino acid residues recorded in the catalog; and
(b) determining the specific target recognition sequence of the binding protein member.

9. A method according to claim 1, wherein the position-specific modules consist of one or more nucleotides in a DNA substrate.

10. A method according to claim 1, wherein the set of binding proteins is a set of DNA binding proteins.

11. A method according to claim 9, wherein the set of DNA binding proteins is a set of MmeI-like proteins.

12. A method according to claim 10, further comprising: changing the DNA recognition sequence of an MmeI-like DNA binding protein by changing the amino acid residues at a predetermined position or positions in the amino acid sequence of MmeI or an equivalent aligned position in an MmeI-like protein of a DNA binding protein.

13. A method according to claim 12, wherein the predetermined positions in the amino acid sequence of MmeI are selected from 751+773, 806+808, 774+810, 774, 774+810+809 and 809.

14. A method according to claim 11, wherein changing the recognition sequence further comprises: changing nucleotides at one or more of positions 3, 4 and 6 of the DNA recognition sequence.

15. A method according to claim 1, further comprising: storing the amino acid sequences for the set of binding proteins in a database in a computer-readable memory and performing one or more of steps (a), (b), (c) or (d) by executing instructions stored in a computer.

16. A method according to any of claims 3, 4 and 6, further comprising: performing the steps by executing instructions stored in a computer.

17. A method for generating a binding protein that recognizes a rationally chosen recognition sequence, comprising:
substituting a first amino acid with a second amino acid using site-directed mutagenesis of a member protein of a set of proteins at an identified position or positions correlated with recognition of a chosen specified target module.

18. A method for automating one or more steps in the flow diagram in FIG. 25A, comprising: utilizing a computer having programmed instructions to achieve one or more functions described in boxes 1, 2, 3, 4, 6, and 7B; and further utilizing an instrument capable of performing reactions to achieve any of steps 5, 7A or 8.

19. A method for automating one or more steps in the flow diagram in FIG. 25B using a computer for executing instructions and optionally automating one or more steps comprising chemical reactions.

20. An MmeI-like enzyme having a mutation resulting in at least one altered amino acid residue at a predetermined position that has a specificity for a DNA recognition sequence that is different by at least one base compared with the DNA recognition sequence of the unaltered enzyme.

21. An enzyme according to claim 20, wherein the difference of at least one base consists of a deletion or addition of a base.

22. An enzyme according to claim 20, wherein the difference consists of an alternative recognized base at an identified position in the recognition sequence.

23. A system comprising: a memory for storing instructions and a computer for executing the instructions, which when executed:
create a set of binding proteins using an initial binding protein to query a database in a BLAST search, wherein each binding protein has a defined amino acid sequence, the amino acid sequences sharing an expectation value (E) of less than e-20 for sequences of more than 200 amino acids or less than e-10 for sequences of less than 200 amino acids; the binding proteins binding to specific target recognition sequences in a substrate, the target recognition sequences containing position-specific modules;

24. A system according to claim 23, further comprising instructions, which when executed:
align the specific target recognition sequences recognized by the binding proteins; and align the amino acid sequences of the binding proteins of the set.

25. A system according to claim 24, further comprising instructions, which when executed:
identify correlations between the aligned position-specific modules in the recognition sequences and one or more position-specific amino acids in the aligned amino acid sequences of the binding proteins.

26. A system according to claim 25, further comprising: a means for receiving data from a device for protein synthesis and protein binding analysis and containing instructions, which when executed
use the data to validate the correlations by confirming a prediction of binding to a predetermined recognition sequence by a mutated protein; and
organize the data into a catalog of validated amino acid or amino acids at identified positions that determine recognition for a position and type of module in the recognition sequence.

27. A system comprising: a memory for storing instructions and a computer for executing the instructions, which when executed:
(a) collect and align a sorted set of amino acid sequences of binding proteins in a first database, and collect and align a sorted set of recognition sequences for at least a subset of the binding proteins in a second database, wherein the first database is obtained from an automated search of a third database of amino acid or nucleotide sequences;
(b) identify correlations between amino acids at selected aligned positions in the set of amino acid sequences and modules at selected aligned positions of modules in the recognition sequences;
(c) from an instrument for protein synthesis and protein binding analysis receive data on the correlations for using the data to validate the correlations by confirming a prediction of binding to a predetermined recognition sequence by a mutated protein; and
(d) organize the data into a catalog of validated amino acid or amino acids at identified positions that determine recognition for a position and type of module in the recognition sequence.

28. A system comprising: a memory for storing instructions and a computer for executing the instructions, which when executed:
store positional information of an amino acid residue or amino acids residues in a first binding protein for targeted mutation to create a second binding protein having a predicted alteration of a module in a sequence position within a sequence of modules recognized by the protein.

29. A system according to claim 28, wherein the stored instructions comprise the instructions in FIG. 7A.

30. A method or composition, comprising: any of the features disclosed in the attached description.
